(19) United States
(12) Patent Application Publication
(10) Pub. No.: US 2023/0220100 A1
(43) Pub. Date: Jul. 13, 2023

(54) BBB-TARGETED GAA DELIVERED AS GENE THERAPY TREATS CNS AND MUSCLE IN POMPE DISEASE MODEL MICE

(71) Applicant: Regeneron Pharmaceuticals, Inc., Tarrytown, NY (US)

(72) Inventors: Katherine Cygnar, New York, NY (US); Maria Praggastis, Cortlandt Manor, NY (US)

(21) Appl. No.: 18/152,447

(22) Filed: Jan. 10, 2023

Related U.S. Application Data
(60) Provisional application No. 63/298,018, filed on Jan. 10, 2022.

(51) Int. Cl. C07K 16/28; C12N 9/26; C12N 15/86

(52) U.S. Cl. CPC C07K 16/2881; C12N 9/2408; C12Y 302/0102; C12N 15/86; C07K 2317/622; C07K 2319/00; C07K 2319/33; C12N 2750/14143; C12N 2750/14171; A61K 48/00

(57) ABSTRACT

Compositions and methods for delivering a therapeutic protein to the central nervous system (CNS), in order to treat diseases and disorders that impair the CNS, such as treating lysosomal storage diseases, are disclosed. Therapeutic proteins delivered via a therapeutically effective amount of a nucleotide composition encoding the therapeutic protein conjugated to a cell surface receptor-binding protein, e.g., anti-TfRCscfv:GAA, that crosses the blood brain barrier (BBB) are provided.

### CROSS-REFERENCE TO RELATED APPLICATIONS

This application claims the benefit under 35 USC § 119(e) of U.S. Provisional Application Ser. No. 63/298,018, filed Jan. 10, 2022, which application is hereby incorporated in its entirety by reference.

### REFERENCE TO A SEQUENCE LISTING SUBMITTED AS A TEXT FILE VIA EFS-WEBS

The Sequence Listing written in file 11146US01_ST26.xml is 41 kilobytes, was created on Jan. 10, 2023, and is hereby incorporated in its entirety by reference.

### FIELD

This application is generally directed to compositions and methods for delivering a therapeutic protein to the central nervous system (CNS), in order to treat diseases and disorders that impair the CNS, such as treating lysosomal storage diseases (LSDs). This application is directed to providing a therapeutically effective amount of a nucleotide composition encoding a therapeutic protein conjugated to one or more delivery domains that crosses the blood brain barrier (BBB).

### BACKGROUND

Drug delivery approaches have been developed to overcome the blood brain barrier (BBB), however many approaches, such as nanocarriers, have shortcomings. Carriers have exhibited instability in blood circulation and undesirable biodistribution profiles (Gelperina et al., 2005, Am J Respir Crit Care Med. 172(12):1487-90; which reference is incorporated herein in its entirety by reference). Targeting efficiencies also have been compromised depending on the trafficking mechanisms at the BBB and whether a CNS disease state has altered the integrity of the barrier. Proper selection of the targeting moiety or carrier must take into consideration any neuroinflammatory conditions that affect these trafficking mechanisms.

Delivery of therapeutic proteins via DNA expression in the liver or other tissues has provided a convenient approach eliminating the need for bolus injection of protein and therefore lessening immunogenicity concerns. A therapeutic protein conjugated to a receptor binding protein, especially a cell-specific receptor, can solve some problems related to targeting therapeutics to specific tissues. However, there is still a need to provide compositions and methods that efficiently provide therapeutics to the CNS.

### SUMMARY

Applicants have discovered that therapeutic proteins, especially replacement enzymes, can be effectively delivered into the central nervous system (CNS) when associated with a receptor binding protein, and provided that the circulating blood levels achieve consistent levels over time. Multidomain therapeutic proteins can be delivered to the liver via a gene therapy vector harboring the coding sequence of the therapeutic protein and binding protein complex.

In one aspect, the invention provides a method of delivering a therapeutic protein to the CNS of a subject, comprising administering to the subject a nucleotide composition encoding the therapeutic protein (tp) conjugated to a cell surface receptor (CSR)-binding protein (CSR-BP) (tpCSR-BP) via a liver-targeted delivery method sufficient to provide a therapeutically effective amount of the tpCSR-BP in the CNS.

In one embodiment the CSR-BP is an antibody or antigen-binding fragment thereof that binds specifically to the CSR. In another embodiment, the therapeutic protein is a lysosomal enzyme.

In one embodiment, the enzyme has hydrolase activity, such as a glycosylase, such as a glycosidase, such as an alpha-glucosidase or alpha-galactosidase A. In one embodiment, the cell surface receptor (CSR)-binding protein (CSR-BP) is an antigen-binding protein that binds to an internalization receptor. In one embodiment, the internalization receptor is a cell-surface molecule that is endocytosed and trafficked to the lysosome. In a specific embodiment, the internalization receptor is a CD63 molecule. In one embodiment, the internalization receptor is a TfR molecule. In a specific embodiment, the CSR-BP is an antibody, an antibody fragment, or a single-chain variable fragment (scFv), such as an scFv that binds CD63 or TfR.

### DRAWINGS

The patent or application file contains at least one drawing executed in color. Copies of this patent or patent application publication with color drawing(s) will be provided by the Office upon request and payment of the necessary fee.

FIG. 1A schematically represents multidomain therapeutic proteins. Panel A depicts a multidomain therapeutic protein comprising a bispecific antibody (ii) and a replacement enzyme (i). Panel B depicts an enzyme-Fc fusion polypeptide (i) associating with an internalizing effector-specific half-body (ii) to form the multidomain therapeutic protein. Panel C depicts a replacement enzyme (hexagon) covalently linked to the C-terminus of the heavy chain of an anti-internalizing effector antibody. Panel D depicts a replacement enzyme (hexagon) covalently linked to the N-terminus of the heavy chain of an anti-internalizing effector antibody. Panel E depicts a replacement enzyme (hexagon) covalently linked to the C-terminus of the light chain of an anti-internalizing effector antibody. Panel F depicts a replacement enzyme (hexagon) covalently linked to the N-terminus of the light chain of an anti-internalizing effector antibody. Panel G depicts a replacement enzyme (hexagon) covalently linked to the C-terminus of a single-chain variable fragment (scFv) containing a VH region (shaded bar) and a VL region (open bar). Panel H depicts a replacement enzyme (hexagon) covalently linked to two scFv domains, the first scFv (i) which serves as a first delivery domain, and the second scFv (ii) which serves as a second delivery domain. Additional multidomain therapeutic proteins not depicted in FIG. 1A include, but are not limited to, multidomain therapeutic proteins comprising two or more delivery domains and at least one enzyme domain. As nonlimiting examples, the antibodies, half-bodies and scFv domains depicted in panels A-H of this figure may represent any type of delivery domain, and additional delivery domains or replacement enzymes can be also associated to make a multidomain therapeutic protein. Nonlimiting examples of multidomain therapeutic proteins comprising two or more delivery domains are further depicted in FIGS. 1C, 1D, and 1F, which include a replacement enzyme (depicted as, but not limited to, GAA) covalently linked to a first internalizing effector-specific half-body, which associates with a second internalizing effector-specific scFv-Fc fusion, which may or may not also be covalently linked to a replacement enzyme (depicted as, but not limited to, GAA) to form the multidomain therapeutic protein (FIGS. 1C and 1D), a replacement enzyme (depicted as, but not limited to, GAA) covalently linked to the C-terminus of each of an anti-internalizing effector-specific half-body, which serves as a first delivery domain, and an internalizing effector-specific scFv-Fc fusion, which serves as a second delivery domain, where both the anti-internalizing effector-specific half-body and associate together to form the multidomain therapeutic protein (FIG. 1D), and a replacement enzyme covalently linked to a first scFv, which is linked, e.g., via a linker, to a second scFv (FIG. 1F).

FIG. 1B provides nonlimiting exemplary illustrations of AAV gene therapy vectors that each encode a multidomain therapeutic protein represented in panel G of FIG. 1A, wherein the scFv is an anti-human CD63 scFv and the replacement enzyme is GAA (e.g., anti-hCD63scFv::hGAA; see, e.g., the amino acid sequence set forth as SEQ ID NO:10). Amino acids 1-117 of SEQ ID NO: 10 provide the amino acid sequence of the heavy chain variable domain (VH) of the H4H12450N antibody; amino acids 118-132 of SEQ ID NO:10 provide an amino acid linker sequence between the heavy and light chain variable domains of H4H12450N; amino acids 133-240 of SEQ ID NO:10 provide the amino acid sequence of the light chain variable domain (VL) of the H4H12450N antibody; amino acids 241-245 of SEQ ID NO:10 provides an amino acid linker sequence between the anti-hCD63scFv and GAA; and amino acids 246-1128 of SEQ ID NO:10 provides the amino acid sequence of the replacement enzyme GAA, or biologically active portion thereof. Exemplary 5′ITR and 3′ ITR sequences are respectively set forth as SEQ ID NO:6 and SEQ ID NO:7. Panel A of this Figure provides an exemplary vector for liver specific expression comprising an exemplary liver specific enhancer (e.g., but not limited to, Serpina1; set forth as SEQ ID NO:9), an exemplary liver specific promoter (e.g., but not limited to, TTR; set forth as SEQ ID NO: 8), an exemplary signal peptide; a nucleic acid sequence encoding the anti-hCD63scFv::hGAA multitherapeutic domain (SEQ ID NO:10), and a poly-A tail. Panel B of this figure provides an exemplary vector similar to that shown in Panel A with an exemplary ubiquitous promoter in place of the liver-specific enhancer and liver-specific promoter sequences. Panel C of this figure provides an exemplary vector similar to that shown in Panel A with an exemplary neuron-specific promoter in place of the liver-specific enhancer (e.g., SerpinA1) and promoter (e.g., TTR). Panel D of this figure provides an exemplary vector similar to that shown in Panel A with an exemplary neuron-specific promoter in combination with a liver-specific enhancer (e.g., SerpinA1) and promoter (e.g., TTR).

FIG. 1C provides nonlimiting exemplary illustrations of expression vectors that each encode a multidomain therapeutic protein as depicted, wherein the half-body is an anti-CD63 antibody, the scFv is an anti-human transferrin receptor scFv, and wherein the replacement enzyme is GAA (e.g., anti-hTfRscFv::hGAA).

FIG. 1D provides nonlimiting exemplary illustrations of expression vectors that each encode a multidomain therapeutic protein as depicted, wherein the half-body is an anti-CD63 antibody, wherein the scFv is an anti-human transferrin receptor (TfR) scFv and the Fc domain is a human IgG4 Fc, and wherein the replacement enzyme is GAA (e.g., anti-hTFRCscFv:hGAA).

FIG. 1E provides nonlimiting exemplary illustrations of expression vectors that each encode a multidomain therapeutic protein represented in Panel H of FIG. 1A, wherein one of the two scFv is an anti-human CD63 scFv, the other of the two scFv is an anti-human transferrin receptor (TfR) scFv, and the replacement enzyme is GAA (e.g., anti-hCD63scFv::hGAA::anti-TfRscFV).

FIG. 1F provides nonlimiting exemplary illustrations of expression vectors that each encode a multidomain therapeutic protein as depicted, wherein one of the two scFv is an anti-human CD63 scFv, the other of the two scFv is an anti-human transferrin receptor (TfR) scFv, and the replacement enzyme is GAA (e.g., anti-hCD63scFv::anti-TfRscFV::GAA or anti-TfRscFV::anti-hCD63scFv::GAA).

FIG. 1G provides nonlimiting exemplary illustrations of expression vectors that each encode a multidomain therapeutic protein as depicted in panel G of FIG. 1A, wherein the scFv is an anti-human transferrin receptor (TfR) scFv and the replacement enzyme is GAA (e.g., anti-TfRscFV:GAA).

FIG. 2 provides RT-qPCR quantification of hGAA-containing RNA expression of liver from mice treated with AAV8 expressing indicated constructs under the mouse TTR promoter, dosed at 4e11 vg/kg. Mice were harvested 4 weeks post-injection.

FIG. 3 provides a western blot of tissues from Gaa^{-/-} mice treated with AAV8 expressing either GAA, αCD63scfv:GAA, or αTFRCscfv:GAA under the TTR promoter at dose of 4e11 vg/kg. Blot was probed for hGAA. Each lane is an individual mouse. Mice were harvested 4 weeks post-injection.

FIG. 4 provides quantification of GAA in serum—see western blot in FIG. 3. Quantification is arbitrary units.

FIG. 5 provides quantification of GAA in cerebrum—see western blot in FIG. 3. Quantification is arbitrary units.

FIG. 6 provides quantification of glycogen in cerebrum, cerebellum, and spinal cord from mice treated with AAV8 expressing GAA, αCD63scfvGAA, or αTFRCscfv:GAA under the TTR promoter at a dose of 4e11 vg/kg. Mouse tissues were collected 4 weeks post-injection.

FIG. 7 provides quantification of glycogen in heart, EDL, soleus, and gastrocnemius muscles from mice treated with AAV8 expressing GAA, αCD63scfvGAA, or αTFRCscfv:GAA under the TTR promoter at a dose of 4e11 vg/kg. Mouse tissues were collected 4 weeks post-injection.

FIG. 8 provides qPCR quantification of anti-TFRCscFv:GAA DNA in liver from Gaa−/− mice treated with AAV8 expressing αTFRCscfv:GAA under the mouse TTR promoter, at indicated doses. Mouse tissues were collected 4 weeks post-injection.

FIG. 9 provides a western blot of indicated tissues from Gaa−/− mice treated with AAV8 expressing αTFRCscfv:GAA under the mouse TTR promoter, at indicated doses. Blot probed for hGAA. Mouse tissues were collected 4 weeks post-injection.

FIG. 10 provides quantification of glycogen in cerebrum and cerebellum from Gaa−/− mice treated with AAV8 expressing αTFRCscfv:GAA under the TTR promoter at indicated doses. Mouse tissues were collected 4 weeks post-injection.

FIG. 11 provides quantification of glycogen in heart, EDL, soleus, and quadricep muscles from Gaa−/− mice treated with AAV8 expressing αTFRCscfv:GAA under the TTR promoter at indicated doses. Mouse tissues were collected 4 weeks post-injection.

FIG. 12 provides immunofluorescence staining of brain sections from Gaa−/− mice treated with AAV8 expressing αTFRCscfv:GAA under the TTR promoter. Mice were treated at a dose of 3.25e12 vg/kg and tissues were collected 4 weeks post-injection. Sections are stained with anti-hGAA antibody, and costained for endothelial cell marker ZO-1, neuron marker NeuN, or oligodendrocyte marker Olig2.

FIG. 13 provides a diagram of transcytosis across the blood-brain barrier (BBB), delivering αTFRCscfv:GAA to the brain. The αTFRCscfv binds the transferrin receptor on the apical (blood) side of the BBB endothelial cell, enters the transcytotic vesicles taking advantage of the recycling of TFRC, and is released on the basal (brain) side.

FIG. 14 provides a simplified diagram of αTFRCscfv:GAA fusion protein. The scfv and human GAA are separated by a 2×G4S linker. The complete nucleotide sequence of the AAV plasmid, AAV8 αTFRCscfv:GAA, is provided as SEQ ID NO:14. (Annotations to SEQ ID NO:14, with nucleotide positions: left ITR—1-141; liver enhancer (mouse serpin A1)—162-233; mouse TTR promoter—246-469; mROR signal peptide—529-615; 8D3scfv (VH-3×G4S-VK)—616-1335; 2×G4S linker—1336-1365; hGAA (amino acids 70-952)—1366-4014; sv40 polyA—4026-4255; right ITR—4278-4418).

FIG. 15 provides quantification of GAA activity of purified hGAA protein (purchased from R&D Systems), and in-house purified αTFRCscfv:GAA.

FIGS. 16A-16D provide immunofluorescence staining of (A) liver, (B) hippocampus, (C) heart, and (D) quadriceps from untreated wildtype (WT) mice, untreated Gaa−/− mice, or Gaa−/− mice treated with AAV8 expressing anti-TFRCscfv:GAA under the TTR promoter. Mice were treated at a dose of 3.25e12 vg/kg and tissues were collected 4 weeks post-injection. Sections are stained with anti-Lamp1 antibody, anti-hGAA antibody, anti-rat Alexa568, and anti-mouse Alexa488, and mounted in Fluoromount-G with DAPI.

FIGS. 17A-17B provides quantification of total LAMP1+ area (μm²) of (A) hippocampus or (B) striated muscle (heart or quadricep) tissue isolated from untreated wildtype (WT) mice, untreated Gaa−/− mice, or Gaa−/− mice treated with AAV8 expressing anti-TFRCscfv:GAA under the TTR promoter. LAMP1 is quantified from 3-8 images/group with ImageJ (particle analysis); Area Fraction=LAMP⁺ Area/Total Area; Integrated Density=Mean Density×Total LAMP⁺ Area; *p<0.05, **p<0.01, ***p<0.001.

### DESCRIPTION

This invention is not limited to particular embodiments, compositions, methods, and experimental conditions described, as such embodiments, compositions, methods, and conditions may vary. The terminology used herein is for the purpose of describing particular embodiments only, and is not intended to be limiting, since the scope of the present invention will be limited only by the appended claims.

Although any methods and materials similar or equivalent to those described herein can be used in the practice or testing of the present invention, some preferred methods and materials are now described. All publications cited herein are incorporated herein by reference to describe in their entirety. Unless defined otherwise, all technical and scientific terms used herein have the same meaning as commonly understood by one of ordinary skill in the art to which this invention belongs.

“Blood-brain barrier” (BBB) refers to the semipermeable membrane barrier that separates the blood from the brain and extracellular fluid in the central nervous system. The barrier blocks passage of, or selectively transports, certain substances to the brain and spinal cord. The blood-brain barrier is formed by brain endothelial cells.

“Therapeutically effective amount” refers to an amount or dosage of the vector delivered to a subject such that the subject achieves a consistent blood level (serum/plasma level) of the encoded therapeutic protein. Generally, concentrations of from about 1×10⁹ to about 1×10¹⁶ genomes vector may be utilized in the method. The dosage for delivery to liver may be about 1×10¹⁰ to 5×10¹³ AAV genomes per kg. The dosage will be adjusted to balance the therapeutic benefit of crossing the blood brain barrier to achieve the desired effect of the molecule against any side effects and such dosages may vary depending upon the recombinant vector that is employed. The levels of expression of the transgene can be monitored in the blood circulation by extraction of serum or plasma to determine the frequency of dosage of vectors that will achieve a steady state of circulating protein. One skilled in the art can determine specific values for an effective amount by, for example, performing experiments to determine consistent blood levels of therapeutic protein over consecutive days, weeks, or months following vector delivery. Suitable experiments to test for circulating therapeutic protein are known in the art, including but not limited to western blot, ELISA, LC-MS, etc. In one embodiment, a therapeutically effective amount of scFv-GAA fusion protein in the CNS is an amount of viral vector that produces sufficient amounts of scFv-GAA fusion protein to reduce stored glycogen in CNS tissue, for example in spinal cord, cerebellum, or hippocampus tissue. See also, International Publication No. WO 2019/157224 (which reference is incorporated herein in its entirety by reference).

### CNS Disorders

Various brain disorders may benefit from the mode of delivery of therapeutic proteins described herein. CNS disorders and disorders with neurological symptoms amenable to protein therapies include, but are not limited to: Alzheimer's disease, brain cancer, Behcet's disease, cerebral lupus, Creutzfeldt-Jakob disease, dementia, epilepsy, encephalitis, Friedreich's ataxia, Guillain-Barre syndrome, Gaucher's disease, headache, hydrocephalus, Huntington's disease, intracranial hypertension, leukodystrophy, migraine, myasthenia gravis, muscular dystrophy, multiple sclerosis, narcolepsy, neuropathy, Prader-Willi syndrome, Parkinson's disease, Rett syndrome, restless leg syndrome, sleep disorders, subarachnoid hemorrhage, stroke, traumatic brain injury, trigeminal neuralgia, transient ischemic attack, and Von Hippel-Lindau syndrome (angiomatosis).

Anti-CD63-fusion delivery of a therapeutic protein to the CNS may be particularly beneficial due to its ubiquitous expression, its role as a membrane protein of extracellular vesicles (EVs; e.g., exosomes) and association with integrins. Other cell-surface receptors with similar properties as internalizing effectors may be tissue or cell-type specific in order to enhance the desired location of the uptake, as discussed throughout the specification.

Anti-transferrin receptor (TfR or TFR)-fusion delivery of a therapeutic protein to the CNS may be particularly beneficial. Trafficking and delivery of therapeutic proteins therefore will be enhanced by the use of delivery mechanisms, such as anti-transferrin receptor-fusion to particular blood-brain-barrier (BBB) targets. Some BBB targets have been shown as beneficial for CNS uptake (Zuchero, et al., 6 Jan. 2016, Neuron, 89(1): 70-82; Boado, R J et al, Mol Pharm. 2014 Aug. 4; 11(8): 2928-2934; each of which reference is incorporated herein in its entirety by reference). Other cell-surface receptors with similar BBB uptake properties to transferrin receptors include, but are not limited to: insulin receptor, CD98, and Basigin (Bsg).

In some embodiments, the targeted delivery of the therapeutic protein to CNS tissue (e.g., brain) is employed by use of anti-transferrin receptor or anti-insulin receptor, or anti-CD98 or anti-Bsg. The therapeutic protein may also be fused to another internalizing effector antibody.

### Lysosomal Storage Diseases

“Enzyme-deficiency diseases” include nonlysosomal storage disease such as Krabbe disease (galactosylceramidase), phenylketonuria, galactosemia, maple syrup urine disease, mitochondrial disorders, Friedreich ataxia, Zellweger syndrome, adrenoleukodystrophy, Wilson disease, hemochromatosis, ornithine transcarbamylase deficiency, methylmalonic academia, propionic academia, and lysosomal storage diseases. “Lysosomal storage diseases” include any disorder resulting from a defect in lysosome function. Currently, approximately 50 lysosomal storage disorders have been identified, the most well-known of which include Tay-Sachs, Gaucher's, and Niemann-Pick diseases. The pathogeneses of the diseases are ascribed to the buildup of incomplete degradation products in the lysosome, usually due to loss of protein function. Lysosomal storage diseases are caused by loss-of-function or attenuating variants in the proteins whose normal function is to degrade or coordinate degradation of lysosomal contents. The proteins affiliated with lysosomal storage diseases include enzymes, receptors, and other transmembrane proteins (e.g., NPC1), post-translational modifying proteins (e.g., sulfatase), membrane transport proteins, and nonenzymatic cofactors and other soluble proteins (e.g., GM2 ganglioside activator). Thus, lysosomal storage diseases encompass more than those disorders caused by defective enzymes per se, and include any disorder caused by any molecular defect. Thus, as used herein, the term “enzyme” is meant to encompass those other proteins associated with lysosomal storage diseases.

The nature of the molecular lesion affects the severity of the disease in many cases, i.e., complete loss-of-function tends to be associated with pre-natal or neo-natal onset and involves severe symptoms; partial loss-of-function is associated with milder (relatively) and later-onset disease. Generally, only a small percentage of activity needs to be restored to have to correct metabolic defects in deficient cells. Table 1 lists some of the more common lysosomal storage diseases and their associated loss-of-function proteins. Lysosomal storage diseases are generally described in Desnick and Schuchman, 2012.

Lysosomal storage diseases are a class of rare diseases that affect the degradation of myriad substrates in the lysosome. Those substrates include sphingolipids, mucopolysaccharides, glycoproteins, glycogen, and oligosaccharides, which can accumulate in the cells of those with disease leading to cell death. Organs affected by lysosomal storage diseases include the central nervous system (CNS), the peripheral nervous system (PNS), lungs, liver, bone, skeletal and cardiac muscle, and the reticuloendothelial system.

Options for the treatment of lysosomal storage diseases include enzyme replacement therapy (ERT), substrate reduction therapy, pharmacological chaperone-mediated therapy, hematopoietic stem cell transplant therapy, and gene therapy. An example of substrate reduction therapy includes the use of MIGLUSTAT or ELIGLUSTAT to treat Gaucher's Type 1. These drugs act by blocking synthase activity, which reduces subsequent substrate production. Hematopoietic stem cell therapy (HSCT), for example, is used to ameliorate and slow down the negative central nervous system phenotype in patients with some forms of mucopolysaccharidosis (MPS). See R. M. Boustany, “Lysosomal storage diseases—the horizon expands,” 9(10) Nat. Rev. Neurol. 583-98, October 2013; which reference is incorporated herein in its entirety by reference. Table 1 lists some lysosomal storage diseases and their associated enzymes or other proteins.

TABLE 1
Lysosomal Storage Diseases

| Class | Disease | Involved Enzyme/Protein |
|---|---|---|
| Sphingolipidoses | Fabry disease | α-Galactosidase A |
| | Farber lipogranulomatosis | Ceramidase |
| | Gaucher disease type I | β-Glucosidase |
| | Gaucher disease types II and III | Saposin-C activator |
| | Niemann-Pick diseases types A and B | Sphingomyelinase |
| | GM1-gangliosidosis | β-Galactosidase |
| | GM2-gangliosidosis (Sandhoff) | β-Hexosaminidase A and B |
| | GM2-gangliosidosis (Tay-Sachs) | β-Hexosaminidase A |
| | GM2-gangliosidosis (GM2-activator | GM2-activator protein |
| | deficiency) | |
| | GM3-gangliosidosis | GM3 synthase |
| | Metachromatic leukodystrophy | Arylsulfatase A |
| | Sphingolipid-activator deficiency | Sphingolipid activator |
| Mucopolysaccharidoses | MPS I (Scheie, Hurler-Scheie, and Hurler | α-Iduronidase |
| | disease) | |
| | MPS II (Hunter) | Iduronidase-2-sulphatase |
| | MPS IIIA (Sanfilippo A) | Heparan N-sulphatase |
| | MPS IIIB (Sanfilippo B) | N-acetyl-α-glucosaminidase |
| | MPS IIIC (Sanfilippo C) | Acetyl-CoA; α-glucosamide |
| | | N-acetyltransferase |
| | MPS IIID (Sanfilippo D) | N-acetylglucosamine-6- |
| | | sulphatase |
| | MPS IVA (Morquio syndrome A) | N-acetylgalactosamine-6- |
| | | sulphate sulphatase |
| | MPS IVB (Morquio syndrome B) | β-Galactosidase |
| | MPS VI (Maroteaux-Lamy) | N-acetylgalactosamine-4- |
| | | sulphatase (arylsulphatase B) |
| | MPS VII (Sly disease) | β-Glucuronidase |
| | MPS IX | Hylauronidase |
| Glycogen storage | Pompe (glycogen storage disease type II) | α-Glucosidase 2 |
| disease | | |
| Lipid | Lysosomal acid lipase deficiency (LAL-D; | Lysosomal acid lipase |
| metabolism | Wolman disease) | |

Two of the most common LSDs are Pompe disease and Fabry disease. Pompe disease, which has an estimated incidence of 1 in 10,000, is caused by defective lysosomal enzyme alpha-glucosidase (GAA), which results in the deficient processing of lysosomal glycogen. Accumulation of lysosomal glycogen occurs predominantly in skeletal, cardiac, and hepatic tissues. Infantile-onset Pompe causes cardiomegaly, hypotonia, hepatomegaly, and death due to cardiorespiratory failure, usually before two years of age. Adult onset Pompe occurs as late as the second to sixth decade and usually involves only skeletal muscle. Treatments currently available include Genzyme's MYOZYME®/LUMIZYME® (alglucosidase alfa), which is a recombinant human alpha-glucosidase produced in CHO cells and administered by intravenous infusion.

Fabry disease, including mild/late onset cases, has an overall estimated incidence of 1 in 3,000; it is caused by defective lysosomal enzyme alpha-galactosidase A (GLA), which results in the accumulation of globotriaosylceramide within the blood vessels and other tissues and organs. Symptoms associated with Fabry disease include pain from nerve damage and/or small vascular obstruction, renal insufficiency and eventual failure, cardiac complications such as high blood pressure and cardiomyopathy, dermatological symptoms such as formation of angiokeratomas, anhidrosis or hyperhidrosis, and ocular problems such as cornea verticillata, spoke-like cataract, and conjunctival and retinal vascular abnormalities. Treatments currently available include Genzyme's FABRAZYME® (agalsidase beta), which is a recombinant human alpha-galactosidase A produced in CHO cells and administered by intravenous infusion; Shire's REPLAGAL™ (agalsidase alfa), which is a recombinant human alpha-galactosidase A produced in human fibroblast cells and administered by intravenous infusion; and Amicus's GALAFOLD™ (migalastat or 1-deoxygalactonojirimycin), an orally administered small molecule chaperone that shifts the folding of abnormal alpha-galactosidase A to a functional conformation.

Current treatments for lysosomal storage diseases are less than optimal. For example, ERT generally must be administered at a high frequency and a high dose, such as biweekly and up to 40 mg/kg. Also, some replaced enzymes can be immunologically cross-reactive (CRIM), stimulating production of IgG in the subject and thus hindering delivery of the enzyme to the lysosome via the mannose-6-phosphate (M6P) receptor. IgGs may shield the M6P residues of the replacement enzyme, and the antigen-IgG-antibody complex may be taken up into cellular lysosomes via the Fc receptor, thereby shunting the replacement enzyme preferentially to macrophages.

Delivery of replacement enzymes to the appropriate affected tissues is also inefficient (see Table 2 and Desnick & Schuchman, “Enzyme replacement therapy for lysosomal diseases: lessons from 20 years of experience and remaining challenges,” 13 Annu. Rev. Genomics Hum. Genet. 307-35, 2012, which is incorporated herein in its entirety by reference). For example, patients undergoing long-term enzyme replacement therapy for infantile Pompe can still suffer from hypernasal speech, residual muscle weakness, ptosis, osteopenia, hearing loss, risk for aspiration, dysphagia, cardiac arrhythmia, and difficulty swallowing. Doses of replacement enzyme often must be increased over time to 40 mg/kg weekly or biweekly.

TABLE 2
Inefficient tissue targeting of ERT

| | | Easy to reach | Hard to reach |
|---|---|---|---|
| Disease | Subtype(s) | tissue | tissue |
| Gaucher disease | Type 1 | Spleen, liver, | Bone |
| | | bone marrow | |
| | Types 2 and | Spleen, liver, | Bone, brain |
| | 3 | bone marrow | |
| Fabry disease | Classic and | Vascular | Kidney, heart |
| | late onset | endothelium | |
| Mucopoly- | All | Spleen, liver, | Bone, brain, |
| saccharidoses | | bone marrow | cartilage |
| α-Mannosidosis | — | Spleen, liver, | Bone, brain |
| | | bone marrow | |
| Niemann-Pick | Type B | Spleen, liver, | Alveolar |
| disease | | bone marrow | macrophages |
| Pompe disease | Infantile | — | Heart, smooth and |
| | | | skeletal muscle |
| | Later onset | — | Smooth muscle |
| | | | and respiratory |
| | | | skeletal muscle |

Endogenous mannose-6 phosphate receptor (MPR) mediates the transport of most recombinant enzymes to the lysosome. Two complementary forms of MPR exist: cation-independent (CI-MPR), and cation-dependent (CD-MPR). Knockouts of either form have missorted lysosomal enzymes. Lysosomal hydrolases are synthesized in the endoplasmic reticulum and move to the cis-Golgi network, where they are covalently modified by the addition of mannose-6-phosphate (M6P) groups. The formation of this marker depends on the sequential effect of two lysosomal enzymes: UDP-N-acetylglucosamine-l-phosphotransferase (G1cNac-phosphotransferase) and N-acetylglucosamine-l-phosphodiester-α-N-acetyl-glucosaminidase (uncovering enzyme). GlcNac-phosphotransferase catalyzes the transfer of a G1cNAc-1-phosphate residue from UDP-G1cNAc to C6 positions of selected mannoses in high-mannose type oligosaccharides of the hydrolases. Then, the uncovering enzyme removes the terminal G1cNAc, exposing the M6P recognition signal. At the trans-Golgi network, the M6P signal allows the segregation of lysosomal hydrolases from all other types of proteins through selective binding to the M6P receptors. The clathrin-coated vesicles produced bud off from the trans-Golgi network and fuse with late endosomes. At the low pH of the late endosome, the hydrolases dissociate from the M6P receptors, and the empty receptors are recycled to the Golgi apparatus for further rounds of transport.

With the exception of β-glucocerebrosidase, which is delivered via the mannose receptor, recombinant lysosomal enzymes comprise M6P glycosylation and are delivered to the lysosome primarily via CI-MPR/IGF2R. Glycosylation/CI-MPR-mediated enzyme replacement delivery, however, does not reach all clinically relevant tissues (Table 2). Improvement to enzyme replacement therapy has centered on improving CI-MPR delivery by (i) increasing surface expression of CI-MPR using the 02-agonist clenbuterol (Koeberl et al., “Enhanced efficacy of enzyme replacement therapy in Pompe disease through mannose-6-phosphate receptor expression in skeletal muscle,” 103(2) Mol. Genet. Metab. 107-12, 2011); (ii) increasing the amount of M6P residues on enzyme (Zhu et al., “Conjugation of mannose-6-phosphate-containing oligosaccharides to acid alpha-glucosidase improves the clearance of glycogen in Pompe mice,” 279(48) J. Biol. Chem. 50336-41, 2004); or (iii) fusing an IGF-II domain to the enzyme (Maga et al., “Glycosylation-independent lysosomal targeting of acid alpha-glucosidase enhances muscle glycogen clearance in Pompe mice,” 288(3) J. Biol. Chem. 1428-38, 2013) (all preceding references are incorporated herein in their entireties by reference).

A large number of lysosomal storage diseases are inadequately treated by enzyme replacement therapy or gene therapy mainly due to poor targeting of the replacement enzyme to the relevant tissue or organ, negative immunological reactions in the recipient host, and low serum half-life. A need exists for improved enzyme replacement therapies that enhance and promote better tissue biodistribution and lysosomal uptake of the enzyme, especially in the brain and spinal cord without undesirable intrathecal injections. Applicants have developed an improved enzyme replacement therapy using CI-MPR independent binding protein-guided delivery of enzymes and liver expression to provide enzyme to the lysosome of target-affected tissues, particularly CNS tissues.

Lysosomal storage diseases can be categorized according to the type of product that accumulates within the defective lysosome. Sphingolipidoses are a class of diseases that affect the metabolism of sphingolipids, which are lipids containing fatty acids linked to aliphatic amino alcohols (reviewed in S. Hakomori, “Glycosphingolipids in Cellular Interaction, Differentiation, and Oncogenesis,” 50 Annual Review of Biochemistry 733-764, July 1981; which reference is incorporated herein in its entirety by reference). The accumulated products of sphingolipidoses include gangliosides (e.g., Tay-Sachs disease), glycolipids (e.g., Fabry disease), and glucocerebrosides (e.g., Gaucher's disease).

Mucopolysaccharidoses are a group of diseases that affect the metabolism of glycosaminoglycans (GAGS or mucopolysaccharides), which are long unbranched chains of repeating disaccharides that help build bone, cartilage, tendons, corneas, skin, and connective tissue (reviewed in J. Muenzer, “Early initiation of enzyme replacement therapy for the mucopolysaccharidoses,” 111(2) Mol. Genet. Metab. 63-72 (February 2014); Sasisekharan et al., “Glycomics approach to structure-function relationships of glycosaminoglycans,” 8(1) Ann. Rev. Biomed. Eng. 181-231 (December 2014); each of which reference is incorporated herein in its entirety by reference). The accumulated products of mucopolysaccharidoses include heparan sulfate, dermatan sulfate, keratin sulfate, various forms of chondroitin sulfate, and hyaluronic acid. For example, Morquio syndrome A is due to a defect in the lysosomal enzyme galactose-6-sulfate sulfatase, which results in the lysosomal accumulation of keratin sulfate and chondroitin 6-sulfate.

Glycogen storage diseases (a.k.a., glycogenosis) result from a cell's inability to metabolize (make or break-down) glycogen. Glycogen metabolism is moderated by various enzymes or other proteins including glucose-6-phosphatase, acid alpha-glucosidase, glycogen debranching enzyme, glycogen branching enzyme, muscle glycogen phosphorylase, liver glycogen phosphorylase, muscle phosphofructokinase, phosphorylase kinase, glucose transporter, aldolase A, beta-enolase, and glycogen synthase. An exemplar lysosomal storage/glycogen storage disease is Pompe disease, in which defective acid alpha-glucosidase causes glycogen to accumulate in lysosomes. Symptoms include hepatomegaly, muscle weakness, heart failure, and in the case of the infantile variant, death by age 2 (see DiMauro and Spiegel, “Progress and problems in muscle glycogenosis,” 30(2) Acta Myol. 96-102 (October 2011); incorporated herein in its entirety by reference).

“Multidomain therapeutic protein” includes (i) a single protein that contains more than one functional domain, (ii) a protein that contains more than one polypeptide chain, and (iii) a mixture of more than one protein or more than one polypeptide. The term polypeptide is generally taken to mean a single chain of amino acids linked together via peptide bonds. The term protein encompasses the term polypeptide, but also includes more complex structures. That is, a single polypeptide is a protein, and a protein can contain one or more polypeptides associated in a higher order structure. For example, hemoglobin is a protein containing four polypeptides: two alpha globin polypeptides and two beta globin polypeptides. Myoglobin is also a protein, but it contains only a single myoglobin polypeptide.

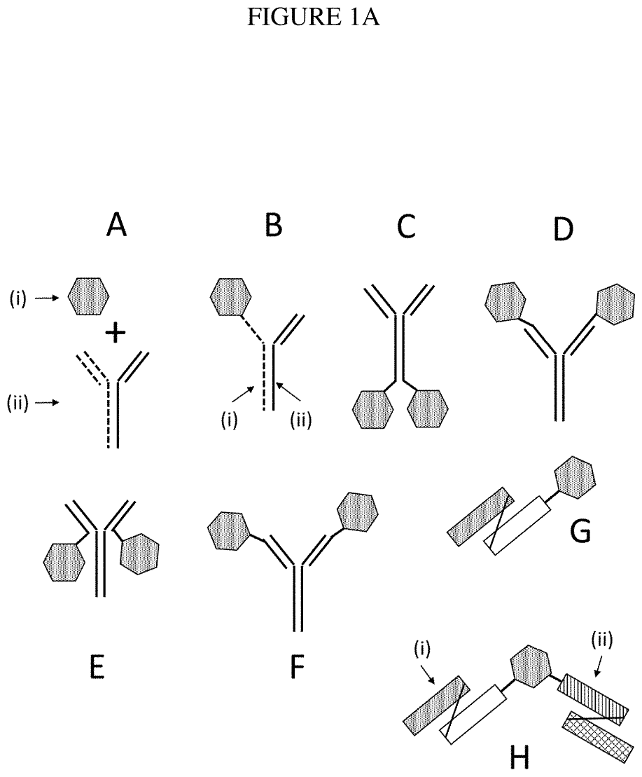

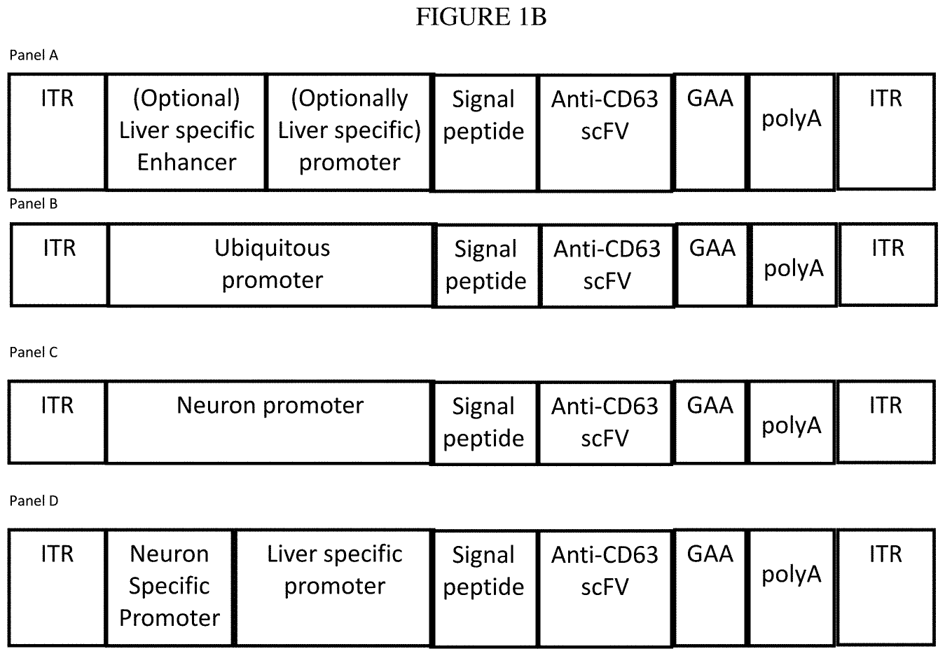

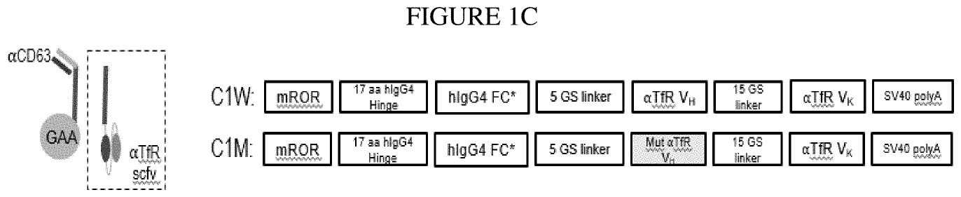

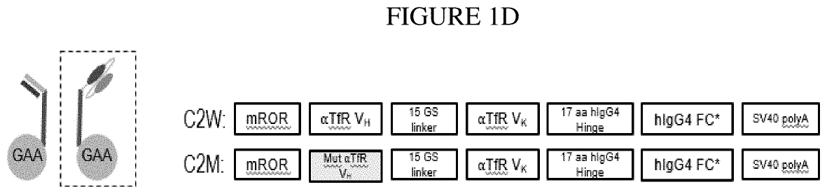

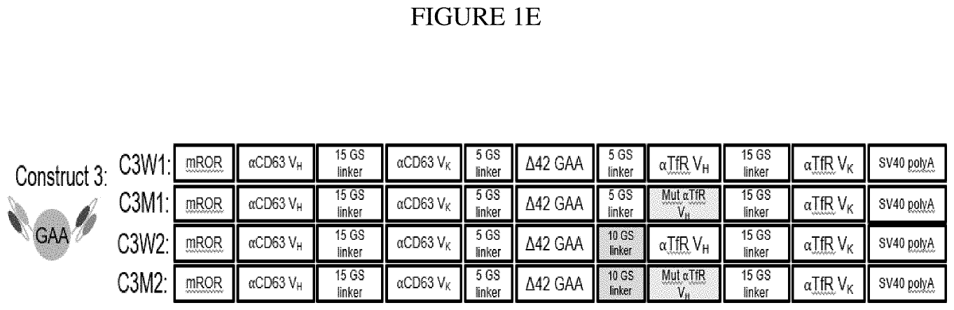

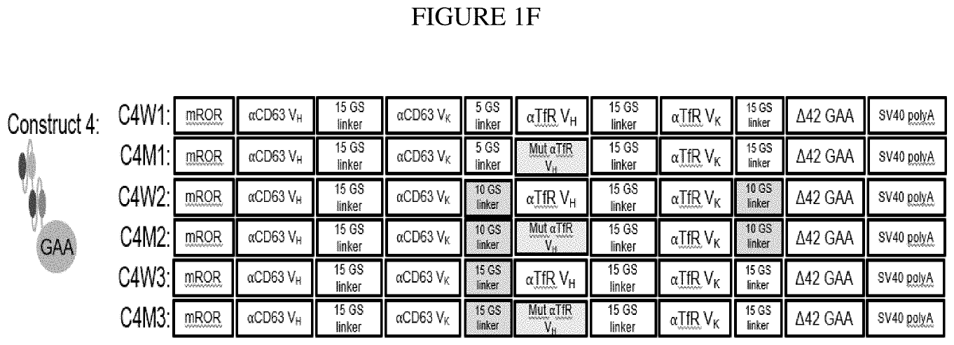

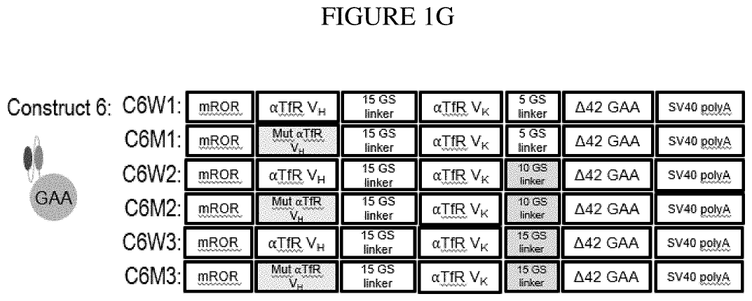

The multidomain therapeutic protein comprises one or more polypeptides and at least two domains providing two functions. One of those domains is the “enzyme domain” which provides the replacement of a defective protein activity associated with an enzyme deficiency disease. The other of those domains is the “delivery domain” which provides binding to an internalizing effector. Thus, a single polypeptide that provides an enzyme replacement activity and the ability to bind to an internalizing effector (a.k.a. internalizing effector-binding protein (delivery domain activity)) is a multidomain therapeutic protein. Also, a mixture of proteins, wherein one protein provides the enzyme function, and another protein provides the internalizing effector binding activity, is a multidomain therapeutic protein. FIG. 1A depicts various exemplars of multidomain therapeutic proteins. In one example (FIG. 1A, panel A), the multidomain therapeutic protein contains an enzyme (represented by the hexagon) and a bispecific antibody (the IE-BP) that binds the enzyme (hashed lines) and an internalizing effector (solid lines). Here, one arm of the bispecific antibody binds noncovalently to the enzyme, and the other arm binds noncovalently to the internalizing effector, thereby enabling the internalization of the replacement enzyme into the cell or subcellular compartment. In another example (panel B), the multidomain therapeutic protein comprises a single protein containing two polypeptides, one polypeptide having enzyme function and the other having delivery domain function. Here, the enzyme is fused to an immunoglobulin Fc domain or heavy chain constant region, which associates with the Fc domain of the enzyme half-antibody to form the bifunctional multidomain therapeutic protein. The embodiment depicted in panel B is similar to that in panel A, except that the enzyme is covalently attached to one of the half-antibodies, rather than through antigen-antibody interaction at the immunoglobulin variable domain of the half-antibody.

In other examples, the multidomain therapeutic protein consists of the enzyme covalently linked (directly or indirectly through a linker) to the delivery domain. In one embodiment, the enzyme is attached to the C-terminus of an immunoglobulin molecule (e.g., the heavy chain or alternatively the light chain). In another embodiment, the enzyme is attached to the N-terminus of the immunoglobulin molecule (e.g., the heavy chain or alternatively the light chain). In these exemplars, the immunoglobulin molecule is the delivery domain. In yet another embodiment, the enzyme is attached to the C-terminus of a scFv molecule that binds the internalizing effector.

In one embodiment, the multidomain therapeutic protein comprises at least two, and in some embodiments no more than two, delivery domains, each of which is directed toward a distinct epitope, either on the same antigen or on two different antigens. In one embodiment, the first delivery domain binds to a lysosomal trafficking molecule, other internalizing effector, or other similar cell-surface receptor. In another embodiment, the second delivery domain binds to a transcytosis effector to facilitate transcellular transport of the multidomain therapeutic protein. In one embodiment, the transcytosis effector is inter alia an LDL receptor, an IgA receptor, a transferrin receptor, or a neonatal Fc receptor (FcRn). In a specific embodiment, the transcytosis delivery domain comprises a molecule that binds to a transferrin receptor, such as an anti-transferrin receptor antibody or an anti-transferrin receptor scFv molecule. Tuma and Hubbard, “Transcytosis: Crossing Cellular Barriers,” Physiological Reviews, 83(3): 871-935 (1 Jul. 2003) is incorporated herein by reference for cell surface receptors that mediate transcytosis that are useful in the practice of the subject invention. In one embodiment, a second delivery domain binds to a transferrin receptor, or other similar cell-surface protein, such as an insulin receptor, CD98, or Basigin (Bsg). Each multidomain therapeutic protein comprising at least two delivery domains also comprises at least one enzyme domain, e.g., each of the at least two delivery domains may or may not be independently associated an enzyme domain in a manner described herein (e.g., via an antigen-antibody interaction, via a direct covalent link, via an indirect covalent link, etc.), wherein at least one of the at least two delivery domains is associated with the enzyme domain. Additionally, each of the at least two delivery domains may independently comprise an antibody, a half-body, or an scFv (e.g., an scFv fused with an Fc).

“Enzyme domain” or “enzyme” denotes any protein associated with the etiology or physiological effect of an enzyme deficiency disease. An enzyme includes the actual enzyme, transport protein, receptor, or other protein that is defective and that is attributed as the molecular lesion that caused the disease. An enzyme also includes any protein that can provide a similar or sufficient biochemical or physiological activity that replaces or circumvents the molecular lesion of the disease. For example, an “isozyme” may be used as an enzyme. Examples of lysosomal storage disease-related proteins include those listed in Table 1 as “Involved Enzyme/Protein” and any known or later discovered protein or other molecule that circumvents the molecular defect of the enzyme-deficiency disease.

In some embodiments, the enzyme is a hydrolase, including esterases, glycosylases, hydrolases that act on ether bonds, peptidases, linear amidases, diphosphatases, ketone hydrolases, halogenases, phosphoamidases, sulfohydrolases, sulfinases, desulfinases, and the like. In some embodiments, the enzyme is a glycosylase, including glycosidases and N-glycosylases. In some embodiments, the enzyme is a glycosidase, including alpha-amylase, beta-amylase, glucan 1,4-alpha-glucosidase, cellulose, endo-1,3(4)-beta-glucanase, inulinase, endo-1,4-beta-xylanase, endo-1,4-b-xylanase, dextranase, chitinase, polygalacturonidase, lysozyme, exo-alpha-sialidase, alpha-glucosidase, beta-glucosidase, alpha-galactosidase, beta-galactosidase, alpha-mannosidase, beta-mannosidase, beta-fructofuranosidase, alpha,alpha-trehalose, beta-glucuronidase, xylan endo-1,3-beta-xylosidase, amylo-alpha-1,6-glucosidase, hyaluronoglucosaminidase, hyaluronoglucuronidase, and the like.

In the case of Pompe disease, in which the molecular defect is a defect in α-glucosidase activity, enzymes include human alpha-glucosidase, and “isozymes” such as other alpha-glucosidases, engineered recombinant alpha-glucosidase, other glucosidases, recombinant glucosidases, any protein engineered to hydrolyze a terminal nonreducing 1-4 linked alpha-glucose residue to release a single alpha-glucose molecule, any EC 3.2.1.20 enzyme, natural or recombinant low pH carbohydrate hydrolases for glycogen or starches, and glucosyl hydrolases such as sucrase isomaltase, maltase glucoamylase, glucosidase II, and neutral alpha-glucosidase.

An “internalizing effector” includes a protein, in some cases a receptor protein, that is capable of being internalized into a cell or that otherwise participates in or contributes to retrograde membrane trafficking. Internalization effector, internalizing effector, internalization receptor, and internalizing receptor are used interchangeably herein. In some instances, the internalizing effector is a protein that undergoes transcytosis; that is, the protein is internalized on one side of a cell and transported to the other side of the cell (e.g., apical-to-basal). In some embodiments, the internalizing effector protein is a cell surface-expressed protein or a soluble extracellular protein. The present invention also contemplates embodiments in which the internalizing effector protein is expressed within an intracellular compartment, such as the endosome, endoplasmic reticulum, Golgi, lysosome, etc. For example, proteins involved in retrograde membrane trafficking (e.g., pathways from early/recycling endosomes to the trans-Golgi network) may serve as internalizing effector proteins in various embodiments of the present invention. In any event, the binding of the delivery domain to an internalizing effector protein causes the entire multidomain therapeutic protein, and any molecules associated therewith (e.g., an enzyme(s)), to also become internalized into the cell. As explained below, internalizing effector proteins include proteins that are directly internalized into a cell, as well as proteins that are indirectly internalized into a cell.

Internalizing effector proteins that are directly internalized into a cell include membrane-associated molecules with at least one extracellular domain (e.g., transmembrane proteins, GPI-anchored proteins, etc.), which undergo cellular internalization, and are preferably processed via an intracellular degradative and/or recycling pathway. Specific nonlimiting examples of internalizing effector proteins that are directly internalized into a cell include: transferrin receptor (TfR), CD63, MHC-I (e.g., HLA-B27), Kremen-1, Kremen-2, LRP5, LRP6, LRP8, LDL-receptor, LDL-related protein 1 receptor, ASGR1, ASGR2, amyloid precursor protein-like protein-2 (APLP2), apelin receptor (APLNR), MAL (Myelin And Lymphocyte protein, a.k.a. VIP17), IGF2R, vacuolar-type H+ ATPase, diphtheria toxin receptor, folate receptor, glutamate receptors, glutathione receptor, leptin receptors, scavenger receptors (e.g., SCARA1-5, SCARB1-3, CD36), and the like.

In one embodiment, the internalizing effector is expressed in several tissue types and is useful in treatment where targeting of both the CNS and a peripheral cell type is desired. Internalizing effectors useful in trafficking to both CNS and peripheral cell types include, but are not limited to TfR, CD63, MHC-I, vacuolar-type H+ ATPase, IGF2R, integrin alpha-7 (ITGA7), LRP5, LRP6, LRP8, Kremen-2, LDL receptor, LDL-related protein 1 receptor, amyloid precursor protein-like protein-2 (APLP2), apelin receptor (APLNR), PRLR, MAL (myelin and lymphocyte protein (MAL), diphtheria toxin receptors, HBEGF (heparin binding EGF like growth factor), glutathione receptors, glutamate receptors, leptin receptors, and folate receptors. In certain embodiments, the internalizing effector is prolactin receptor (PRLR). It was discovered that PRLR is not only a target for certain therapeutic applications, but also an effective internalizing effector protein on the basis of its high rate of internalization and turn-over.

Targeting internalizing effectors expressed by several cell types may be useful where targeting of both the CNS and a peripheral cell type is desired, e.g., in treating diseases such as Fabry disease, Gaucher's disease, MPS I, MPS II, MPS IIIA, MIPS IIIB, MPS HID, MIPS IVB, MPS VI, MPS VII, MPS IX, Pompe disease, lysosomal acid lipase deficiency, metachromatic leukodystrophy, Niemann-Pick diseases types A, B, and C2, alpha mannosidosis, neuraminidase deficiency, sialidosis, aspartylglycosaminuria, combined saposin deficiency, atypical Gaucher's disease, Farber lipogranulomatosis, fucosidosis, and beta mannosidosis.

In another embodiment, the internalizing effector is expressed in a few tissue types. In one example, the internalizing effector may target bone and cartilage preferentially. Effectors useful in trafficking to CNS, and to either or both bone and cartilage include, but are not limited to collagen X, integrin alpha 10 (ITGA10), fibroblast growth factor receptor 3 (FGFR3), fibroblast growth factor receptor isoform C (FGFR3C), hyaluronan and proteoglycan link protein 1 (CRTL1), aggrecan, collagen II, and Kremen-1. Such effectors are useful in treatment where targeting of both the CNS and the skeleton and cartilage is desired.

Targeting internalizing effectors preferentially expressed by bone and cartilage may be useful where targeting both the CNS and the skeleton and cartilage is desired, e.g., in treating diseases such as MPS I, MPS II, MPS IIIA, MPS IIIB, MPS IIID, MPS IVA, MPS IVB, MPS VI, MPS VII, MPS IX, beta mannosidosis, Gaucher's disease, atypical Gaucher's disease, combined saposin deficiency, aspartylglycosaminuria, Farber lipogranulomatosis, sialidosis, neuraminidase deficiency, mucopolysaccharidoses, and alpha mannosidosis.

In yet another embodiment, the internalizing effector is expressed preferentially in a particular tissue or cell type, such as macrophages, monocytes, and microglia. Effectors useful in trafficking to CNS, and to macrophages include, but are not limited to, scavenger receptor A1-5 (SCARA1-5), SCARB1-3, CD36, MSR1 (macrophage scavenger receptor 1), MRC1 (macrophage mannose receptor 1), VSIG4 (V-set and immunoglobulin domain-containing protein 4), CD68 (macrosialin), and CSF1R (macrophage colony-stimulating factor 1 receptor). Such effectors are useful in treatment where targeting of both the CNS and macrophages is desired. CNS macrophages may be referred to as microglia.

Targeting internalizing effectors expressed preferentially by macrophages (monocytes or microglia) may be useful where targeting both CNS and macrophages (or microglia) is desired, e.g., in treating diseases such as lysosomal acid lipase deficiency, Gaucher's disease, atypical Gaucher's disease, combined saposin deficiency, and Farber lipogranulomatosis.

In certain embodiments, the internalizing effector is a kidney specific internalizing effector, such as CDH16 (Cadheri-16), CLDN16 (Claudn-16), KL (Klotho), PTH1R (parathyroid hormone receptor), SLC22A13 (Solute carrier family 22 member 13), SLC5A2 (Sodium/glucose cotransporter 2), and UMOD (Uromodulin).

Targeting internalizing effectors preferentially expressed in the kidney may be useful where targeting both the CNS and the kidney is desired, e.g., in treating disease such as Fabry disease, Alport syndrome, polycystic kidney disease, and thrombotic thrombocytopenic purpura.

In yet another embodiment, the internalizing effector is expressed preferentially in a particular tissue or cell type, such as the liver. Effectors useful in trafficking to CNS, and to liver include, but are not limited to, ASGR1 and ASGR2. Such effectors are useful in treatment where targeting of both the CNS and liver is desired.

Targeting internalizing effectors expressed preferentially in the liver may be useful where targeting both CNS and liver is desired, e.g., in treating diseases such as lysosomal acid lipase deficiency, Gaucher's disease, MPS VI, MPS VII, MPS II, Niemann-Pick disease types A, B, and C2, sialidosis, neuraminidase deficiency, atypical Gaucher disease, combined saposin deficiency, and Farber lipogranulomatosis.

In some embodiments, the internalizing effector is a muscle-specific internalizer, such as BMPR1A (bone morphogenetic protein receptor 1A), m-cadherin, CD9, MuSK (muscle-specific kinase), LGR4/GPR48 (G protein-coupled receptor 48), cholinergic receptor (nicotinic) alpha 1, CDH15 (Cadheri-15), ITGA7 (integrin alpha-7), CACNG1 (L-type calcium channel subunit gamma-1), CACNA15 (L-type calcium channel subunit alpha-15), CACNG6 (L-type calcium channel subunit gamma-6), SCN1B (Sodium channel subunit beta-1), CHRNA1 (ACh receptor subunit alpha), CHRND (ACh receptor subunit delta), LRRC14B (leucine-rich repeat-containing protein 14B), dystroglycan (DAG1), and POPDC3 (Popeye domain-containing protein 3).

Targeting internalizing effectors expressed preferentially by muscle may be useful where targeting both the CNS and muscle tissue is desired, e.g., in treating a disease such as Pompe disease.

In some embodiments, the internalizing effector is ITGA7, ITGA10, CD9, CD63, ALPL2, MSR1, ASGR1, ASGR2, or PRLR. Antibodies to ITGA7, ITGA10, CD9, CD63, APLP2, MSR1, ASGR1, ASGR2 or PRLR are well-known in the art. A skilled artisan could readily link these well-known antibodies, or antigen binding portions thereof (e.g., scFv derived therefrom) to a therapeutic protein as described herein to make and use a multidomain therapeutic protein as described herein.

In those embodiments in which the internalizing effector (IE) is directly internalized into a cell, the delivery domain can be, e.g., an antibody or antigen-binding fragment of an antibody that specifically binds the IE, or a ligand or portion of a ligand that specifically interacts with the IE. For example, if the IE is Kremen-1 or Kremen-2, the delivery domain can comprise or consist of a Kremen ligand (e.g., DKK1) or Kremen-binding portion thereof. As another example, if the IE is a receptor molecule such as ASGR1, the delivery domain can comprise or consist of a ligand specific for the receptor (e.g., asialoorosomucoid (ASOR) or Beta-Ga1NAc) or a receptor-binding portion thereof.

Internalizing effector proteins that are indirectly internalized into a cell include proteins and polypeptides that do not internalize on their own but become internalized into a cell after binding to or otherwise associating with a second protein or polypeptide that is directly internalized into the cell. Proteins that are indirectly internalized into a cell include, e.g., soluble ligands that are capable of binding to an internalizing cell surface-expressed receptor molecule. A nonlimiting example of a soluble ligand that is (indirectly) internalized into a cell via its interaction with an internalizing cell surface-expressed receptor molecule is transferrin. In embodiments, wherein the IE is transferrin (or another indirectly internalized protein), the binding of the delivery domain to the IE, and the interaction of IE with transferrin receptor (or another internalizing cell-surface expressed receptor molecule), causes the entire delivery domain, and any molecules associated therewith (e.g., the enzyme), to become internalized into the cell concurrent with the internalization of the IE and its binding partner.

In those embodiments in which the IE is indirectly internalized into a cell, the delivery domain can be, e.g., an antibody, antigen-binding fragment of an antibody, or an scFv that specifically binds IE, or a receptor or portion of a receptor that specifically interacts with the soluble effector protein. For example, if the IE is a cytokine, the delivery domain can comprise or consist of the corresponding cytokine receptor or ligand-binding portion thereof.

As used herein, “immunological reaction” generally means a patient's immunological response to an outside or “non-self’ protein. This immunological response includes an allergic reaction and the development of antibodies that interfere with the effectiveness of the replacement enzyme. Some patients may not produce any of the nonfunctioning protein, thus rendering the replacement enzyme a “foreign” protein. For example, repeated injection of recombinant GLA (rGLA) to those Fabry patients who lack GLA frequently results in an allergic reaction. In other patients, the production of antibodies against rGLA has been shown to decrease the effectiveness of the replacement enzyme in treating the disease. See for example Tajima et al. (“Use of a Modified α-N-Acetylgalactosaminidase (NAGA) in the Development of Enzyme Replacement Therapy for Fabry Disease,” 85(5) Am. J. Hum. Genet. 569-580 (2009)), which reference is incorporated herein in its entirety by reference, which discusses the use of modified NAGA as the “isozyme” to replace GLA. The modified NAGA has no immunological cross-reactivity with GLA, and “did not react to serum from a patient with Fabry disease recurrently treated with a recombinant GLA.” Id, abstract.

An “immunosuppressive agent” includes drugs and/or proteins that result in general immunosuppression and may be used to prevent cross-reactive immunological materials (CRIM) against replacement enzymes, e.g., GAA or GLA respectively in a patient with Pompe or Fabry disease. Nonlimiting examples of an immunosuppressive agent include methotrexate, mycophenolate mofetil, cyclophosphamide, rapamycin DNA alkylating agents, anti-CD20 antibody, anti-BAFF antibody, anti-CD3 antibody, anti-CD4 antibody, and any combination thereof.

Regulatory elements, e.g., promoters, that are specific to a tissue, e.g., liver, enhance expression of nucleic acid sequences, e.g., genes, under the control of such regulatory element in the tissue for which the regulatory element is specific. Nonlimiting examples of a liver specific regulatory element, e.g., liver specific promoters, may be found in Chuah et al. (2014) Mol. Ther. 22:1605-13, which reference is incorporated herein in its entirety by reference.

The term “protein” means any amino acid polymer having more than about 20 amino acids covalently linked via amide bonds. Proteins contain one or more amino acid polymer chains, generally known in the art as “polypeptides.” Thus, a polypeptide may be a protein, and a protein may contain multiple polypeptides to form a single functioning biomolecule. Disulfide bridges (i.e., between cysteine residues to form cystine) may be present in some proteins. These covalent links may be within a single polypeptide chain, or between two individual polypeptide chains. For example, disulfide bridges are essential to proper structure and function of insulin, immunoglobulins, protamine, and the like.

As used herein, “protein” includes biotherapeutic proteins, recombinant proteins used in research or therapy, trap proteins and other Fc-fusion proteins, chimeric proteins, antibodies, monoclonal antibodies, human antibodies, bispecific antibodies, antibody fragments, nanobodies, recombinant antibody chimeras, scFv fusion proteins, cytokines, chemokines, peptide hormones, and the like. Proteins may be produced using recombinant cell-based production systems, such as the insect bacculovirus system, yeast systems (e.g., Pichia sp.), mammalian systems (e.g., CHO cells and CHO derivatives like CHO-K1 cells). For a recent review discussing biotherapeutic proteins and their production, see Ghaderi et al., “Production platforms for biotherapeutic glycoproteins. Occurrence, impact, and challenges of non-human sialylation,” 28 Biotechnol Genet Eng Rev. 147-75 (2012), which reference is incorporated herein in its entirety by reference.

The term “antibody,” as used herein, includes immunoglobulin molecules comprising four polypeptide chains, two heavy (H) chains and two light (L) chains interconnected by disulfide bonds. Each heavy chain comprises a heavy chain variable region (abbreviated herein as HCVR or VH) and a heavy chain constant region. The heavy chain constant region comprises three domains, CH1, CH2 and CH3. Each light chain comprises a light chain variable region (abbreviated herein as LCVR or VL) and a light chain constant region. The light chain constant region comprises one domain, CL. The VH and VL regions can be further subdivided into regions of hypervariability, termed complementarity determining regions (CDR), interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, FR4 (heavy chain CDRs may be abbreviated as HCDR1, HCDR2 and HCDR3; light chain CDRs may be abbreviated as LCDR1, LCDR2 and LCDR3). The term “high affinity” antibody refers to those antibodies having a binding affinity to their target of at least 10⁻⁹ M, at least 10⁻¹⁰ M; at least 10⁻¹¹ M; or at least 10⁻¹² M, as measured by surface plasmon resonance, e.g., BIACORE™ or solution-affinity ELISA. The term “antibody” may encompass any type of antibody, such as monoclonal or polyclonal. Moreover, the antibody may be or any origin, such as mammalian or nonmammalian. In one embodiment, the antibody may be mammalian or avian. In a further embodiment, the antibody may be of human origin and may further be a human monoclonal antibody.

The phrase “bispecific antibody” includes an antibody capable of selectively binding two or more epitopes. Bispecific antibodies generally comprise two different heavy chains, with each heavy chain specifically binding a different epitope-either on two different molecules (e.g., antigens) or on the same molecule (e.g., on the same antigen). If a bispecific antibody is capable of selectively binding two different epitopes (a first epitope and a second epitope), the affinity of the first heavy chain for the first epitope will generally be at least one, two, three, or four orders of magnitude lower than the affinity of the first heavy chain for the second epitope, and vice versa. The epitopes recognized by the bispecific antibody can be on the same or a different target (e.g., on the same or a different protein). Bispecific antibodies can be made, for example, by combining heavy chains that recognize different epitopes of the same antigen. For example, nucleic acid sequences encoding heavy chain variable sequences that recognize different epitopes of the same antigen can be fused to nucleic acid sequences encoding different heavy chain constant regions, and such sequences can be expressed in a cell that expresses an immunoglobulin light chain. A typical bispecific antibody has two heavy chains each having three heavy chain CDRs, followed by (N-terminal to C-terminal) a CH1 domain, a hinge, a CH2 domain, and a CH3 domain, and an immunoglobulin light chain that either does not confer antigen-binding specificity but that can associate with each heavy chain, or that can associate with each heavy chain and that can bind one or more of the epitopes bound by the heavy chain antigen-binding regions, or that can associate with each heavy chain and enable binding or one or both of the heavy chains to one or both epitopes.

The phrase “heavy chain” or “immunoglobulin heavy chain” includes an immunoglobulin heavy chain constant region sequence from any organism, and unless otherwise specified includes a heavy chain variable domain. Heavy chain variable domains include three heavy chain CDRs and four FR regions, unless otherwise specified. Fragments of heavy chains include CDRs, CDRs and FRs, and combinations thereof. A typical heavy chain has, following the variable domain (from N-terminal to C-terminal), a CH1 domain, a hinge, a CH2 domain, and a CH3 domain. A functional fragment of a heavy chain includes a fragment that is capable of specifically recognizing an antigen (e.g., recognizing the antigen with a KD in the micromolar, nanomolar, or picomolar range), that is capable of expressing and secreting from a cell, and that comprises at least one CDR.

The phrase “light chain” includes an immunoglobulin light chain constant region sequence from any organism, and unless otherwise specified includes human kappa and lambda light chains. Light chain variable (VL) domains typically include three light chain CDRs and four framework (FR) regions, unless otherwise specified. Generally, a full-length light chain includes, from amino terminus to carboxyl terminus, a VL domain that includes FR1-CDR1-FR2-CDR2-FR3-CDR3-FR4, and a light chain constant domain. Light chains that can be used with this invention include e.g., those that do not selectively bind either the first or second antigen selectively bound by the antigen-binding protein. Suitable light chains include those that can be identified by screening for the most commonly employed light chains in existing antibody libraries (wet libraries or in silico), where the light chains do not substantially interfere with the affinity and/or selectivity of the antigen-binding domains of the antigen-binding proteins. Suitable light chains include those that can bind one or both epitopes that are bound by the antigen-binding regions of the antigen-binding protein.

The phrase “variable domain” includes an amino acid sequence of an immunoglobulin light or heavy chain (modified as desired) that comprises the following amino acid regions, in sequence from N-terminal to C-terminal (unless otherwise indicated): FR1, CDR1, FR2, CDR2, FR3, CDR3, FR4. A “variable domain” includes an amino acid sequence capable of folding into a canonical domain (VH or VL) having a dual beta sheet structure wherein the beta sheets are connected by a disulfide bond between a residue of a first beta sheet and a second beta sheet.

The phrase “complementarity determining region” or the term “CDR” includes an amino acid sequence encoded by a nucleic acid sequence of an organism's immunoglobulin genes that normally (i.e., in a wildtype animal) appears between two framework regions in a variable region of a light or a heavy chain of an immunoglobulin molecule (e.g., an antibody or a T cell receptor). A CDR can be encoded by, for example, a germline sequence or a rearranged or unrearranged sequence, and, for example, by a naive or a mature B cell or a T cell. In some circumstances (e.g., for a CDR3), CDRs can be encoded by two or more sequences (e.g., germline sequences) that are not contiguous (e.g., in an unrearranged nucleic acid sequence) but are contiguous in a B cell nucleic acid sequence, e.g., as the result of splicing or connecting the sequences (e.g., V-D-J recombination to form a heavy chain CDR3).

The term “antibody fragment” refers to one or more fragments of an antibody that retain the ability to specifically bind to an antigen. Examples of binding fragments encompassed within the term “antibody fragment” include (i) a Fab fragment, a monovalent fragment consisting of the VL, VH, CL and CH1 domains; (ii) a F(ab′)2 fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a dAb fragment (Ward et al. (1989) Nature 241:544-546, which reference is incorporated herein in its entirety by reference), which consists of a VH domain, (vi) an isolated CDR, and (vii) an scFv, which consists of the two domains of the Fv fragment, VL and VH, joined by a synthetic linker to form a single protein chain in which the VL and VH regions pair to form monovalent molecules. Other forms of single chain antibodies, such as diabodies are also encompassed under the term “antibody” (see e.g., Holliger et al. (1993) PNAS USA 90:6444-6448; Poljak et al. (1994) Structure 2:1121-1123, each of which reference is incorporated herein in its entirety by reference).

The phrase “Fc-containing protein” includes antibodies, bispecific antibodies, immunoadhesins, and other binding proteins that comprise at least a functional portion of an immunoglobulin CH2 and CH3 region. A “functional portion” refers to a CH2 and CH3 region that can bind a Fc receptor (e.g., an FcyR; or an FcRn, i.e., a neonatal Fc receptor), and/or that can participate in the activation of complement. If the CH2 and CH3 region contains deletions, substitutions, and/or insertions or other modifications that render it unable to bind any Fc receptor and also unable to activate complement, the CH2 and CH3 region is not functional.

Fc-containing proteins can comprise modifications in immunoglobulin domains, including where the modifications affect one or more effector function of the binding protein (e.g., modifications that affect FcyR binding, FcRn binding and thus half-life, and/or CDC activity). Such modifications include, but are not limited to, the following modifications and combinations thereof, with reference to EU numbering of an immunoglobulin constant region: 238, 239, 248, 249, 250, 252, 254, 255, 256, 258, 265, 267, 268, 269, 270, 272, 276, 278, 280, 283, 285, 286, 289, 290, 292, 293, 294, 295, 296, 297, 298, 301, 303, 305, 307, 308, 309, 311, 312, 315, 318, 320, 322, 324, 326, 327, 328, 329, 330, 331, 332, 333, 334, 335, 337, 338, 339, 340, 342, 344, 356, 358, 359, 360, 361, 362, 373, 375, 376, 378, 380, 382, 383, 384, 386, 388, 389, 398, 414, 416, 419, 428, 430, 433, 434, 435, 437, 438, and 439.

For example, and not by way of limitation, the binding protein is an Fc-containing protein and exhibits enhanced serum half-life (as compared with the same Fc-containing protein without the recited modification(s)) and have a modification at position 250 (e.g., E or Q); 250 and 428 (e.g., L or F); 252 (e.g., L/Y/F/W or T), 254 (e.g., S or T), and 256 (e.g., S/R/Q/E/D or T); or a modification at 428 and/or 433 (e.g., L/R/SI/P/Q or K) and/or 434 (e.g., H/F or Y); or a modification at 250 and/or 428; or a modification at 307 or 308 (e.g., 308F, V308F), and 434. In another example, the modification can comprise a 428L (e.g., M428L) and 434S (e.g., N434S) modification; a 428L, 2591 (e.g., V259I), and a 308F (e.g., V308F) modification; a 433K (e.g., H433K) and a 434 (e.g., 434Y) modification; a 252, 254, and 256 (e.g., 252Y, 254T, and 256E) modification; a 250Q and 428L modification (e.g., T250Q and M428L); a 307 and/or 308 modification (e.g., 308F or 308P).

The term “antigen-binding protein,” as used herein, refers to a polypeptide or protein (one or more polypeptides complexed in a functional unit) that specifically recognizes an epitope on an antigen, such as a cell-specific antigen and/or a target antigen of the present invention. An antigen-binding protein may be multi-specific. The term “multi-specific” with reference to an antigen-binding protein means that the protein recognizes different epitopes, either on the same antigen or on different antigens. A multi-specific antigen-binding protein of the present invention can be a single multifunctional polypeptide, or it can be a multimeric complex of two or more polypeptides that are covalently or noncovalently associated with one another. The term “antigen-binding protein” includes antibodies or fragments thereof of the present invention that may be linked to or co-expressed with another functional molecule, e.g., another peptide or protein. For example, an antibody or fragment thereof can be functionally linked (e.g., by chemical coupling, genetic fusion, noncovalent association or otherwise) to one or more other molecular entities, such as a protein or fragment thereof to produce a bispecific or a multi-specific antigen-binding molecule with a second binding specificity. The term “anti-” and “a” may be used interchangeable and refers to an antigen binding protein that binds a target. As a non-limiting example, “anti-TFRC,” “αTFRC,” and the like refers to an antigen binding protein that binds TfR.

As used herein, the term “epitope” refers to the portion of the antigen that is recognized by the multi-specific antigen-binding polypeptide. A single antigen (such as an antigenic polypeptide) may have more than one epitope. Epitopes may be defined as structural or functional. Functional epitopes are generally a subset of structural epitopes and are defined as those residues that directly contribute to the affinity of the interaction between the antigen-binding polypeptide and the antigen. Epitopes may also be conformational, that is, composed of nonlinear amino acids. In certain embodiments, epitopes may include determinants that are chemically active surface groupings of molecules such as amino acids, sugar side chains, phosphoryl groups, or sulfonyl groups, and, in certain embodiments, may have specific three-dimensional structural characteristics, and/or specific charge characteristics. Epitopes formed from contiguous amino acids are typically retained on exposure to denaturing solvents, whereas epitopes formed by tertiary folding are typically lost on treatment with denaturing solvents.

The term “domain” refers to any part of a protein or polypeptide having a particular function or structure. Preferably, domains of the present invention bind to cell-specific or target antigens. Cell-specific antigen- or target antigen-binding domains, and the like, as used herein, include any naturally occurring, enzymatically obtainable, synthetic, or genetically engineered polypeptide or glycoprotein that specifically binds an antigen.

The term “half-body” or “half-antibody”, which are used interchangeably, refers to half of an antibody, which essentially contains one heavy chain and one light chain. Antibody heavy chains can form dimers, thus the heavy chain of one half-body can associate with heavy chain associated with a different molecule (e.g., another half-body) or another Fc-containing polypeptide. Two slightly different Fc-domains may “heterodimerize” as in the formation of bispecific antibodies or other heterodimers, -trimers, -tetramers, and the like. See Vincent and Murini, “Current strategies in antibody engineering: Fc engineering and pH-dependent antigen binding, bispecific antibodies and antibody drug conjugates,” 7 Biotechnol. J. 1444-1450 (20912); and Shimamoto et al., “Peptibodies: A flexible alternative format to antibodies,” 4(5) MAbs 586-91 (2012), each of which references is incorporated herein in its entirety by reference.

In one embodiment, the half-body variable domain specifically recognizes the internalizing effector, and the half body Fc-domain dimerizes with an Fc-fusion protein that comprises a replacement enzyme (e.g., a peptibody) Id, 586.

The term “single-chain variable fragment” or “scFv” includes a single chain fusion polypeptide containing an immunoglobulin heavy chain variable region (VH) and an immunoglobulin light chain variable region (VL). In some embodiments, the VH and VL are connected by a linker sequence of 10 to 25 amino acids. ScFv polypeptides may also include other amino acid sequences, such as CL or CH1 regions. ScFv molecules can be manufactured by phage display or made by directly subcloning the heavy and light chains from a hybridoma or B-cell. Ahmad et al., Clinical and Developmental Immunology, volume 2012, article ID 98025 is incorporated herein by reference for methods of making scFv fragments by phage display and antibody domain cloning.

“Alpha-glucosidase” (or “α-glucosidase”), “α-glucosidase activity,” “GAA,” and “GAA activity” are used interchangeably and refer to any protein that facilitates the hydrolysis of 1,4-alpha bonds of glycogen and starch into glucose. GAA is also known inter alia as EC 3.2.1.20, maltase, glucoinvertase, glucosidosucrase, maltase-glucoamylase, alpha-glucopyranosidase, glucosidoinvertase, alpha-D-glucosidase, alpha-glucoside hydrolase, alpha-1,4-glucosidase, and alpha-D-glucoside glucohydrolase. GAA can be found in the lysosome and at the brush border of the small intestine. Patients who suffer from Pompe disease lack functioning lysosomal α-glucosidase. See S. Chiba, “Molecular mechanism in alpha-glucosidase and glucoamylase,” 61(8) Biosci. Biotechnol. Biochem. 1233-9 (1997); and Hesselink et al., “Lysosomal dysfunction in muscle with special reference to glycogen storage disease type II,” 1637(2) Biochim. Biophys. Acta. 164-70 (2003), each of which reference is incorporated herein in its entirety by reference.

“Alpha-galactosidase A” (or “α-galactosidase A”), “α-galactosidase A activity”, “α-galactosidase”, “α-galactosidase activity”, “GLA”, and “GLA activity” are used interchangeably and refer to any protein that facilitates the hydrolysis of terminal α-galactosyl moieties from glycolipids and glycoproteins, and also hydrolyses α-D-fucosides. GLA is also known inter alia as EC 3.2.1.22, melibiase, α-D-galactosidase, α-galactosidase A, α-galactoside galactohydrolase, α-D-galactoside galactohydrolase. GLA is a lysosomal enzyme encoded by the X-linked GLA gene Defects in GLA can lead to Fabry disease, in which the glycolipid known as globotriaosylceramide (a.k.a. Gb3, GL-3, or ceramide trihexoside) accumulates within blood vessels (i.e., prominent vasculopathy), resulting in pain and impairment in the function of kidney, heart, skin, and/or cerebrovascular tissues, and other tissues, and organs. See for example Prabakaran et al. “Mannose 6-phosphate receptor and sortilin mediated endocytosis of α-galactosidase A in kidney endothelial cells,” 7(6) PLoS One e39975 pp. 1-9 (2012), which reference is incorporated herein in its entirety by reference.

In one aspect, the invention provides a method of treating a patient (or subject) suffering from a lysosomal storage disease by administering to the patient a “multidomain therapeutic protein.” The multidomain therapeutic protein enters the cells of the patient and delivers to the lysosomes an enzyme or enzymatic activity (i.e., “replacement enzyme”) that replaces the enzyme or enzymatic activity (i.e., “endogenous enzyme”) that is associated with the LSD. In one embodiment, the multidomain therapeutic protein is delivered to the patient via a gene therapy vector that contains a polynucleotide that encodes the multidomain therapeutic protein.

LSDs include sphingolipidoses, a mucopolysaccharidoses, and glycogen storage diseases. In some embodiments, the LSD is any one or more of Fabry disease, Gaucher's disease type I, Gaucher's disease type II, Gaucher's disease type III, Niemann-Pick disease type A, Niemann-Pick disease type B, GM1-gangliosidosis, Sandhoff disease, Tay-Sachs disease, GM2-activator deficiency, GM3-gangliosidosis, metachromatic leukodystrophy, sphingolipid-activator deficiency, Scheie disease, Hurler-Scheie disease, Hurler disease, Hunter disease, Sanfilippo A, Sanfilippo B, Sanfilippo C, Sanfilippo D, Morquio syndrome A, Morquio syndrome B, Maroteaux-Lamy disease, Sly disease, MPS IX, and Pompe disease. In a specific embodiment, the LSD is Fabry disease. In another specific embodiment, the LSD is Pompe disease.

In some embodiments, the multidomain therapeutic protein comprises (a) the replacement enzyme and (b) a molecular entity that binds an internalizing effector (delivery domain). In some cases, the replacement enzyme is any one or more of α-galactosidase, p-galactosidase, α-glucosidase, β-glucosidase, saposin-C activator, ceramidase, sphingomyelinase, β-hexosaminidase, GM2 activator, GM3 synthase, arylsulfatase, sphingolipid activator, α-iduronidase, iduronidase-2-sulfatase, heparin N-sulfatase, N-acetyl-α-glucosaminidase, α-glucosamide N-acetyltransferase, N-acetylglucosamine-6-sulfatase, N-acetylgalactosamine-6-sulfate sulfatase, N-acetylgalactosamine-4-sulfatase, β-glucuronidase, and hyaluronidase.

In some cases, the patient may not make sufficient protein such that a replacement enzyme is recognized by the patient as “non-self” and an immunological reaction ensues after administering a replacement enzyme; this is not desirable. Therefore, in some embodiments, the replacement enzyme is designed or produced in such a way as to avoid inducing an immunological reaction in the subject. One such solution is to use an “isozyme” as a replacement enzyme. An isozyme is sufficiently close to a “self” protein of the patient but has the replacement enzyme activity sufficient to ameliorate the symptoms of the LSD.

In one particular embodiment, in which the LSD is Pompe disease and the endogenous enzyme is α-glucosidase (GAA), the isozyme can be any one of acid α-glucosidase, sucrase-isomaltase (SI), maltase-glucoamylase (MGAM), glucosidase II (GANAB), and neutral α-glucosidase (C GNAC). In another particular embodiment, in which the LSD is Fabry disease and the endogenous enzyme is α-galactosidase A (GLA), the isozyme can be an α-N-acetylgalactosaminidase engineered to have GLA activity.

Provided herein are methods, other than to use an isozyme, to reduce cross-reactive immunological materials (CRIM) against the replacement enzyme. Administration of a multidomain therapeutic protein (e.g., via a gene therapy vector) comprising an internalizing effector binding domain and the enzyme domain reduces the level of CRIM against the replacement enzyme comprised to administration of a control therapeutic protein (lacking the internalizing effector domain and comprising an enzyme domain). As such, one embodiment of reducing CRIM against an enzyme in a patient with a deficiency in the enzyme comprises administering to the patient a multidomain therapeutic protein (or nucleic acid encoding same, e.g., a gene therapy vector containing a gene encoding the multidomain therapeutic protein), wherein the multidomain therapeutic protein comprises a delivery domain (e.g., internalizing effector binding protein) and an enzyme domain.

The multidomain therapeutic protein has an internalizing effector binding protein component that enables the uptake of the replacement enzyme into the cell. Thus, in some embodiments, the internalizing effector can be transferrin receptor (TfR), CD63, MHC-I, Kremen-1, Kremen-2, LRP5, LRP6, LRP8, LDL-receptor, LDL-related protein 1 receptor, ASGR1, ASGR2, amyloid precursor protein-like protein-2 (APLP2), apelin receptor (APLNR), PRLR (prolactin receptor), MAL (myelin and lymphocyte protein, a.k.a. VIP17), IGF2R, vacuolar-type H+ ATPase, diphtheria toxin receptor, folate receptor, glutamate receptors, glutathione receptor, leptin receptor, scavenger receptor, SCARA1-5, SCARB1-3, and CD36.

In some embodiments, the internalizing effector-binding protein comprises an antigen-binding protein that includes, for example, a receptor-fusion molecule, a trap molecule, a receptor-Fc fusion molecule, an antibody, an Fab fragment, an F(ab′)2 fragment, an Fd fragment, an Fv fragment, a single-chain Fv (scFv) molecule, a dAb fragment, an isolated complementarity determining region (CDR), a CDR3 peptide, a constrained FR3-CDR3-FR4 peptide, a domain-specific antibody, a single domain antibody, a domain-deleted antibody, a chimeric antibody, a CDR-grafted antibody, a diabody, a triabody, a tetrabody, a minibody, a nanobody, a monovalent nanobody, a bivalent nanobody, a small modular immunopharmaceutical (SMIP), a camelid antibody (VHH heavy chain homodimeric antibody), and a shark variable IgNAR domain.

In one embodiment, the molecular entity that binds the internalizing effector is an antibody, an antibody fragment, or other antigen-binding protein. For example, the molecular entity can be a bispecific antibody, in which one arm binds the internalizing effector (e.g., TfR), and the other arm binds the replacement enzyme. In a specific embodiment, the disease treated is Fabry disease, and the multidomain therapeutic protein comprises GLA and a bispecific antibody that binds GLA and TfR. In another specific embodiment, the disease treated is Pompe disease, and the multidomain therapeutic protein comprises GAA and a bispecific antibody that binds GAA and TfR.

In another embodiment, the molecular entity that binds the internalizing effector comprises a half-antibody, and the replacement enzyme contains an Fc domain (enzyme-Fc fusion polypeptide). In one embodiment, the Fc domain of the enzyme-Fc fusion polypeptide associates with the Fc domain of the internalizing effector-specific half-body to form the multidomain therapeutic protein (FIG. 1).

In other embodiments, the replacement enzyme is covalently linked to an internalizing effector-binding protein. The enzyme-Fc fusion:half-body embodiment described in the previous paragraph (see also FIG. 1B) falls into this class, since the Fc dimer can be secured via one or more disulfide bridges. The covalent linkage between the enzyme activity domain or polypeptide and the internalization-binding domain or polypeptide may be any type of covalent bond, i.e., any bond that involved sharing of electrons. In some cases, the covalent bond is a peptide bond between two amino acids, such that the replacement enzyme and the internalizing effector-binding protein in whole or in part form a continuous polypeptide chain, as in a fusion protein. In some cases, the replacement enzyme portion and the internalizing effector-binding protein are directly linked. In other cases, a linker is used to tether the two portions. See Chen et al., “Fusion protein linkers: property, design and functionality,” 65(10) Adv Drug Deliv Rev. 1357-69 (2013).

The term “linker” or “spacer” refers to a short (e.g., 2 to 25 amino acids) polypeptide that typically allow for proper folding of one or more linked components of the fusion protein, e.g., a VH linked to a VL of an scFv, a therapeutic protein (e.g., replacement enzyme) linked to a delivery domain (e.g., an anti-internalizing effector antibody) of a multidomain therapeutic protein as described herein. The linker provides a flexible junction region of the component of the fusion protein, allowing the two ends of the molecule to move independently, and may play an important role in retaining each of the two moieties' appropriate functions. Therefore, the junction region acts in some cases as both a linker, which combines the two parts together, and as a spacer, which allows each of the two parts to form its own biological structure and not interfere with the other part. Furthermore, the junction region should create an epitope that will not be recognized by the subject's immune system as foreign, in other words, will not be considered immunogenic. Linker selection may also have an effect on binding activity of the fusion molecule (see Huston et al, 1988, PNAS, 85:16:5879-83; Robinson & Bates, 1998, PNAS 95(11):5929-34; Arai, et al. 2001, PEDS, 14(8):529-32; and Chen, X. et al., 2013, Advanced Drug Delivery Reviews 65:1357-1369). In one embodiment, the delivery domain is connected to the therapeutic polypeptide, or fragment thereof, via one or more peptide linkers. In another embodiment, the variable regions of an scFv antibody are connected to each other, or a fragment thereof, via one or more peptide linkers.

The length of the linker chain may be 2, 3, 4, 5, 6, 7, 8, 9, 10, 11, 12, 13, 14 15 or more amino acid residues, but typically is between 5 and 25 residues. Examples of linkers include polyGlycine linkers, such as Gly-Gly, Gly-Gly-Gly (3Gly), 4Gly, 5Gly, 6Gly, 7Gly, 8Gly or 9Gly. Examples of linkers also include Gly-Ser peptide linkers such as Ser-Gly, Gly-Ser, Gly-Gly-Ser, Ser-Gly-Gly, Gly-Gly-Gly-Ser, Ser-Gly-Gly-Gly, Gly-Gly-Gly-Gly-Ser, Ser-Gly-Gly-Gly-Gly, Gly-Gly-Gly-Gly-Gly-Ser, Ser-Gly-Gly-Gly-Gly-Gly, Gly-Gly-Gly-Gly-Gly-Gly-Ser, Ser-Gly-Gly-Gly-Gly-Gly-Gly, (Gly-Gly-Gly-Gly-Ser)ₙ, and (Ser-Gly-Gly-Gly-Gly)ₙ, wherein n=1 to 10. (Gly-Gly-Gly-Gly-Ser)ₙ and (Ser-Gly-Gly-Gly-Gly)ₙ are also known as (G4S)ₙ and (S4G)ₙ, respectively.

In some embodiments, the therapeutic protein, e.g., replacement enzyme, is covalently linked to the C-terminus of the heavy chain of an anti-internalizing effector antibody (FIG. 1C) or to the C-terminus of the light chain (FIG. 1E). In some embodiments, the replacement enzyme is covalently linked to the N-terminus of the heavy chain of an anti-internalizing effector antibody (FIG. 1D) or to the N-terminus of the light chain (FIG. 1F). In some embodiments, the enzyme is linked to the C-terminus of an anti-internalizing effector scFv domain (FIG. 1G).

In some cases, especially where the therapeutic protein, e.g., replacement enzyme, is not normally proteolytically processed in the lysosome, a cleavable linker is added to those embodiments of the multidomain therapeutic protein that comprise an antibody-enzyme fusion. In some embodiments, a cathepsin cleavable linker is inserted between the antibody and the replacement enzyme to facilitate removal of the antibody in the lysosome in order to (a) possibly help preserve enzymatic activity by removing the sterically large antibody and (b) possibly increase lysosomal half-life of the enzyme.

In one particular embodiment, the multidomain therapeutic protein is delivered to the patient or cell in a gene therapy vector that contains a polynucleotide that encodes the multidomain therapeutic protein. In one embodiment, the multidomain therapeutic protein comprises a delivery domain and an enzyme domain. In a specific embodiment, the delivery domain binds to an internalizing effector, such as TfR, CD63, MHC-I, Kremen-1, Kremen-2, LRP5, LRP6, LRP8, LDL-receptor, LDL-related protein 1 receptor, ASGR1, ASGR2, amyloid precursor protein-like protein-2 (APLP2), apelin receptor (APLNR), MAL (myelin and lymphocyte protein), IGF2R, vacuolar-type H+ ATPase, diphtheria toxin receptor, folate receptor, glutamate receptors, glutathione receptor, leptin receptors, scavenger receptor A1-5 (SCARA1-5), SCARB1-3, or CD36. In one embodiment, the delivery domain is a single-chain variable fragment (scFv) that binds to CD63 (i.e., anti-CD63 scFv). In another embodiment, the delivery domain is a single-chain variable fragment (scFv) that binds to TfRC (i.e., anti-TfRC scFv).

In one particular embodiment, the enzyme domain of the multidomain therapeutic protein comprises a hydrolase. In a specific embodiment, the enzyme domain comprises a hydrolase that is a glycosylase. In a more specific embodiment, the enzyme domain comprises a glycosylase that is a glycosidase. In a more specific embodiment, the enzyme domain is a glycosidase that is alpha-glucosidase.

Generally, disclosed herein are compositions comprising and use of polynucleotides, e.g., (m)RNA, DNA, and modified forms thereof, that encode a multidomain therapeutic protein comprising an internalizing effector domain and an enzyme domain in the treatment of lysosomal storage diseases, e.g., for the reduction of glycogen and/or the enhancement of immune tolerance for GAA in a patient with Pompe disease.

The term “polynucleotide” includes a polymer of nucleotides (e.g., RNA or DNA) that encodes at least one polypeptide, including fusion polypeptides, e.g., a multidomain therapeutic polypeptide comprising an internalizing effector domain and an enzyme domain. Polynucleotide as used herein encompasses polymers comprising both modified and unmodified nucleotides. A polynucleotide may contain one or more coding and noncoding regions. A polynucleotide can be purified from natural sources, produced using recombinant expression systems and optionally purified, chemically synthesized, etc. Where appropriate, e.g., in the case of chemically synthesized molecules, a polynucleotide can comprise nucleoside analogs such as analogs having chemically modified bases or sugars, backbone modifications, etc. A polynucleotide sequence is presented in the 5′ to 3′ direction unless otherwise indicated. In some embodiments, a polynucleotide is or comprises natural nucleosides (e.g., adenosine, guanosine, cytidine, uridine); nucleoside analogs (e.g., 2-aminoadenosine, 2-thiothymidine, inosine, pyrrolo-pyrimidine, 3-methyl adenosine, 5-methylcytidine, C-5 propynyl-cytidine, C-5 propynyl-uridine, 2-aminoadenosine, C5-bromouridine, C5-fluorouridine, C5-iodouridine, C5-propynyl-uridine, C5-propynyl-cytidine, C5-methylcytidine, 2-aminoadenosine, 7-deazaadenosine, 7-deazaguanosine, 8-oxoadenosine, 8-oxoguanosine, 0(6)-methylguanine, and 2-thiocytidine); chemically modified bases; biologically modified bases (e.g., methylated bases); intercalated bases; modified sugars (e.g., 2′-fluororibose, ribose, 2′-deoxyribose, arabinose, and hexose); and/or modified phosphate groups (e.g., phosphorothioates and 5′-N-phosphoramidite linkages).

In some embodiments, a polynucleotide comprises one or more nonstandard nucleotide residues. The nonstandard nucleotide residues may include, e.g., 5-methyl-cytidine (“5mC”), pseudouridine (“WU”), and/or 2-thio-uridine (“2sU”). See, e.g., U.S. Pat. No. 8,278,036 or WO2011012316, each of which is incorporated in its entirety by reference for a discussion of such residues and their incorporation into a polynucleotide. The presence of nonstandard nucleotide residues may render a polynucleotide more stable and/or less immunogenic than a polynucleotide with the same sequence but containing only standard residues. In further embodiments, a polynucleotide may comprise one or more nonstandard nucleotide residues chosen from isocytosine, pseudoisocytosine, 5-bromouracil, 5-propynyluracil, 6-aminopurine, 2-aminopurine, inosine, diaminopurine and 2-chloro-6-aminopurine cytosine, as well as combinations of these modifications and other nucleobase modifications. Certain embodiments may further include additional modifications to the furanose ring or nucleobase. Additional modifications may include, for example, sugar modifications or substitutions (e.g., one or more of a 2′-O-alkyl modification, a locked nucleic acid (LNA)). In some embodiments, the polynucleotide may be complexed or hybridized with additional polynucleotides and/or peptide polynucleotides (PNA). In embodiments where the sugar modification is a 2′-O-alkyl modification, such modifications may include, but are not limited to a 2′-deoxy-2′-fluoro modification, a 2′-O-methyl modification, a 2′-O-methoxyethyl modification, and a 2′-deoxy modification. In certain embodiments, any of these modifications may be present in 0-100% of the nucleotides—for example, more than 0%, 1%, 10%, 25%, 50%, 75%, 85%, 90%, 95%, or 100% of the constituent nucleotides individually or in combination. In some embodiments, a polynucleotide comprises messenger RNA (mRNA) molecules, which may or may not be modified, e.g., which may or may not comprise a modified nucleotide, by well-known methods to increase their stability and/or decrease their immunogenicity. In some embodiments, a polynucleotide comprises DNA molecules, which may or may not be modified, e.g., which may or may not comprise a modified nucleotide, by well-known methods to increase their stability and/or decrease their immunogenicity.

In some embodiments, the polynucleotide also includes a “locus-targeting nucleic acid sequence.” The locus targeting sequence enables the integration of the multidomain therapeutic protein-encoding polynucleotide into the genome of the recipient host cell. In some embodiments, the locus targeting sequence includes flanking homology arms to enable homologous recombination. In some embodiments, the locus targeting sequence includes guide RNA sequences and a type II Cas enzyme to facilitate integration (i.e., the CRISPR-Cas9 method). In some embodiments, the locus targeting sequence includes guide zinc-finger nuclease (ZFN) recognition sequences to facilitate integration. In some embodiments, the locus targeting sequence includes transcription activator-like effector nuclease (TALEN) recognition sequences to facilitate integration. In still other embodiments, the locus targeting sequence includes a single residue-to-nucleotide code used by BuD-derived nucleases to facilitate integration.

In some embodiments, the genomic locus into which the multidomain therapeutic protein-encoding polynucleotide is integrated is a “safe harbor locus.” In one embodiment, a “safe harbor locus” enables high expression of the multidomain therapeutic protein, while not interfering with the expression of essential genes or promoting the expression of oncogenes or other deleterious genes. In one embodiment, the genomic locus is at or proximal to the liver-expressed albumin (Alb) locus, a EESYR locus, a SARS locus, position 188,083,272 of human chromosome 1 or its nonhuman mammalian orthologue, position 3,046,320 of human chromosome 10 or its nonhuman mammalian orthologue, position 67,328,980 of human chromosome 17 or its nonhuman mammalian orthologue, an adeno-associated virus site 1 (AAVS1; a naturally occurring site of integration of AAV virus) on human chromosome 19 or its nonhuman mammalian orthologue, a chemokine receptor 5 (CCR5) gene, a chemokine receptor gene encoding an HIV-1 coreceptor, or a mouse Rosa26 locus or its nonmurine mammalian orthologue. In one embodiment, the genomic locus is an adeno-associated virus site. In one embodiment, the genomic locus for integration is selected according to the method of Papapetrou and Schambach, J. Molecular Therapy, vol. 24(4):678-684, April 2016, which is herein incorporated by reference in its entirety for the step-wise selection of a safe harbor genomic locus for gene therapy vector integration; see also Barzel et al. Nature, vol. 517:360-364, which is herein incorporated by reference in its entirety, for the promoterless gene targeting into the liver-expressed albumin (Alb) locus.

In some embodiments, the polynucleotide, e.g., DNA, also contains a promoter operably linked to the multidomain therapeutic protein-encoding nucleic acid sequence. In a specific embodiment, the promoter is a tissue-specific promotor that drives gene expression in a particular tissue. In one embodiment, the tissue specific promoter is a liver-specific enhancer/promoter derived from serpina1 (e.g., SEQ ID NO:9) and/or is a TTR promoter (SEQ ID NO:8). In other embodiments, the promoter is a CMV promoter. In other embodiments, the promoter is a ubiquitin C promoter.

In one embodiment, the multidomain therapeutic protein-encoding “gene therapy vector” is any vector capable of delivering the polynucleotide encoding the multidomain therapeutic protein to a host, e.g., a patient. In some embodiments, the gene therapy vector targets a specific host cell or organ, e.g., for local delivery, e.g., tissue-specific delivery. Typically, local delivery requires a protein (e.g., a multidomain therapeutic protein) encoded by mRNAs to be translated and expressed mainly in and/or by an organ, e.g., a liver, whereby thereby forming a depot, e.g., a liver depot for production (and secretion) of the protein. In some embodiments, a gene therapy vector delivers a multidomain therapeutic protein polynucleotide to the liver in a patient to form a liver depot. See, e.g., DeRosa et al. Gene Therapy, vol. 10:699-707, incorporated herein by reference in its entirety. In some embodiments, a gene therapy vector delivers a polynucleotide encoding a multidomain therapeutic protein to muscle tissue in a patient. In some embodiments, a gene therapy vector delivers a polynucleotide encoding a multidomain therapeutic protein to the brain of a patient.

Any now-known or future-developed gene therapy delivery vector, natural or engineered, can be used in the practice of this invention. In some embodiments, the gene therapy vector is a viral vector, e.g., comprises a virus, viral capsid, viral genome etc. In some embodiments, the gene therapy vector is a naked polynucleotide, e.g., an episome. In some embodiments, the gene therapy vector comprises a polynucleotide complex. Exemplary nonlimiting polynucleotide complexes for use as a gene therapy vector include lipoplexes, polymersomes, polypexes, dendrimers, inorganic nanoparticles (e.g., polynucleotide coated gold, silica, iron oxide, calcium phosphate, etc.). In some embodiments, a gene therapy vector as described herein comprises a combination of a viral vector, naked polynucleotides, and polynucleotide complexes.

In one embodiment, the gene therapy vector is a virus, including a retrovirus, adenovirus, herpes simplex virus, pox virus, vaccinia virus, lentivirus, or an adeno-associated virus. In one embodiment, the gene therapy vector is an adeno-associated virus (AAV), including serotypes AAV1, AAV2, AAV3, AAV4, AAV5, AAV6, AAV7, AAV8, AAV9, AAV10, and AAV11, or engineered or naturally selected variants thereof.

In one embodiment, the polynucleotide also contains adeno-associated virus (AAV) nucleic acid sequence. In one embodiment, the gene therapy vector is a chimeric adeno-associated virus containing genetic elements from two or more serotypes. For example, an AAV vector with rep genes from AAV1 and cap genes from AAV2 (designated as AAV1/2 or AAV RC1/2) may be used as a gene therapy vector to deliver the multidomain therapeutic protein polynucleotide to a cell or a cell of a patient in need. In one embodiment, the gene therapy vector is an AAV1/2, AAV1/3, AAV1/4, AAV1/5, AAV1/6, AAV1/7, AAV1/8, AAV1/9, AAV1/10, AAV1/11, AAV2/1, AAV2/3, AAV2/4, AAV2/5, AAV2/6, AAV2/7, AAV2/8, AAV2/9, AAV2/10, AAV2/11, AAV3/1, AAV3/2, AAV3/4, AAV3/5, AAV3/6, AAV3/7, AAV3/8, AAV3/9, AAV3/10, AAV3/10, AAV4/1, AAV4/2, AAV4/3, AAV4/5, AAV4/6, AAV4/7, AAV4/8, AAV4/9, AAV4/10, AAV4/11, AAV5/1, AAV5/2, AAV5/3, AAV5/4, AAV5/6, AAV5/7, AAV5/8, AAV5/9, AAV5/10, AAV5/11, AAV6/1, AAV6/2, AAV6/3, AAV6/4, AAV6/5, AAV6/7, AAV6/8, AAV6/9, AAV6/10, AAV6/10, AAV7/1, AAV7/2, AAV7/3, AAV7/4, AAV7/5, AAV7/6, AAV7/8, AAV7/9, AAV7/10, AAV7/11, AAV8/1, AAV8/2, AAV8/3, AAV8/4, AAV8/5, AAV8/6, AAV8/7, AAV8/9, AAV8/10, AAV8/11, AAV9/1, AAV9/2, AAV9/3, AAV9/4, AAV9/5, AAV9/6, AAV9/7, AAV9/8, AAV9/10, AAV9/11, AAV10/1, AAV10/2, AAV10/3, AAV10/4, AAV10/5, AAV10/6, AAV10/7, AAV10/8, AAV10/9, AAV10/11, AAV11/1, AAV11/2, AAV11/3, AAV11/4, AAV11/5, AAV11/6, AAV11/7, AAV11/8, AAV11/9, AAV11/10, chimeric viral vector or derivatives thereof. Gao et al., “Novel adeno-associated viruses from rhesus monkeys as vectors for human gene therapy,” PNAS 99(18): 11854-11859, Sep. 3, 2002, is incorporated herein by reference for AAV vectors and chimeric viral vectors useful as gene therapy vectors, and their construction and use.

In a more specific embodiment, the gene therapy vector is a chimeric AAV vector with a serotype 2 rep gene sequence and a serotype 8 cap sequence (“AAV2/8” or “AAV RC2/8).

In some embodiments, the gene therapy vector is a viral vector that has been pseudotyped (e.g., engineered) to target a specific cell, e.g., a hepatocyte. Many of the advances in targeted gene therapy using viral vectors may be summarized as nonrecombinatorial (nongenetic) or recombinatorial (genetic) modification of the viral vector, which result in the pseudotyping, expanding, and/or retargeting of the natural tropism of the viral vector (reviewed in Nicklin and Baker (2002) Curr. Gene Ther. 2:273-93; Verheiji and Rottier (2012) Advances Virol 2012:1-15; each of which references is incorporated herein in its entirety by reference). Nongenetic approaches typically utilize an adaptor, which recognizes both a wildtype (nonmodified) virus surface protein and a target cell. Soluble pseudo-receptors (for the wildtype virus), polymers such as polyethylene glycol, and antibodies or portions thereof, have been used as the virus binding domain of the adaptors, while natural peptide or vitamin ligands, and antibodies and portions thereof have been used for the cell binding domain of the adaptors described above. For example, retargeting of the viral vector to a target cell may be accomplished upon binding of the vector:adaptor complex to a protein expressed on the surface of the target cell, e.g., a cell surface protein. Such approach has been used for: AAV (Bartlett et al. (1999) Nat. Biotechnol. 74: 2777-2785); adenoviruses (Hemminki et al. (2001) Cancer Res. 61: 6377-81; van Beusechem et al. (2003) Gene Therapy 10:1982-1991; Einfeld, et al. (2001) J. Virol. 75:11284-91; Glasgow et al. (2009) PLOS One 4:e8355); herpesviruses (Nakano et al. (2005) Mol. Ther. 11:617-24); paramyxoviruses (Bian et al. (2005) Cancer Gene Ther. 12:295-303; Bian et al. (2005) Int. J. Oncol. 29:1359-69); and coronaviruses (Haijema et al. (2003) J. Virol. 77:4528-4538; Wurdinger et al. (2005) Gene Therapy 12:1394-1404); each of which references is incorporated herein in its entirety by reference.

A more popular approach has been the recombinatorial genetic modification of viral capsid proteins, and thus the surface of the viral capsid. In indirect recombinatorial approaches, a viral capsid is modified with a heterologous “scaffold,” which then links to an adaptor. The adaptor binds to the scaffold and the target cell (Arnold et al. (2006) Mol. Ther. 5:125-132; Ponnazhagen et al. (2002) J. Virol. 76:12900-907; see also WO 97/05266 each of which references is incorporated herein in its entirety by reference). Scaffolds such as (1) Fc binding molecules (e.g., Fc receptors, Protein A, etc.), which bind to the Fc of antibody adaptors, (2) (strept)avidin, which binds to biotinylated adaptors, (3) biotin, which binds to adaptors fused with (strept)avidin, and (4) protein:protein binding pairs that form isometric peptide bonds such as SpyCatcher, which binds a SpyTagged adaptor, have been incorporated into Ad (Pereboeva et al. (2007) Gene Therapy 14: 627-637; Park et al. (2008) Biochemical and Biophysical Research Communications 366: 769-774; Henning et al. (2002) Human Gene Therapy 13:1427-1439; Banerjee et al. (2011) Bioorganic and Medicinal Chemistry Letters 21:4985-4988); AAV (Gigout et al. (2005) Molecular Therapy 11:856-865; Stachler et al. (2008) Molecular Therapy 16:1467-1473); and togavirus (Quetglas et al. (2010) Virus Research 153:179-196; Ohno et al. (1997) Nature Biotechnology 15:763-767; Klimstra et al. (2005) Virology 338:9-21; each of which references is incorporated herein in its entirety by reference).

In a direct recombinatorial targeting approach, a targeting ligand is directly inserted into, or coupled to, a viral capsid, i.e., protein viral capsids are modified to express a heterologous ligand. The ligand than redirects, e.g., binds, a receptor or marker preferentially or exclusively expressed on a target cell (Stachler et al. (2006) Gene Ther. 13:926-931; White et al. (2004) Circulation 109:513-519; each of which references is incorporated herein in its entirety by reference). Direct recombinatorial approaches have been used in AAV (Park et al., (2007) Frontiers in Bioscience 13:2653-59; Girod et al. (1999) Nature Medicine 5:1052-56; Grifman et al. (2001) Molecular Therapy 3:964-75; Shi et al. (2001) Human Gene Therapy 12:1697-1711; Shi and Bartlett (2003) Molecular Therapy 7:515-525, each of which references is incorporated herein in its entirety by reference); retrovirus (Dalba et al. Current Gene Therapy 5:655-667; Tai and Kasahara (2008) Frontiers in Bioscience 13:3083-3095; Russell and Cosset (1999) Journal of Gene Medicine 1:300-311; Erlwein et al. (2002) Virology 302:333-341; Chadwick et al. (1999) Journal of Molecular Biology 285:485-494; Pizzato et al. (2001) Gene Therapy 8:1088-1096); poxvirus (Guse et al. (2011) Expert Opinion on Biological Therapy 11:595-608; Galmiche et al. (1997) Journal of General Virology 78:3019-3027; Paul et al. (2007) Viral Immunology 20:664-671); paramyxovirus (Nakamura and Russell (2004) Expert Opinion on Biological Therapy 4:1685-1692; Hammond et al. (2001) Journal of Virology 75:2087-2096; Galanis (2010) Clinical Pharmacology and Therapeutics 88:620-625; Blechacz and Russell (2008) Current Gene Therapy 8:162-175; Russell and Peng (2009) Current Topics in Microbiology and Immunology 330:213-241); and herpesvirus (Shah and Breakefield (2006) Current Gene Therapy 6:361-370; Campadelli-Fiume et al. (2011) Reviews in Medical Virology 21:213-226; each of which references is incorporated herein in its entirety by reference).

In some embodiments, a gene therapy vector as described herein is pseudotyped to those tissues that are particularly suited for generating a regulatory response, e.g., tolerance toward, e.g., the replacement enzyme. Such tissues include, but are not limited to mucosal tissue, e.g., gut-associated lymphoid tissue (GALT), hematopoietic stem cells, and the liver. In some embodiments, the gene therapy vector, or gene encoding a multidomain therapeutic protein as described herein is expressed under the control of promoters specific for those tissues, e.g., a liver-specific promoter.

In some embodiments, a gene therapy vector as described herein comprises a naked polynucleotide. For example, in some embodiments, a polynucleotide encoding a multidomain therapeutic polypeptide may be injected, e.g., intramuscularly, directly into an organ for the formation of a depot, intravenously, etc. Additional methods well-known for the enhanced delivery of naked polynucleotides include but are not limited to electroporation, sonoporation, use of a gene gun to shoot polynucleotides coated gold particles, magnetofection, and hydrodynamic delivery.

In some embodiments, a gene therapy vector as described herein comprises polynucleotide complexes, such as, but not limited to, nanoparticles (e.g., polynucleotide self-assembled nanoparticles, polymer-based self-assembled nanoparticles, inorganic nanoparticles, lipid nanoparticles, semiconductive/metallic nanoparticles), gels and hydrogels, polynucleotide complexes with cations and anions, microparticles, and any combination thereof.

In some embodiments, the polynucleotides disclosed herein may be formulated as self-assembled nanoparticles. As a nonlimiting example, polynucleotides may be used to make nanoparticles which may be used in a delivery system for the polynucleotides (see, e.g., International Pub. No. WO 2012/125987; herein incorporated by reference in its entirety). In some embodiments, the polynucleotide self-assembled nanoparticles may comprise a core of the polynucleotides disclosed herein and a polymer shell. The polymer shell may be any of the polymers described herein and are known in the art. In an additional embodiment, the polymer shell may be used to protect the polynucleotides in the core.

In some embodiments, these self-assembled nanoparticles may be microsponges formed of long polymers of polynucleotide hairpins which form into crystalline ‘pleated’ sheets before self-assembling into microsponges. These microsponges are densely packed sponge like microparticles which may function as an efficient carrier and may be able to deliver cargo to a cell. The microsponges may be from 1 μm to 300 nm in diameter. The microsponges may be complexed with other agents known in the art to form larger microsponges. As a nonlimiting example, the microsponge may be complexed with an agent to form an outer layer to promote cellular uptake such as polycation polyethyleneime (PEI). This complex can form a 250 nm-diameter particle that can remain stable at high temperatures (150° C.; Grabow and Jaegar, Nature Materials 2012, 11:269-269; herein incorporated by reference in its entirety). Additionally, these microsponges may be able to exhibit an extraordinary degree of protection from degradation by ribonucleases. In another embodiment, the polymer-based self-assembled nanoparticles such as, but not limited to, microsponges, may be fully programmable nanoparticles. The geometry, size and stoichiometry of the nanoparticle may be precisely controlled to create the optimal nanoparticle for delivery of cargo such as, but not limited to, polynucleotides.

In some embodiments, polynucleotides may be formulated in inorganic nanoparticles (U.S. Pat. No. 8,257,745, herein incorporated by reference in its entirety). The inorganic nanoparticles may include, but are not limited to, clay substances that are water swellable. As a nonlimiting example, the inorganic nanoparticle may include synthetic smectite clays that are made from simple silicates (see, e.g., U.S. Pat. Nos. 5,585,108 and 8,257,745 each of which are herein incorporated by reference in their entirety).

In some embodiments, a polynucleotide may be formulated in water-dispersible nanoparticle comprising a semiconductive or metallic material (U.S. Pub. No. 20120228565; herein incorporated by reference in its entirety) or formed in a magnetic nanoparticle (U.S. Pub. No. 20120265001 and 20120283503; each of which is herein incorporated by reference in its entirety). The water-dispersible nanoparticles may be hydrophobic nanoparticles or hydrophilic nanoparticles.

In some embodiments, the polynucleotides disclosed herein may be encapsulated into any hydrogel known in the art which may form a gel when injected into a subject. Hydrogels are a network of polymer chains that are hydrophilic and are sometimes found as a colloidal gel in which water is the dispersion medium. Hydrogels are highly absorbent (they can contain over 99% water) natural or synthetic polymers. Hydrogels also possess a degree of flexibility very similar to natural tissue, due to their significant water content. The hydrogel described herein may be used to encapsulate lipid nanoparticles which are biocompatible, biodegradable and/or porous.

As a nonlimiting example, the hydrogel may be an aptamer-functionalized hydrogel. The aptamer-functionalized hydrogel may be programmed to release one or more polynucleotides using polynucleotide hybridization. (Battig et al., J. Am. Chem. Society. 2012 134:12410-12413; herein incorporated by reference in its entirety). In some embodiments, the polynucleotide may be encapsulated in a lipid nanoparticle and then the lipid nanoparticle may be encapsulated into a hydrogel.

In some embodiments, the polynucleotides disclosed herein may be encapsulated into a fibrin gel, fibrin hydrogel, or fibrin glue. In another embodiment, the polynucleotides may be formulated in a lipid nanoparticle or a rapidly eliminated lipid nanoparticle prior to being encapsulated into a fibrin gel, fibrin hydrogel, or fibrin glue. In yet another embodiment, the polynucleotides may be formulated as a lipoplex prior to being encapsulated into a fibrin gel, fibrin hydrogel, or fibrin glue. Fibrin gels, hydrogels, and glues comprise two components, a fibrinogen solution and a thrombin solution which is rich in calcium (see, e.g., Spicer and Mikos, Journal of Controlled Release (2010) 148: 49-55; Kidd et al. Journal of Controlled Release (2012) 157:80-85; each of which is herein incorporated by reference in its entirety). The concentration of the components of the fibrin gel, hydrogel, and/or glue can be altered to change the characteristics, the network mesh size, and/or the degradation characteristics of the gel, hydrogel, and/or glue such as, but not limited to, changing the release characteristics of the fibrin gel, hydrogel, and/or glue (see, e.g., Spicer and Mikos, Journal of Controlled Release 2010. 148: 49-55; Kidd et al. Journal of Controlled Release 2012. 157:80-85; Catelas et al. Tissue Engineering 2008. 14:119-128; each of which is herein incorporated by reference in its entirety). This feature may be advantageous when used to deliver the polynucleotide disclosed herein (see, e.g., Kidd et al. Journal of Controlled Release 2012. 157:80-85; Catelas et al. Tissue Engineering 2008. 14:119-128; each of which is herein incorporated by reference in its entirety).

In some embodiments, a polynucleotide disclosed herein may include cations or anions. In one embodiment, the formulations include metal cations such as, but not limited to, Zn2+, Ca2+, Cu2+, Mg+ and combinations thereof. As a nonlimiting example, formulations may include polymers and a polynucleotide complexed with a metal cation (see, e.g., U.S. Pat. Nos. 6,265,389 and 6,555,525, each of which is herein incorporated by reference in its entirety).

In some embodiments, a polynucleotide may be formulated in nanoparticles and/or microparticles. These nanoparticles and/or microparticles may be molded into any size, shape, and chemistry. As an example, the nanoparticles and/or microparticles may be made using the PRINT® technology by LIQUIDA TECHNOLOGIES® (Morrisville, N.C.) (See, e.g., International Pub. No. WO2007024323; herein incorporated by reference in its entirety).

In some embodiments, the polynucleotides disclosed herein may be formulated in NanoJackets and NanoLiposomes by Keystone Nano (State College, Pa.). NanoJackets are made of compounds that are naturally found in the body including calcium and phosphate; they may also include a small amount of silicates. Nanojackets may range in size from 5 to 50 nm and may be used to deliver hydrophilic and hydrophobic compounds such as, but not limited to, polynucleotides, primary constructs and/or polynucleotide. NanoLiposomes are made of lipids such as, but not limited to, lipids which naturally occur in the body. NanoLiposomes may range in size from 60-80 nm and may be used to deliver hydrophilic and hydrophobic compounds such as, but not limited to, polynucleotides, primary constructs and/or polynucleotide. In one aspect, the polynucleotides disclosed herein are formulated in a NanoLiposome such as, but not limited to, Ceramide NanoLiposomes.

In one embodiment, the multidomain therapeutic protein is an anti-CD63 scFv-GAA fusion protein or an anti-TfR scFv-GAA fusion protein. The administration of the anti-CD63 scFv-GAA fusion protein or the anti-TfR scFv-GAA fusion protein via AAV-delivery provides long term stable production of GAA in the serum of the patient after administration of the multidomain therapeutic protein-harboring gene therapy vector. In one embodiment, the level of GAA in the serum of the recipient patient is ≥1.5 fold to 100 fold, ≥1.5 fold to 10 fold, ≥2.5 fold, 2.5 fold-3 fold, 2.5 fold, 2.6 fold, 2.7 fold, 2.8 fold, 2.9 fold, 3.0 fold, 3.1 fold, 3.2 fold, 3.3 fold, 3.4 fold, 3.5 fold, 3.6 fold, 3.7 fold, 3.8 fold, 3.9 fold, 4 fold, 5 fold, 6 fold, 7 fold, 8 fold, 9 fold, or 10 fold greater than the serum levels of a patient receiving GAA not linked to a delivery domain after 1 month, 3 months, 4 months, 5 months, or 6 months or longer after administration of the multidomain therapeutic protein-harboring gene therapy vector.

In one embodiment, the administration of the anti-CD63 scFv-GAA fusion protein or the anti-TfR scFv-GAA fusion protein via AAV-delivery provides long term stable reduction in stored glycogen levels in patients with Pompe disease. In one embodiment, the glycogen levels in heart, skeletal muscle, and liver tissue in the patient are reduced to wildtype (nondisease) levels. In one embodiment, the glycogen levels in heart, skeletal muscle, and liver tissue in the patient are maintained at wildtype levels 1 month, 2 months, 3 months, 4 months, 5 months, or 6 months or longer after administration of the multidomain therapeutic protein-harboring gene therapy vector.

In one embodiment, the administration of the anti-CD63 scFv-GAA fusion protein or the anti-TfR scFv-GAA fusion protein via AAV-delivery provides long term restoration of muscle strength in patients with Pompe disease. In one embodiment, the strength of the patient as measured by grip strength is restored to normal (i.e., nondisease normal levels) 1 month, 2 months, 3 months, 4 months, 5 months, or 6 months or longer after administration of the multidomain therapeutic protein-harboring gene therapy vector.

In one embodiment, the administration of the anti-TfR scFv-GAA fusion protein via AAV-delivery provides long term effects on the production of GAA and the storage of glycogen in neurons, oligodendrocytes, microglia, and astrocytes in the brain, as well as the restoration of normal brain functions in patients with Pompe disease. In one embodiment, these effects (i.e., nondisease normal levels) persist for 1 month, 2 months, 3 months, 4 months, 5 months, or 6 months or longer after administration of the multidomain therapeutic protein-harboring gene therapy vector.

In another aspect, the invention provides a composition comprising an enzyme activity and an antigen-binding protein, wherein the enzyme is associated with an enzyme-deficiency disease (LSD) and internalizing effector-binding protein. Enzymes (which include proteins that are not per se catalytic) associated with lysosomal storage diseases include for example any and all hydrolases, α-galactosidase, β-galactosidase, α-glucosidase, β-glucosidase, saposin-C activator, ceramidase, sphingomyelinase, β-hexosaminidase, GM2 activator, GM3 synthase, arylsulfatase, sphingolipid activator, α-iduronidase, iduronidase-2-sulfatase, heparin N-sulfatase, N-acetyl-α-glucosaminidase, α-glucosamide N-acetyltransferase, N-acetylglucosamine-6-sulfatase, N-acetylgalactosamine-6-sulfate sulfatase, N-acetylgalactosamine-4-sulfatase, β-glucuronidase, hyaluronidase, and the like.

Internalizing effector-binding proteins for example include a receptor-fusion molecule, a trap molecule, a receptor-Fc fusion molecule, an antibody, an Fab fragment, an F(ab′)2 fragment, an Fd fragment, an Fv fragment, a single-chain Fv (scFv) molecule, a dAb fragment, an isolated complementarity determining region (CDR), a CDR3 peptide, a constrained FR3-CDR3-FR4 peptide, a domain-specific antibody, a single domain antibody, a domain-deleted antibody, a chimeric antibody, a CDR-grafted antibody, a diabody, a triabody, a tetrabody, a minibody, a nanobody, a monovalent nanobody, a bivalent nanobody, a small modular immunopharmaceutical (SMIP), a camelid antibody (VHH heavy chain homodimeric antibody), a shark variable IgNAR domain, other antigen-binding proteins, and the like.

Internalizing effectors include for example TfR, CD63, MHC-I, Kremen-1, Kremen-2, LRP5, LRP6, LRP8, LDL-receptor, LDL-related protein 1 receptor, ASGR1, ASGR2, amyloid precursor protein-like protein-2 (APLP2), apelin receptor (APLNR), PRLR, MAL (Myelin And Lymphocyte protein, a.k.a. VIP17), IGF2R, vacuolar-type H+ ATPase, diphtheria toxin receptor, folate receptor, glutamate receptors, glutathione receptor, leptin receptor, scavenger receptor, SCARA1-5, SCARB1-3, and CD36. In certain embodiments, the internalizing effector is a kidney specific internalizer, such as CDH16 (Cadheri-16), CLDN16 (Claudn-16), KL (Klotho), PTH1R (parathyroid hormone receptor), SLC22A13 (Solute carrier family 22 member 13), SLC5A2 (Sodium/glucose cotransporter 2), and UMOD (Uromodulin). In other certain embodiments, the internalizing effector is a muscle specific internalizer, such as BMPR1A (Bone morphogenetic protein receptor 1A), m-cadherin, CD9, MuSK (muscle-specific kinase), LGR4/GPR48 (G protein-coupled receptor 48), cholinergic receptor (nicotinic) alpha 1, CDH15 (Cadheri-15), ITGA7 (Integrin alpha-7), CACNG1 (L-type calcium channel subunit gamma-1), CACNA15 (L-type calcium channel subunit alpha-15), CACNG6 (L-type calcium channel subunit gamma-6), SCN1B (Sodium channel subunit beta-1), CHRNA1 (ACh receptor subunit alpha), CHRND (ACh receptor subunit delta), LRRC14B (Leucine-rich repeat-containing protein 14B), dystroglycan (DAG1), and POPDC3 (Popeye domain-containing protein 3). In some specific embodiments, the internalizing effector is TfR, ITGA7, CD9, CD63, ALPL2, ASGR1, ASGR2 or PRLR.

In some embodiments, the enzyme is covalently linked to the antigen-binding protein. In one particular embodiment, the internalizing effector-binding protein consists of or contains a half-body; the enzyme is fused to an Fc-fusion domain (e.g., at the C-terminus); and the Fc-domain that is covalently linked to the enzyme associates with the Fc-domain of the antigen-binding protein, such that the association contains one or more disulfide bridges. This particular embodiment is schematically depicted in FIG. 1A, panel B.

In another particular embodiment, the internalizing effector-binding protein (delivery domain) consists of or contains an antibody or an antibody fragment, and the enzyme is covalently linked to the antibody or antibody fragment. In a specific embodiment, the delivery domain is an antibody, and the enzyme is covalently linked (directly through a peptide bond, or indirectly via a linker) to the C-terminus of the heavy chain or the light chain of the antibody (FIG. 1A, panels C or E, respectively). In another specific embodiment, the delivery domain is an antibody, and the enzyme is covalently linked (directly through a peptide bond, or indirectly via a linker) to the N-terminus of the heavy chain or the light chain of the antibody (FIG. 1A, panels D or F, respectively).

In some embodiments, the enzyme and delivery domain are not covalently linked, but are combined in an admixture. The delivery domain and the enzyme can associate through noncovalent forces to form a complex. For example, in one particular embodiment, the delivery domain is a bispecific antibody in which one arm of the antibody binds the internalizing effector and the other arm binds the enzyme. This embodiment is schematically depicted in FIG. 1A, panel A.

In some embodiments, the enzyme is GAA or comprises GAA activity (e.g., an isozyme with GAA activity), and the internalizing effector is TfR, ITGA7, CDH15, CD9, CD63, APLP2, ASGR1, ASGR2 or PRLR. In a particular embodiment, the enzyme is GAA or comprises GAA activity, the internalization domain is CD63, and the delivery domain is a bispecific antibody with specificity for CD63 and GAA. In a particular embodiment, the enzyme is GAA or comprises GAA activity, the internalization domain is TfR, and the delivery domain is a bispecific antibody with specificity for TfR and GAA.

In some embodiments, the enzyme is GLA or comprises GLA activity (e.g., an isozyme with GAA activity), and the internalizing effector is TfR, ITGA7, CD9, CD63, APLP2, ASGR1, ASGR2, or PRLR. In a particular embodiment, the enzyme is GLA or comprises GLA activity, the internalization domain is CD63, and the delivery domain is a bispecific antibody with specificity for CD63 and GLA. In a particular embodiment, the enzyme is GLA or comprises GLA activity, the internalization domain is TfR, and the delivery domain is a bispecific antibody with specificity for TfR and GLA.

### Pharmaceutical Compositions and Administration Thereof

Pharmaceutical formulations may additionally comprise a pharmaceutically acceptable excipient, which, as used herein, includes any and all solvents, dispersion media, diluents, or other liquid vehicles, dispersion or suspension aids, surface active agents, isotonic agents, thickening or emulsifying agents, preservatives, solid binders, lubricants, and the like, as suited to the particular dosage form desired. Remington's The Science and Practice of Pharmacy, 21.sup.st Edition, A. R. Gennaro (Lippincott, Williams & Wilkins, Baltimore, Md., 2006; incorporated herein by reference in its entirety) discloses various excipients used in formulating pharmaceutical compositions and known techniques for the preparation thereof. Except insofar as any conventional excipient medium is incompatible with a substance or its derivatives, such as by producing any undesirable biological effect or otherwise interacting in a deleterious manner with any other component(s) of the pharmaceutical composition, its use is contemplated to be within the scope of this invention.

In some embodiments, a pharmaceutically acceptable excipient is at least 95%, at least 96%, at least 97%, at least 98%, at least 99%, or 100% pure. In some embodiments, an excipient is approved for use in humans and for veterinary use. In some embodiments, an excipient is approved by United States Food and Drug Administration. In some embodiments, an excipient is pharmaceutical grade. In some embodiments, an excipient meets the standards of the United States Pharmacopoeia (USP), the European Pharmacopoeia (EP), the British Pharmacopoeia, and/or the International Pharmacopoeia.

Pharmaceutically acceptable excipients used in the manufacture of pharmaceutical compositions include, but are not limited to, inert diluents, dispersing and/or granulating agents, surface active agents and/or emulsifiers, disintegrating agents, binding agents, preservatives, buffering agents, lubricating agents, and/or oils. Such excipients may optionally be included in pharmaceutical compositions.

Exemplary diluents include, but are not limited to, calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, dry starch, cornstarch, powdered sugar, etc., and/or combinations thereof.

Exemplary granulating and/or dispersing agents include, but are not limited to, potato starch, corn starch, tapioca starch, sodium starch glycolate, clays, alginic acid, guar gum, citrus pulp, agar, bentonite, cellulose and wood products, natural sponge, cation-exchange resins, calcium carbonate, silicates, sodium carbonate, cross-linked poly(vinyl-pyrrolidone) (crospovidone), sodium carboxymethyl starch (sodium starch glycolate), carboxymethyl cellulose, cross-linked sodium carboxymethyl cellulose (croscarmellose), methylcellulose, pregelatinized starch (starch 1500), microcrystalline starch, water insoluble starch, calcium carboxymethyl cellulose, magnesium aluminum silicate (VEEGUM®), sodium lauryl sulfate, quaternary ammonium compounds, etc., and/or combinations thereof.

Exemplary surface active agents and/or emulsifiers include, but are not limited to, natural emulsifiers (e.g. acacia, agar, alginic acid, sodium alginate, tragacanth, chondrux, cholesterol, xanthan, pectin, gelatin, egg yolk, casein, wool fat, cholesterol, wax, and lecithin), colloidal clays (e.g. bentonite [aluminum silicate] and VEEGUM® [magnesium aluminum silicate]), long chain amino acid derivatives, high molecular weight alcohols (e.g. stearyl alcohol, cetyl alcohol, oleyl alcohol, triacetin monostearate, ethylene glycol distearate, glyceryl monostearate, and propylene glycol monostearate, polyvinyl alcohol), carbomers (e.g. carboxy polymethylene, polyacrylic acid, acrylic acid polymer, and carboxyvinyl polymer), carrageenan, cellulosic derivatives (e.g. carboxymethylcellulose sodium, powdered cellulose, hydroxymethyl cellulose, hydroxypropyl cellulose, hydroxypropyl methylcellulose, methylcellulose), sorbitan fatty acid esters (e.g. polyoxyethylene sorbitan monolaurate [TWEEN® 20], polyoxyethylene sorbitan [TWEEN® 60], polyoxyethylene sorbitan monooleate [TWEEN® 80], sorbitan monopalmitate [SPAN® 40], sorbitan monostearate [SPAN® 60], sorbitan tristearate [SPAN® 65], glyceryl monooleate, sorbitan monooleate [SPAN® 80]), polyoxyethylene esters (e.g. polyoxyethylene monostearate [MYRJ® 45], polyoxyethylene hydrogenated castor oil, polyethoxylated castor oil, polyoxymethylene stearate, and SOLUTOL®), sucrose fatty acid esters, polyethylene glycol fatty acid esters (e.g. CREMOPHOR®), polyoxyethylene ethers, (e.g. polyoxyethylene lauryl ether [BRIJ® 30]), poly(vinyl-pyrrolidone), diethylene glycol monolaurate, triethanolamine oleate, sodium oleate, potassium oleate, ethyl oleate, oleic acid, ethyl laurate, sodium lauryl sulfate, PLUORINC® F 68, POLOXAMER® 188, cetrimonium bromide, cetylpyridinium chloride, benzalkonium chloride, docusate sodium, etc. and/or combinations thereof.

Exemplary binding agents include, but are not limited to, starch (e.g., cornstarch and starch paste); gelatin; sugars (e.g., sucrose, glucose, dextrose, dextrin, molasses, lactose, lactitol, mannitol); natural and synthetic gums (e.g., acacia, sodium alginate, extract of Irish moss, panwar gum, ghatti gum, mucilage of isapol husks, carboxymethylcellulose, methylcellulose, ethylcellulose, hydroxyethylcellulose, hydroxypropyl cellulose, hydroxypropyl methylcellulose, microcrystalline cellulose, cellulose acetate, poly(vinyl-pyrrolidone), magnesium aluminum silicate (Veegum®), and larch arabogalactan); alginates; polyethylene oxide; polyethylene glycol; inorganic calcium salts; silicic acid; polymethacrylates; waxes; water; alcohol; etc.; and combinations thereof.

Exemplary preservatives may include, but are not limited to, antioxidants, chelating agents, antimicrobial preservatives, antifungal preservatives, alcohol preservatives, acidic preservatives, and/or other preservatives. Exemplary antioxidants include, but are not limited to, alpha tocopherol, ascorbic acid, ascorbyl palmitate, butylated hydroxyanisole, butylated hydroxytoluene, monothioglycerol, potassium metabisulfite, propionic acid, propyl gallate, sodium ascorbate, sodium bisulfate, sodium metabisulfite, and/or sodium sulfite. Exemplary chelating agents include ethylenediaminetetraacetic acid (EDTA), citric acid monohydrate, disodium edetate, dipotassium edetate, edetic acid, fumaric acid, malic acid, phosphoric acid, sodium edetate, tartaric acid, and/or trisodium edetate. Exemplary antimicrobial preservatives include, but are not limited to, benzalkonium chloride, benzethonium chloride, benzyl alcohol, bronopol, cetrimide, cetylpyridinium chloride, chlorhexidine, chlorobutanol, chlorocresol, chloroxylenol, cresol, ethyl alcohol, glycerin, hexetidine, imidurea, phenol, phenoxyethanol, phenylethyl alcohol, phenylmercuric nitrate, propylene glycol, and/or thimerosal. Exemplary antifungal preservatives include, but are not limited to, butyl paraben, methyl paraben, ethyl paraben, propyl paraben, benzoic acid, hydroxybenzoic acid, potassium benzoate, potassium sorbate, sodium benzoate, sodium propionate, and/or sorbic acid. Exemplary alcohol preservatives include, but are not limited to, ethanol, polyethylene glycol, phenol, phenolic compounds, bisphenol, chlorobutanol, hydroxybenzoate, and/or phenylethyl alcohol. Exemplary acidic preservatives include, but are not limited to, vitamin A, vitamin C, vitamin E, beta-carotene, citric acid, acetic acid, dehydroacetic acid, ascorbic acid, sorbic acid, and/or phytic acid. Other preservatives include, but are not limited to, tocopherol, tocopherol acetate, deteroxime mesylate, cetrimide, butylated hydroxyanisol (BHA), butylated hydroxytoluene (BHT), ethylenediamine, sodium lauryl sulfate (SLS), sodium lauryl ether sulfate (SLES), sodium bisulfite, sodium metabisulfite, potassium sulfite, potassium metabisulfite, GLYDANT PLUS®, PHENONIP®, methylparaben, GERMALL® 115, GERMABEN® II, NEOLONE™, KATHON™, and/or EUXYL®.

Exemplary buffering agents include, but are not limited to, citrate buffer solutions, acetate buffer solutions, phosphate buffer solutions, ammonium chloride, calcium carbonate, calcium chloride, calcium citrate, calcium glubionate, calcium gluceptate, calcium gluconate, D-gluconic acid, calcium glycerophosphate, calcium lactate, propanoic acid, calcium levulinate, pentanoic acid, dibasic calcium phosphate, phosphoric acid, tribasic calcium phosphate, calcium hydroxide phosphate, potassium acetate, potassium chloride, potassium gluconate, potassium mixtures, dibasic potassium phosphate, monobasic potassium phosphate, potassium phosphate mixtures, sodium acetate, sodium bicarbonate, sodium chloride, sodium citrate, sodium lactate, dibasic sodium phosphate, monobasic sodium phosphate, sodium phosphate mixtures, tromethamine, magnesium hydroxide, aluminum hydroxide, alginic acid, pyrogen-free water, isotonic saline, Ringer's solution, ethyl alcohol, etc., and/or combinations thereof.

Exemplary lubricating agents include, but are not limited to, magnesium stearate, calcium stearate, stearic acid, silica, talc, malt, glyceryl behanate, hydrogenated vegetable oils, polyethylene glycol, sodium benzoate, sodium acetate, sodium chloride, leucine, magnesium lauryl sulfate, sodium lauryl sulfate, etc., and combinations thereof.

Exemplary oils include, but are not limited to, almond, apricot kernel, avocado, babassu, bergamot, black current seed, borage, cade, chamomile, canola, caraway, carnauba, castor, cinnamon, cocoa butter, coconut, cod liver, coffee, corn, cotton seed, emu, eucalyptus, evening primrose, fish, flaxseed, geraniol, gourd, grape seed, hazel nut, hyssop, isopropyl myristate, jojoba, kukui nut, lavandin, lavender, lemon, litsea cubeba, macadamia nut, mallow, mango seed, meadowfoam seed, mink, nutmeg, olive, orange, orange roughy, palm, palm kernel, peach kernel, peanut, poppy seed, pumpkin seed, rapeseed, rice bran, rosemary, safflower, sandalwood, sasquana, savoury, sea buckthorn, sesame, shea butter, silicone, soybean, sunflower, tea tree, thistle, tsubaki, vetiver, walnut, and wheat germ oils. Exemplary oils include, but are not limited to, butyl stearate, caprylic triglyceride, capric triglyceride, cyclomethicone, diethyl sebacate, dimethicone 360, isopropyl myristate, mineral oil, octyldodecanol, oleyl alcohol, silicone oil, and/or combinations thereof.

Excipients such as cocoa butter and suppository waxes, coloring agents, coating agents, sweetening, flavoring, and/or perfuming agents can be present in the composition, according to the judgment of the formulator.

### Delivery

The present disclosure encompasses the delivery of the gene therapy vector (e.g., the polynucleotides) by any appropriate route taking into consideration likely advances in the sciences of drug delivery. Delivery may be naked or formulated.

### Naked Delivery

The polynucleotides of the present invention may be delivered to a cell naked. As used herein in, “naked” refers to delivering polynucleotides free from agents that promote transfection. For example, the polynucleotides delivered to the cell may contain no modifications. The naked polynucleotides may be delivered to the cell using routes of administration known in the art and described herein.

### Formulated Delivery

The polynucleotides may be formulated, using the methods described herein. The formulations may contain polynucleotides and may further include, but are not limited to, cell penetration agents, a pharmaceutically acceptable carrier, a delivery agent, a bioerodible or biocompatible polymer, a solvent, and a sustained-release delivery depot. The formulated polynucleotides mRNA may be delivered to the cell using routes of administration known in the art and described herein.

### Administration

The polynucleotides of the present invention may be administered by any route which results in a therapeutically effective outcome. These include, but are not limited to enteral, gastroenteral, epidural, oral, transdermal, epidural (peridural), intracerebral (into the cerebrum), intracerebroventricular (into the cerebral ventricles), epicutaneous (application onto the skin), intradermal (into the skin itself), subcutaneous (under the skin), nasal administration (through the nose), intravenous (into a vein), intraarterial (into an artery), intramuscular (into a muscle), intracardiac (into the heart), intraosseous infusion (into the bone marrow), intrathecal (into the spinal canal), intraperitoneal (infusion or injection into the peritoneum), intravesical infusion, intravitreal (through the eye), intracavernous injection (into the base of the penis), intravaginal administration, intrauterine, extra-amniotic administration, transdermal (diffusion through the intact skin for systemic distribution), transmucosal (diffusion through a mucous membrane), insufflation (snorting), sublingual, sublabial, enema, eye drops (onto the conjunctiva), or in ear drops. In specific embodiments, compositions may be administered in a way that allows them to cross the blood-brain barrier, vascular barrier, or other epithelial barrier. Nonlimiting routes of administration for the polynucleotides, primary constructs, or mRNA of the present invention are described below.

### Parenteral and Injectable Administration

Liquid dosage forms for parenteral administration include, but are not limited to, pharmaceutically acceptable emulsions, microemulsions, solutions, suspensions, syrups, and/or elixirs. In addition to active ingredients, liquid dosage forms may comprise inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, but not limited to, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, oral compositions can include adjuvants such as wetting agents, emulsifying and suspending agents, sweetening, flavoring, and/or perfuming agents. In certain embodiments for parenteral administration, compositions are mixed with solubilizing agents such as CREMOPHOR®, alcohols, oils, modified oils, glycols, polysorbates, cyclodextrins, polymers, and/or combinations thereof.

Injectable preparations, for example, sterile injectable aqueous or oleaginous suspensions, may be formulated according to the known art using suitable dispersing agents, wetting agents, and/or suspending agents. Sterile injectable preparations may be sterile injectable solutions, suspensions, and/or emulsions in nontoxic parenterally acceptable diluents and/or solvents, for example, as a solution in 1,3-butanediol. Among the acceptable vehicles and solvents that may be employed are water, Ringer's solution, U.S.P., and isotonic sodium chloride solution. Sterile, fixed oils are conventionally employed as a solvent or suspending medium. For this purpose, any bland fixed oil can be employed including synthetic mono- or diglycerides. Fatty acids such as oleic acid can be used in the preparation of injectables.

Injectable formulations can be sterilized, for example, by filtration through a bacterial-retaining filter, and/or by incorporating sterilizing agents in the form of sterile solid compositions which can be dissolved or dispersed in sterile water or other sterile injectable medium prior to use.

In order to prolong the effect of an active ingredient, it is often desirable to slow the absorption of the active ingredient from subcutaneous or intramuscular injection. This may be accomplished by the use of a liquid suspension of crystalline or amorphous material with poor water solubility. The rate of absorption of the drug then depends upon its rate of dissolution which, in turn, may depend upon crystal size and crystalline form. Alternatively, delayed absorption of a parenterally administered drug form is accomplished by dissolving or suspending the drug in an oil vehicle. Injectable depot forms are made by forming microencapsulated matrices of the drug in biodegradable polymers such as polylactide-polyglycolide. Depending upon the ratio of drug to polymer and the nature of the particular polymer employed, the rate of drug release can be controlled. Examples of other biodegradable polymers include poly(orthoesters) and poly(anhydrides). Depot injectable formulations are prepared by entrapping the drug in liposomes or microemulsions which are compatible with body tissues.

### Depot Administration

As described herein, in some embodiments, the composition is formulated in depots for extended release. Generally, a specific organ or tissue (a “target tissue”) is targeted for administration.

In some aspects of the invention, the polynucleotides are spatially retained within or proximal to a target tissue. Provided are methods of providing a composition to a target tissue of a mammalian subject by contacting the target tissue (which contains one or more target cells) with the composition under conditions such that the composition, in particular the nucleic acid component(s) of the composition, is substantially retained in the target tissue, meaning that at least 10, 20, 30, 40, 50, 60, 70, 80, 85, 90, 95, 96, 97, 98, 99, 99.9, 99.99 or greater than 99.99% of the composition is retained in the target tissue. Advantageously, retention is determined by measuring the amount of the nucleic acid present in the composition that enters one or more target cells. For example, at least 1, 5, 10, 20, 30, 40, 50, 60, 70, 80, 85, 90, 95, 96, 97, 98, 99, 99.9, 99.99 or greater than 99.99% of the nucleic acids administered to the subject are present intracellularly at a period of time following administration. For example, intramuscular injection to a mammalian subject is performed using an aqueous composition containing a polynucleotide and a transfection reagent, and retention of the composition is determined by measuring the amount of the ribonucleic acid present in the muscle cells.

Aspects of the invention are directed to methods of providing a composition to a target tissue of a mammalian subject, by contacting the target tissue (containing one or more target cells) with the composition under conditions such that the composition is substantially retained in the target tissue. The composition contains an effective amount of a polynucleotide such that the polypeptide of interest is produced in at least one target cell. The compositions generally contain a cell penetration agent, although “naked” nucleic acid (such as nucleic acids without a cell penetration agent or other agent) is also contemplated, and a pharmaceutically acceptable carrier.

In some circumstances, the amount of a protein produced by cells in a tissue is desirably increased. Preferably, this increase in protein production is spatially restricted to cells within the target tissue. Thus, provided are methods of increasing production of a protein of interest in a tissue of a mammalian subject. A composition is provided that contains polynucleotides characterized in that a unit quantity of composition has been determined to produce the polypeptide of interest in a substantial percentage of cells contained within a predetermined volume of the target tissue.

In some embodiments, the composition includes a plurality of different polynucleotides, where one or more than one of the polynucleotides encodes a polypeptide of interest. Optionally, the composition also contains a cell penetration agent to assist in the intracellular delivery of the composition. A determination is made of the dose of the composition required to produce the polypeptide of interest in a substantial percentage of cells contained within the predetermined volume of the target tissue (generally, without inducing significant production of the polypeptide of interest in tissue adjacent to the predetermined volume, or distally to the target tissue). Subsequent to this determination, the determined dose is introduced directly into the tissue of the mammalian subject.

In one embodiment, the invention provides for the polynucleotides to be delivered in more than one injection or by split dose injections.

In one embodiment, the invention may be retained near target tissue using a small disposable drug reservoir, patch pump or osmotic pump. Nonlimiting examples of patch pumps include those manufactured and/or sold by BD® (Franklin Lakes, N.J.), Insulet Corporation (Bedford, Mass.), SteadyMed Therapeutics (San Francisco, Calif.), Medtronic (Minneapolis, Minn.) (e.g., MiniMed), UniLife (York, Pa.), Valeritas (Bridgewater, N.J.), and SpringLeaf Therapeutics (Boston, Mass.). Nonlimiting examples of osmotic pumps include those manufactured by DURECT® (Cupertino, Calif.) (e.g., DUROS® and ALZET®).

### Dosing

The present invention provides methods comprising administering a gene therapy vector comprising polynucleotide encoding a multidomain therapeutic polypeptide, and optionally subsequently the multidomain therapeutic polypeptide to a subject in need thereof. In some embodiments, a method comprises administering a gene therapy vector comprising a polynucleotide encoding a multidomain therapeutic polypeptide in a therapeutically effective amount to a patient in need thereof, wherein the therapeutically effective amount is sufficient to obviate the subsequent administration of the multidomain therapeutic polypeptide. Accordingly, in some embodiments, a method of treating a patient in need thereof lacking an enzyme, e.g., reducing glycogen levels and/or reducing CRIM to GAA in a patient with Pompe disease, comprises administering to the patient a gene therapy vector comprising a polynucleotide encoding a multidomain therapeutic protein comprising the replacement enzyme, e.g., an anti-TFRCscFv:GAA fusion protein, e.g., a multidomain therapeutic protein comprising the sequence set forth as SEQ ID NO:11, in a therapeutically effective amount, wherein the therapeutically effective amount negates the need for subsequent administration to the patient of the replacement enzyme, e.g., GAA or derivatives thereof. In some embodiments, a method of treating a patient lacking an enzyme and in need thereof, e.g., reducing glycogen levels and/or reducing CRIM to GAA in a patient with Pompe disease, comprises administering to the patient a gene therapy vector comprising a polynucleotide encoding a multidomain therapeutic protein comprising a replacement enzyme, e.g., an anti-TFRCscFv:GAA fusion protein, e.g., a multidomain therapeutic protein comprising the sequence set forth as SEQ ID NO:11, in a therapeutically effective amount, and further comprises administering to the patient a therapeutically effective amount of the replacement enzyme. Nucleic acids, proteins, or complexes, or pharmaceutical, imaging, diagnostic, or prophylactic compositions thereof, may be administered to a subject using any amount and any route of administration effective for preventing, treating, diagnosing, or imaging a disease, disorder, and/or condition (e.g., a disease, disorder, and/or condition relating to working memory deficits).

The exact amount required will vary from subject to subject, depending on the species, age, and general condition of the subject, the severity of the disease, the particular composition, its mode of administration, its mode of activity, and the like.

The dose of AAV viral vectors, e.g., the units of dose in vector genomes/per kilogram of body weight (vg/kg), required to achieve a desired effect or “therapeutic effect” (e.g., a certain serum concentration of a replacement enzyme) will vary based on several factors including, but not limited to: the route of AAV administration, the level of expression required to achieve a therapeutic effect, the specific disease or disorder being treated, and the stability of the expression multidomain therapeutic protein. One of skill in the art can readily determine a AAV virion dose range to treat a subject having a particular disease or disorder based on the aforementioned factors, as well as other factors that are well known in the art, see, e.g., CDER “Guidance for Industry Estimating the Maximum Safe Starting Dose in Initial Clinical Trials for Therapeutics in Adult Healthy Volunteers,” July 2005, incorporated herein in its entirety by reference. An effective amount of the AAV is generally in the range of from about 10 μl to about 100 ml of solution containing from about 10⁹ to 10¹⁶ genome copies per subject. Other volumes of solution may be used. The volume used will typically depend, among other things, on the size of the subject, the dose of the AAV, and the route of administration. In some embodiments, a dosage between about 10¹⁰ to 10¹² AAV viral genome per subject is appropriate. In some embodiments, the AAV is administered at a dose of 10¹⁰, 10¹¹, 10¹², 10¹3, 10¹⁴, or 10¹⁵ genome copies per subject. In some embodiments the AAV is administered at a dose of 10¹⁰, 10¹¹, 10¹², 10¹³, or 10¹⁴ viral genomes per kg. In some embodiments, at least 2×10¹² viral genomes per kilogram (vg/kg) is administered. In some embodiments, the dose administered provides a threshold multidomain therapeutic protein serum level. In some embodiments, the threshold therapeutic protein serum level is at least 0.5 μg/mL. In some embodiments, the threshold therapeutic protein serum level is at least 1 μg/mL. In some embodiments, the dose administered provides a multidomain therapeutic protein serum level of greater than 2 μg/mL. In some embodiments, the dose administered provides a multidomain therapeutic protein serum level of greater than 3 μg/mL. In some embodiments, the dose administered provides a multidomain therapeutic protein serum level of greater than 4 μg/mL. In some embodiments, the dose administered provides a multidomain therapeutic protein serum level of greater than 5 μg/mL. In some embodiments, the dose administered provides a multidomain therapeutic protein serum level of greater than 6 μg/mL. In some embodiments, the dose administered provides a multidomain therapeutic protein serum level of greater than 7 μg/mL. In some embodiments, the dose administered provides a multidomain therapeutic protein serum level of greater than 8 μg/mL. In some embodiments, the dose administered provides a multidomain therapeutic protein serum level of greater than 9 μg/mL. In some embodiments, the dose administered provides a multidomain therapeutic protein serum level of greater than 10 μg/mL. In some embodiments, the dose administered provides a multidomain therapeutic protein serum level of greater than 11 μg/mL. In some embodiments, the dose administered provides a multidomain therapeutic protein serum level of greater than 12 μg/mL. In some embodiments, the dose administered provides a multidomain therapeutic protein serum level of greater than 13 μg/mL. In some embodiments, the dose administered provides a multidomain therapeutic protein serum level of greater than 14 μg/mL. In some embodiments, the dose administered provides a multidomain therapeutic protein serum level of greater than 15 μg/mL.

Compositions in accordance with the invention are typically formulated in dosage unit form for ease of administration and uniformity of dosage. It will be understood, however, that the total daily usage of the compositions of the present invention may be decided by the attending physician within the scope of sound medical judgment. The specific therapeutically effective, prophylactically effective, or appropriate imaging dose level for any particular patient will depend upon a variety of factors including the disorder being treated and the severity of the disorder; the activity of the specific compound employed; the specific composition employed; the age, body weight, general health, sex, and diet of the patient; the time of administration, route of administration, and rate of excretion of the specific compound employed; the duration of the treatment; drugs used in combination or coincidental with the specific compound employed; and like factors well known in the medical arts.

The following examples are provided to further illustrate the methods of the present invention. These examples are illustrative only and are not intended to limit the scope of the invention in any way.

### Brief Description of the Sequences in the Sequence Listing

| SEQ ID NO: | Description |
|---|---|
| 1 | Human alpha glucosidase (GAA) protein |
| 2 | Anti-human CD63 scFv protein |
| 3 | Forward ITR primer |
| 4 | Reverse ITR primer |
| 5 | AAV2 ITR probe |
| 6 | 5′ AAV ITR |
| 7 | 3′ AAV ITR |
| 8 | TTR promoter |
| 9 | Serpinl |
| 10 | anti-hCD63scFv::hGAA fusion protein |
| 11 | an anti-TFRC scFv:GAA fusion protein |
| 12 | Human nucleic acid encoding alpha glucosidase (GAA) |
| 13 | Human alpha galactosidase (GAA) |
| 14 | AAV8 anti-(α)TFRCscfv:GAA plasmid sequence |
| 15 | HCVR amino acid sequence of 8D3 |
| 16 | HCDR1 amino acid sequence of 8D3 |
| 17 | HCDR2 amino acid sequence of 8D3 |
| 18 | HCDR3 amino acid sequence of 8D3 |
| 19 | LCVR amino acid sequence of 8D3 |
| 20 | LCDR1 amino acid sequence of 8D3 |
| 21 | LCDR2 amino acid sequence of 8D3 |
| 22 | LCDR3 amino acid sequence of 8D3 |
| 23 | 8D3 scFv amino acid sequence |

### EXAMPLES

### Example 1: αTFRCscfv:GAA Dose-Response Studies

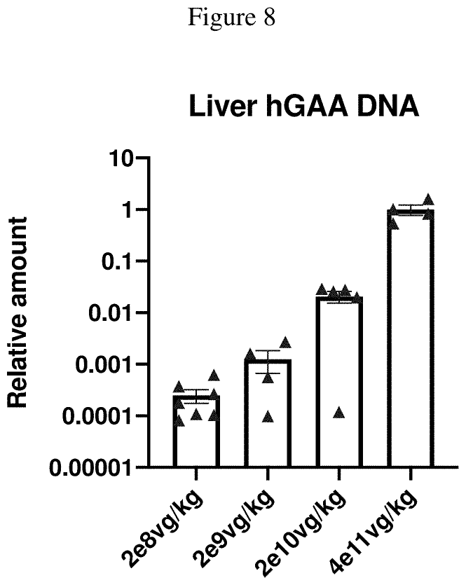

To determine the minimum required AAV dose in mice of αTFRCscfv:GAA, and to determine whether there is a dose-response relationship in glycogen clearance, the following experiments were conducted. Gaa−/− mice, humanized for the CD63 gene, were injected via tail vein with AAV8 expressing anti-TFRCscfv:GAA under the TTR promoter. The anti TfR antibody is anti-mouse comparator clone 8D3. GAA is human protein. Viral doses were determined by ddPCR; indicated doses range from 2.5e8 vg/kg to 4e11 vg/kg. Mice were treated at three months of age and harvested at four weeks post-injection. N=4-7 animals per group. Quantification of hGAA DNA and RNA expression in liver was measured by qPCR analysis. FIG. 8 shows the results for liver hGAA DNA, indicating relative amounts of hGAA DNA in relation to the amount with the highest viral dose.

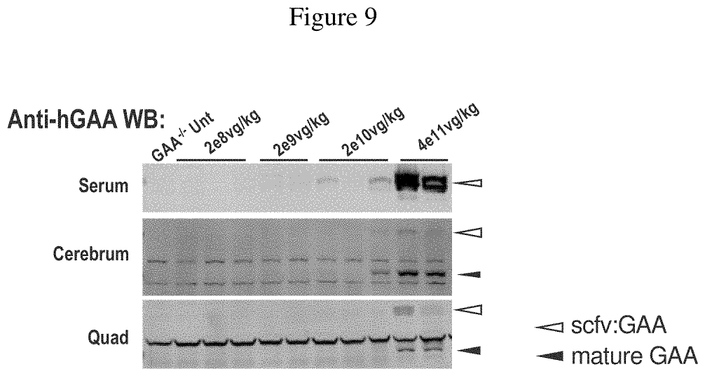

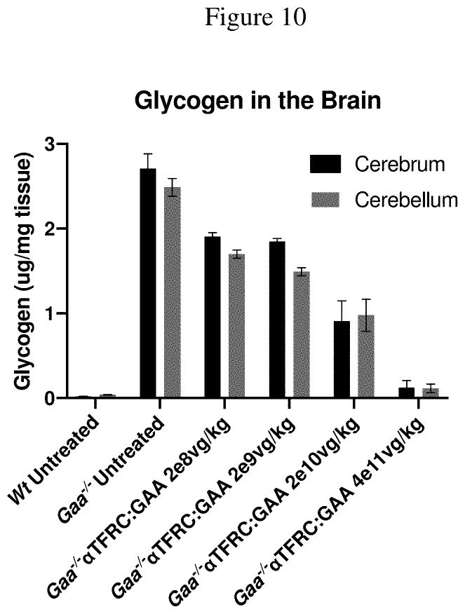

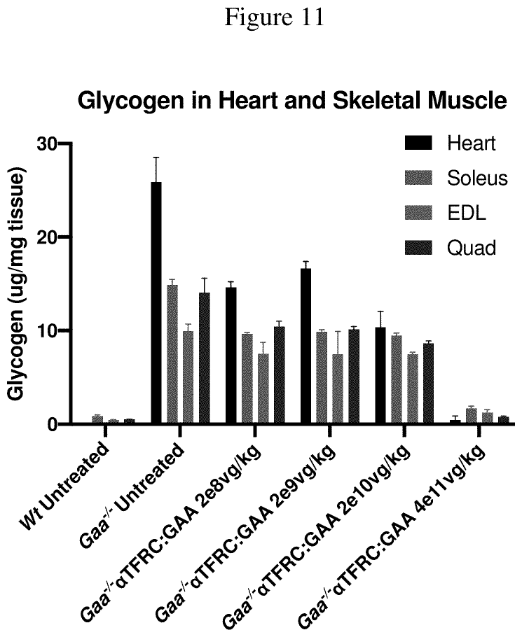

Further tissue analysis was done with an anti-hGAA western blot (FIG. 9). FIG. 9 shows the results for each of the doses tested in these experiments. Glycogen quantification in the brain (FIG. 10) and in muscle tissue (FIG. 11) was measured using Glycogen Assay Kit Sigma MAK016 (fluorometric). Both figures show that anti-TFRCscfv:GAA brings glycogen down to near-wildtype levels in cerebrum and cerebellum (FIG. 10) and in heart and skeletal muscle tissues (FIG. 11).

### Example 2: Effect of αTFRCscfv:GAA on Glycogen Levels in Brain

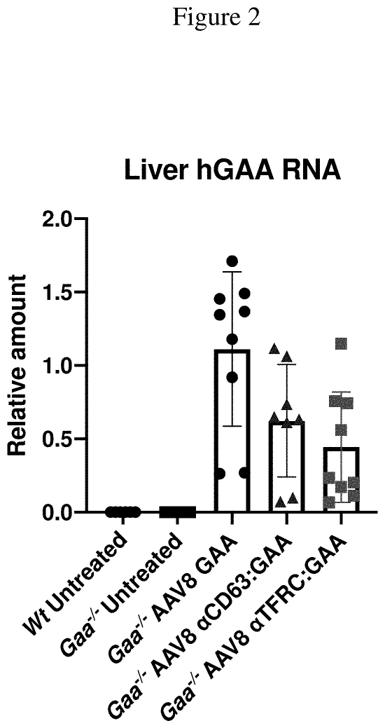

To compare glycogen levels in the brains of mice treated with AAV8 expressing αTfRscfv:GAA, αCD63scfv:GAA, and GAA, the following experiments were performed. Gaa−/− mice, humanized for the CD63 gene, were injected via tail vein with AAV8 expressing αTfRscfv:GAA, αCD63scfv:GAA, and GAA under the TTR promoter. The anti-TfR antibody is anti-mouse comparator clone 8D3. Anti CD63 is anti-human clone 12450. GAA is human protein. Viral doses were determined by ddPCR; the mice were dosed at 4e11 vg/kg. Mice were treated at three months of age and harvested at four weeks post-injection. N=6-10 animals per group. Quantification of hGAA DNA and RNA expression in liver was measured by qPCR analysis. FIG. 2 shows the results for liver hGAA RNA, indicating relative amounts of hGAA RNA in relation to the amount expressed with Gaa−/− AAV8 GAA.

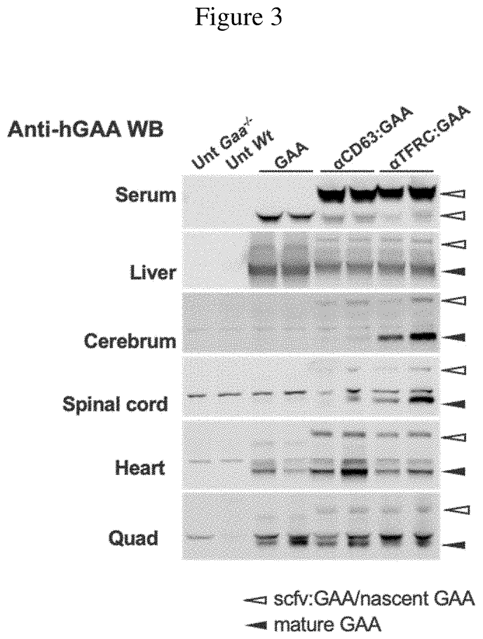

Tissue analysis was done with an anti-hGAA western blot (FIG. 3). FIG. 3 shows the results of several tissue types for Gaa−/− mice treated with AAV8 expressing either GAA, αCD63scfv:GAA, or αTFRCscfv:GAA under TTR promoter at dose of 4e11 vg/kg. The blot probed for hGAA. Each lane is an individual mouse.

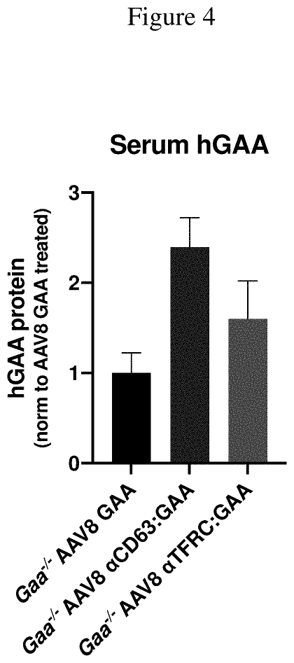

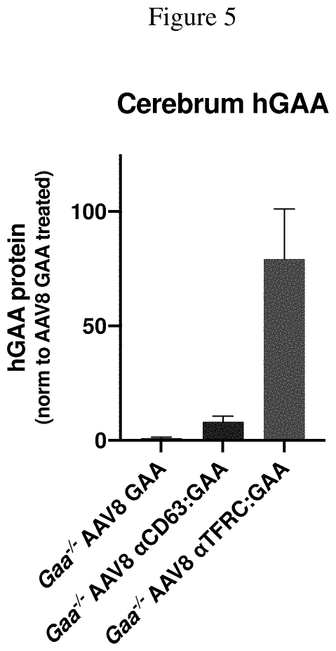

FIG. 4 shows quantification of GAA in serum from western blot in FIG. 3. FIG. 5 shows quantification of GAA in cerebrum from western blot in FIG. 3. Quantification is in arbitrary units, normalized to AAV8 GAA-treated.

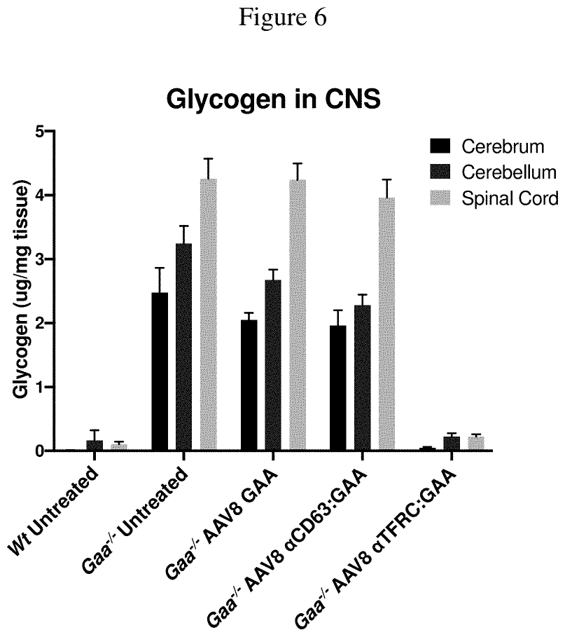

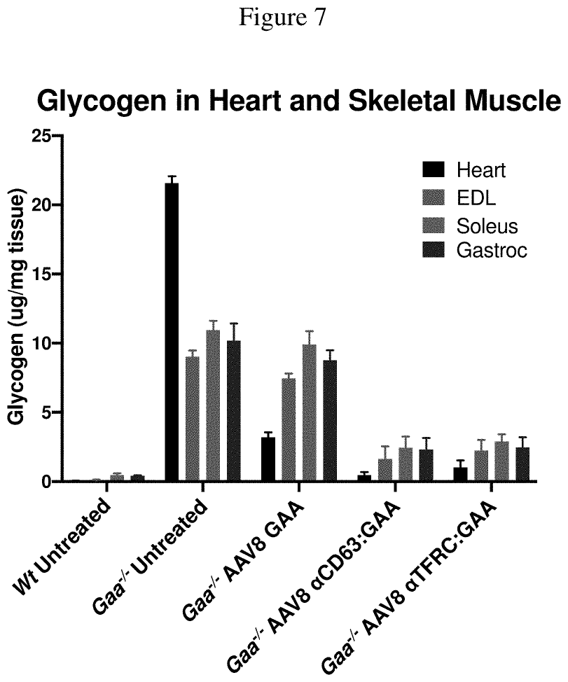

Glycogen quantification in the CNS (examining cerebrum, cerebellum, and spinal cord tissues; FIG. 6) and in heart and skeletal muscle tissues (FIG. 7) was measured using Glycogen Assay Kit Sigma MAK016 (fluorometric). FIG. 6 shows that αTFRCscfv:GAA alone brings glycogen levels down to near-wild type levels in the measured CNS tissues: cerebrum, cerebellum, and spinal cord. FIG. 7 shows that both αCD63scfv:GAA and αTFRCscfv:GAA bring glycogen levels down in heart and skeletal muscle tissues.

### Example 3: Immunofluorescence Imaging Studies

To demonstrate that αTFRCscfv:GAA is delivered to relevant cell types in the brain, as opposed to remaining trapped in BBB endothelial cells, the following experiments and data analyses were conducted. Gaa−/− mice, humanized for the CD63 gene, were injected via tail vein with AAV8 expressing anti-TFRCscfv:GAA under the TTR promoter. The anti TfR antibody is anti-mouse comparator clone 8D3. GAA is human protein. Viral dose was determined by ddPCR; the dose was 3.25e12 vg/kg. Mice were treated at three months of age and harvested at four weeks post-injection. N=3-4 animals per group.

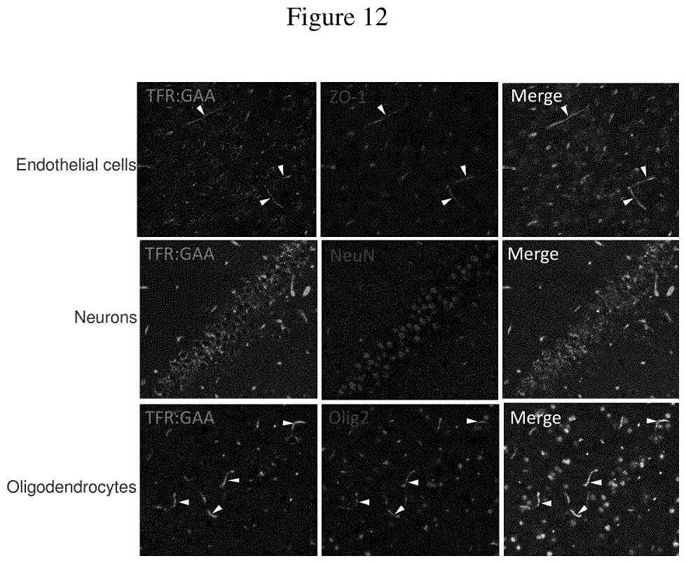

Harvesting and immunofluorescence staining proceeded as follows: mice were sacrificed, perfused, and coronal sections of cerebrum were prepared as formalin-fixed paraffin embedded (FFPE) on slides. Antigen retrieval was with basic HIER (heat-induced epitope retrieval). Sections were stained with anti-hGAA antibody, and costained for endothelial cell marker ZO-1, neuron marker NeuN, or oligodendrocyte marker Olig2. Antibodies used for analysis: Rabbit anti-GAA R&D systems MAB83291 (green); Mouse anti Zo-1 Millipore (red); Mouse anti NeuN Millipore MAB377 (red); Mouse anti Olig2 Millipore MABN50 (red); and DAPI nuclear marker (blue). FIG. 12 shows the immunofluorescence staining of brain sections from the experiments. These immunofluorescence findings demonstrate that αTFRCscfv:GAA is delivered to relevant cell types in the brain (i.e., neurons and oligodendrocytes), as opposed to remaining trapped in the endothelial cells of the BBB.

### Example 4: Quantification of GAA Activity

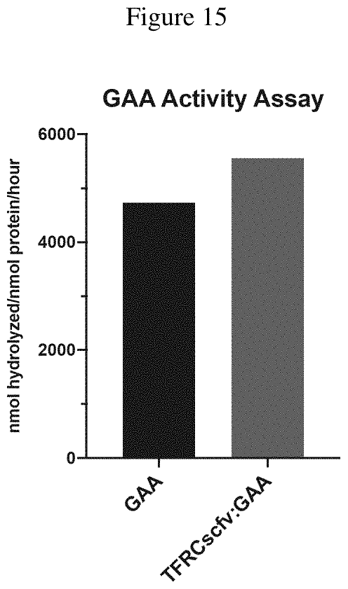

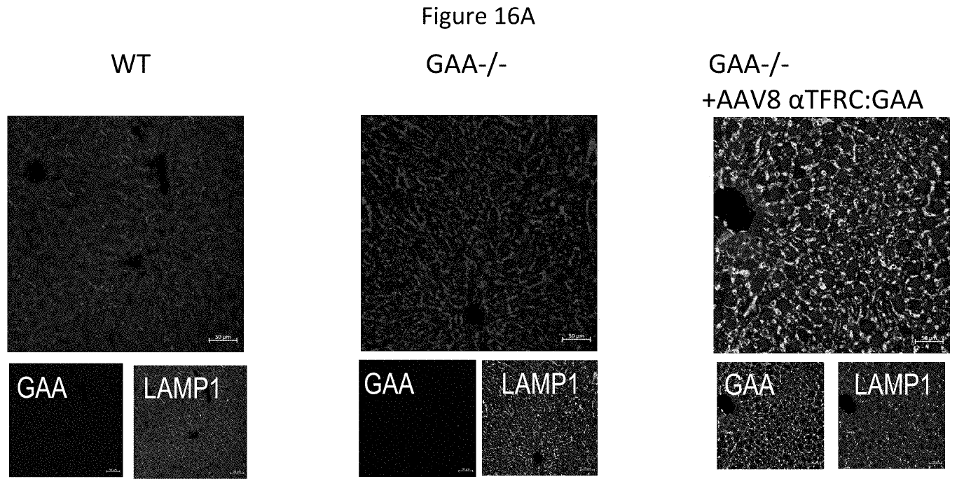

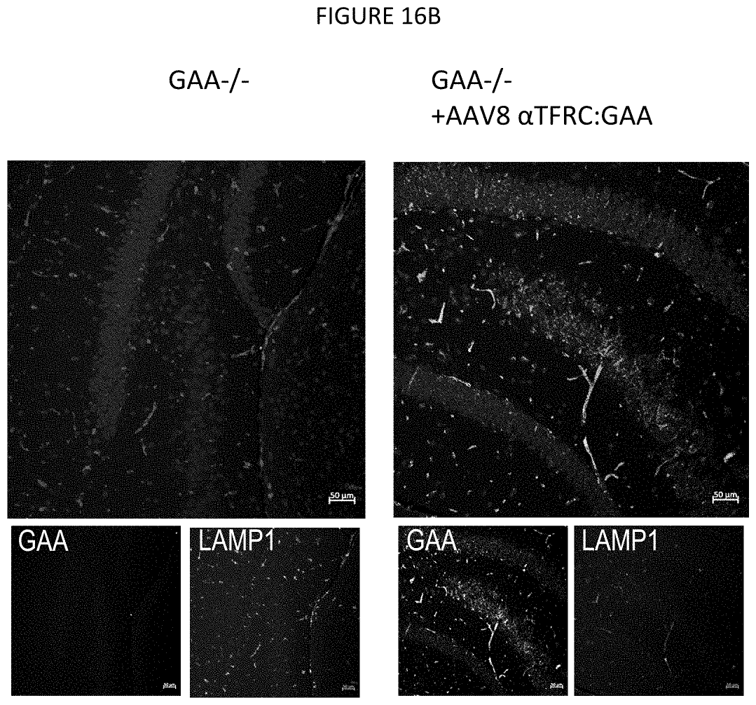

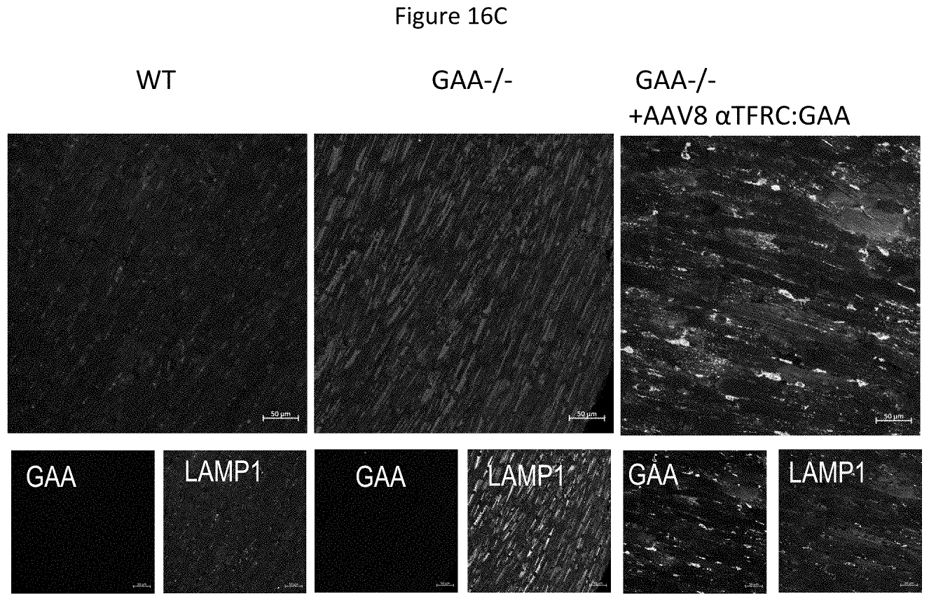

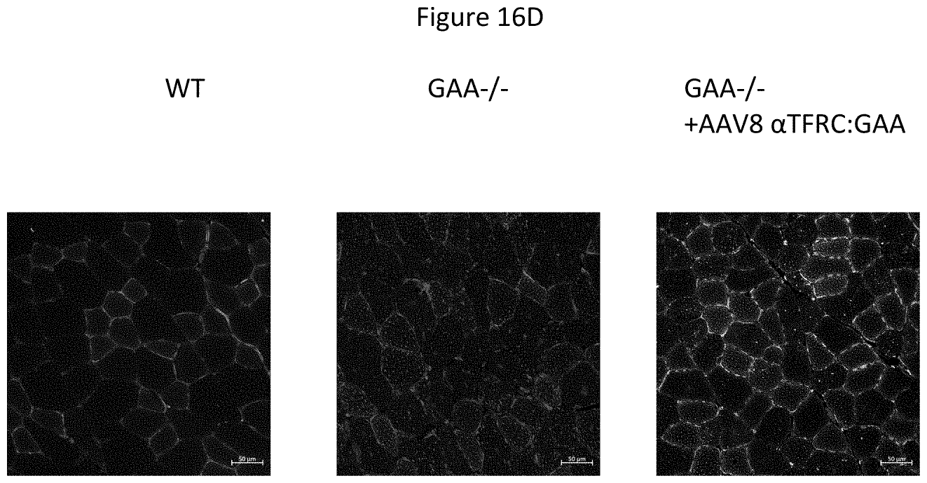

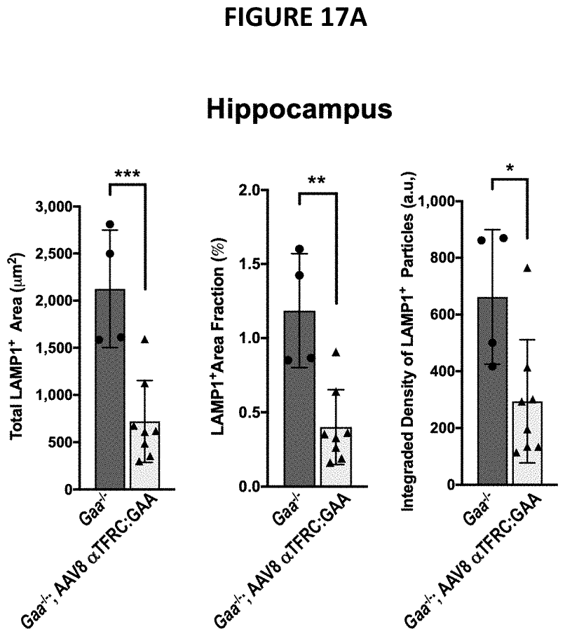

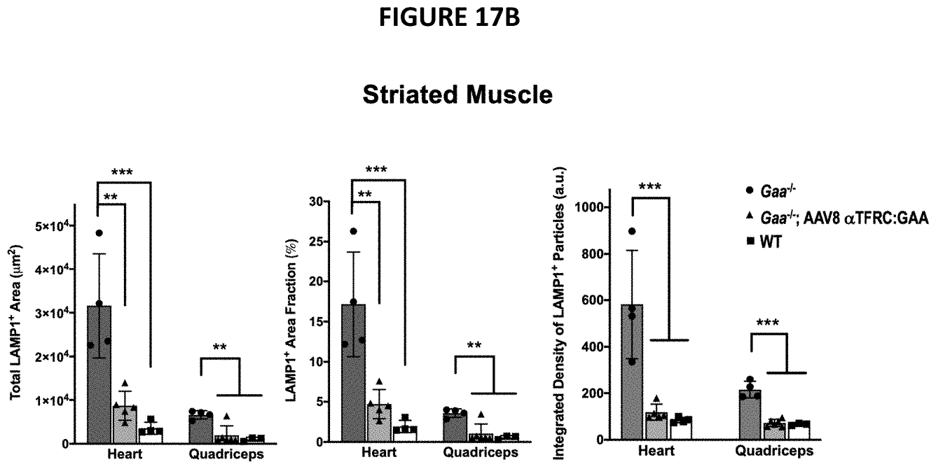

To determine quantification of GAA activity of purified hGAA protein (purchased from R&D Systems) and in-house purified αTFRCscfv:GAA, assays were performed as follows. Proteins were assayed for GAA activity with the fluorogenic substrate 4-methylumbelliferyl-alpha-D-glucopyranoside. 4-Methylumbelliferone was used as a standard. Purified protein GAA activity used a commercial fluorescence assay kit (K187, BioVision, Milpitas, Calif., USA). GAA activity was calculated as nanomoles of 4-methylumbelliferyl-alpha-D-glucopyranoside hydrolyzed per hour per nanomole of protein. As shown in FIG. 15, αTFRCscfv:GAA exhibited similar activity to the purified GAA protein.

### Example 5: Quantification of Lysosomal Area in the Brain by Imaging

To determine the percent lysosomal area, area fraction, and integrated density in hippocampus and striated muscles, mice were injected via tail vein with AAV8 expressing anti-TFRCscfv:GAA under the TTR promoter. After 4 weeks, mice were sacrificed, perfused, and coronal sections of cerebrum were prepared as formalin-fixed paraffin embedded samples on slides. Slides were de-paraffinized and then stained for antibodies to image lysosomes and GAA. For antibody staining, slides were blocked with tris buffer saline with 0.1% Tx-100 and 10% normal goat serum. They were subsequently stained with rat anti-Lamp1 1D4B (ab25245, Abcam, Cambridge, Mass., USA) and rabbit anti-GAA (MAB83291, R&D systems, Minneapolis, Minn., USA) to respectively label lysosomes and GAA distribution in hippocampus and striated muscles. Slides were subsequently stained with secondary antibodies goat anti-rat Alexa568 and anti-mouse Alexa488 (Thermo Fisher, Waltham, Mass., USA), mounted in Fluoromount-G with DAPI (Thermo Fisher, Waltham, Mass., USA), and imaged with a Zeiss LSM 710.

Images were quantified with ImageJ software. For measuring lysosomal area, area fraction and integrated density of Lamp1-positive particles, 3-8 images per group were analyzed. Total lysosomal area was determined as the percentage (%) of total Lamp1-positive area over the total area of the image. Integrated Density of Lamp1-positive particles is the product of mean density and total Lamp1-positive area. staining within the neuronal area. Results were compared with parallel experiments examining lysosomal area in wild-type mice, and GAA^{-/-} untreated mice. The studies show that lysosomal area, area fraction and integrated density were reduced in hippocampus and striated muscles following AAV8 anti-TfRC:GAA treatment. All parameters of Lamp1-positive particles in striated muscles of treated GAA−/− mice approached wild-type levels.

### Example 6: Quantification of Glycogen Storage in the Brain by Imaging

To determine the percent of glycogen storage in neuronal areas in the cerebrum, mice are injected via tail vein with AAV8 expressing anti-TFRCscfv:GAA under the TTR promoter. After 4 weeks, mice are sacrificed, perfused, and coronal sections of cerebrum are prepared as formalin-fixed paraffin embedded samples on slides. Slides are de-paraffinized and then stained for PAS-H (Epredia™ 87007 kit, Sigma Aldrich) to detect glycogen. Slides are then coverslipped and scanned on a Ventana slide scanner (Roche).

For PAS-H staining, neurons are identified by morphology and marked in the HALO software. Twenty neurons per region are outlined, and the PAS-stained area is quantified within this region. Results are compared with parallel experiments examining glycogen storage in wild-type, GAA^{-/-} untreated, and AAV8 anti-CD63:GAA treatment. The studies show that glycogen storage is reduced in neurons following AAV8 anti-TFRCscfv:GAA treatment, approaching wild-type levels.

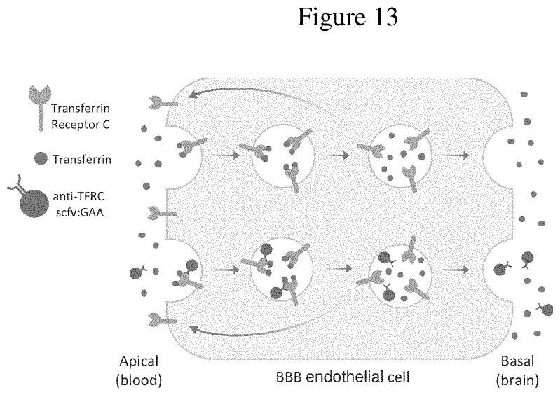

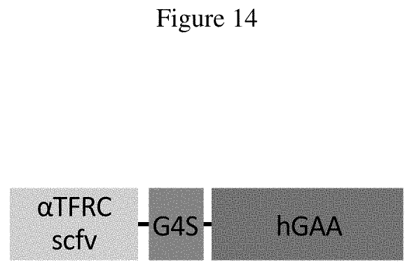

Without being bound by any one theory, the effects of AAV8 anti-TFRCscfv:GAA treatment in brain tissue, as shown, e.g., in FIG. 6, are the result of anti-TFRCscfv:GAA crossing the blood brain barrier through endothelial cells, as schematically depicted in FIG. 13. In addition, the therapeutic protein is reaching neurons and oligodendrocytes, as evidenced by, e.g., immunofluorescence studies (FIG. 12).

1. An antigen-binding protein that specifically binds to murine transferrin receptor, or an antigenic-fragment thereof or a variant thereof, comprising:
(i) an HCVR that comprises
an HCDR1 comprising the amino acid sequence set forth in SEQ ID NO:16,
an HCDR2 comprising the amino acid sequence set forth in SEQ ID NO:17, and/or
an HCDR3 comprising the amino acid sequence set forth in SEQ ID NO:18; and/or
(ii) an LCVR that comprises
an LCDR1 comprising the amino acid sequence set forth in SEQ ID NO:20,
an LCDR2 comprising the amino acid sequence set forth in SEQ ID NO:21, and
an LCDR3 comprising the amino acid sequence set forth in SEQ ID NO:22.

2. The antigen-binding protein of claim 1, wherein the HCVR comprises the amino acid sequence set forth in SEQ ID NO: 15 and the LCVR comprises the amino acid sequence set forth in SEQ ID NO:19.

3. The antigen-binding protein of claim 1, wherein the antigen-binding protein is an antibody or antigen-binding fragment thereof.

4. The antigen-binding protein of claim 3, wherein the antibody or antigen-binding is a Fab.

5. The antigen-binding protein of claim 3, wherein the antibody or antigen-binding is an scFv.

6. The antigen-binding protein of claim 5, wherein the scFv comprises the amino acid sequence set forth as SEQ ID NO:23.

7. A fusion protein comprising (i) the antigen-binding protein of claim 1 and (ii) a lysosomal enzyme.

8. The fusion protein of claim 7, wherein the lysosomal enzyme exhibits hydrolase activity.

9. The fusion protein of claim 7, wherein the lysosomal enzyme comprises alpha-glucosidase (GAA) or biologically active portion thereof, of alpha-galactosidase A or biologically active portion thereof.

10. The fusion protein of claim 7, wherein the lysosomal enzyme comprises GAA or a biologically active portion thereof.

11. The fusion of claim 7, wherein the fusion protein comprises the amino acid sequence set forth as SEQ ID NO:11.

12. A method of delivering a lysosomal enzyme to a central nervous system of a mouse comprising administering the fusion protein of claim 7 to the patient.

13. The method of claim 12, wherein administering comprises administering a nucleic acid comprising a sequence that encodes the fusion protein to the liver of the mouse.

14. The method of claim 13, wherein the nucleic acid is administered via a viral vector.

15. The method of claim 14, wherein the viral vector is an AAV vector, optionally wherein the AAV vector is administered at a dose of at least 2×10¹² viral genomes per kilogram (vg/kg).

16. The method of claim 13, wherein the nucleic acid further comprises a locus-targeting nucleic acid sequence and/or one or more tissue specific regulatory elements.

17. The method of claim 16, wherein the one or more tissue specific regulatory elements is a liver specific regulatory element, optionally wherein the liver specific regulatory element comprises a sequence set forth as SEQ ID NO:8 and/or SEQ ID NO:9.

18. The method of claim 16, wherein the one or more tissue specific regulatory elements is a neuronal specific promoter.

19. The method of claim 15, wherein the AAV vector comprises the nucleic acid sequence set forth as SEQ ID NO:14.

20. A gene therapy vector comprising the nucleic acid of claim 13.


SEQUENCE LISTING (23 sequences)

SEQ ID NO: 1 - protein, 952 aa, Homo sapiens
MGVRHPPCSHRLLAVCALVSLATAALLGHILLHDFLLVPRELSGSSPVLEETHPAHQQGASRPGPRDAQAHPGRPRAVPTQCDVPPNSRFDCAPDKAITQEQCEARGCCYIPAKQGLQGAQMGQPWCFFPPSYPSYKLENLSSSEMGYTATLTRTTPTFFPKDILTLRLDVMMETENRLHFTIKDPANRRYEVPLETPHVHSRAPSPLYSVEFSEEPFGVIVRRQLDGRVLLNTTVAPLFFADQFLQLSTSLPSQYITGLAEHLSPLMLSTSWTRITLWNRDLAPTPGANLYGSHPFYLALEDGGSAHGVFLLNSNAMDVVLQPSPALSWRSTGGILDVYIFLGPEPKSVVQQYLDVVGYPFMPPYWGLGFHLCRWGYSSTAITRQVVENMTRAHFPLDVQWNDLDYMDSRRDFTFNKDGFRDFPAMVQELHQGGRRYMMIVDPAISSSGPAGSYRPYDEGLRRGVFITNETGQPLIGKVWPGSTAFPDFTNPTALAWWEDMVAEFHDQVPFDGMWIDMNEPSNFIRGSEDGCPNNELENPPYVPGVVGGTLQAATICASSHQFLSTHYNLHNLYGLTEAIASHRALVKARGTRPFVISRSTFAGHGRYAGHWTGDVWSSWEQLASSVPEILQFNLLGVPLVGADVCGFLGNTSEELCVRWTQLGAFYPFMRNHNSLLSLPQEPYSFSEPAQQAMRKALTLRYALLPHLYTLFHQAHVAGETVARPLFLEFPKDSSTWTVDHQLLWGEALLITPVLQAGKAEVTGYFPLGTWYDLQTVPVEALGSLPPPPAAPREPAIHSEGQWVTLPAPLDTINVHLRAGYIIPLQGPGLTTTESRQQPMALAVALTKGGEARGELFWDDGESLEVLERGAYTQVIFLARNNTIVNELVRVTSEGAGLQLQKVTVLGVATAPQQVLSNGVPVSNFTYSPDTKVLDICVSLLMGEQFLVSWC

SEQ ID NO: 2 - protein, 245 aa, synthetic construct
  REGION 1..245: note = synthetic
EVKLVESGGGLVQPGGSLKLSCATSGFTFSDYYMSWVRQTPEKRLEWVAYISSSGGSTYYSDTVKGQFTISRDNAKNTLYLQMSRLKSEDTAMYYCARREDYDGRLTYWGQGTLVTISAGGSGGGGSGGGGSGGDIVLTQSPASLAVSLGQRATISCRASKSVSTSGYSYMNWYQQKPGQPPKVLIYLASKLESGVPARFSGSGSGTDFTLNIHPVEEEDAATYYCQHSRELPYTFGGGTKLEIK

SEQ ID NO: 3 - DNA, 21 bp, synthetic construct
  misc_feature 1..21: note = synthetic
  source 1..21: mol_type = other DNA
ggaacccctagtgatggagtt

SEQ ID NO: 4 - DNA, 16 bp, synthetic construct
  misc_feature 1..16: note = synthetic
  source 1..16: mol_type = other DNA
cggcctcagtgagcga

SEQ ID NO: 5 - DNA, 21 bp, synthetic construct
  misc_feature 1..21: note = synthetic
  source 1..21: mol_type = other DNA
cactccctctctgcgcgctcg

SEQ ID NO: 6 - DNA, 141 bp, Adeno-associated dependoparvovirus A
  misc_feature 1..141: note = synthetic
  source 1..141: mol_type = genomic DNA
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcct

SEQ ID NO: 7 - DNA, 141 bp, Adeno-associated dependoparvovirus A
  misc_feature 1..141: note = synthetic
  source 1..141: mol_type = other DNA
aggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 8 - DNA, 224 bp, synthetic construct
  misc_feature 1..224: note = synthetic
  source 1..224: mol_type = other DNA
gtctgtctgcacatttcgtagagcgagtgttccgatactctaatctccctaggcaaggttcatatttgtgtaggttacttattctccttttgttgactaagtcaataatcagaatcagcaggtttggagtcagcttggcagggatcagcagcctgggttggaaggagggggtataaaagccccttcaccaggagaagccgtcacacagatccacaagctcctga

SEQ ID NO: 9 - DNA, 72 bp, synthetic construct
  misc_feature 1..72: note = synthetic
  source 1..72: mol_type = other DNA
gggggaggctgctggtgaatattaaccaaggtcaccccagttatcggaggagcaaacaggggctaagtccac

SEQ ID NO: 10 - protein, 1133 aa, synthetic construct
  REGION 1..1133: note = synthetic
EVKLVESGGGLVQPGGSLKLSCATSGFTFSDYYMSWVRQTPEKRLEWVAYISSSGGSTYYSDTVKGQFTISRDNAKNTLYLQMSRLKSEDTAMYYCARREDYDGRLTYWGQGTLVTISAGGSGGGGSGGGGSGGDIVLTQSPASLAVSLGQRATISCRASKSVSTSGYSYMNWYQQKPGQPPKVLIYLASKLESGVPARFSGSGSGTDFTLNIHPVEEEDAATYYCQHSRELPYTFGGGTKLEIKGGGGSAHPGRPRAVPTQCDVPPNSRFDCAPDKAITQEQCEARGCCYIPAKQGLQGAQMGQPWCFFPPSYPSYKLENLSSSEMGYTATLTRTTPTFFPKDILTLRLDVMMETENRLHFTIKDPANRRYEVPLETPHVHSRAPSPLYSVEFSEEPFGVIVRRQLDGRVLLNTTVAPLFFADQFLQLSTSLPSQYITGLAEHLSPLMLSTSWTRITLWNRDLAPTPGANLYGSHPFYLALEDGGSAHGVFLLNSNAMDVVLQPSPALSWRSTGGILDVYIFLGPEPKSVVQQYLDVVGYPFMPPYWGLGFHLCRWGYSSTAITRQVVENMTRAHFPLDVQWNDLDYMDSRRDFTFNKDGFRDFPAMVQELHQGGRRYMMIVDPAISSSGPAGSYRPYDEGLRRGVFITNETGQPLIGKVWPGSTAFPDFTNPTALAWWEDMVAEFHDQVPFDGMWIDMNEPSNFIRGSEDGCPNNELENPPYVPGVVGGTLQAATICASSHQFLSTHYNLHNLYGLTEAIASHRALVKARGTRPFVISRSTFAGHGRYAGHWTGDVWSSWEQLASSVPEILQFNLLGVPLVGADVCGFLGNTSEELCVRWTQLGAFYPFMRNHNSLLSLPQEPYSFSEPAQQAMRKALTLRYALLPHLYTLFHQAHVAGETVARPLFLEFPKDSSTWTVDHQLLWGEALLITPVLQAGKAEVTGYFPLGTWYDLQTVPVEALGSLPPPPAAPREPAIHSEGQWVTLPAPLDTINVHLRAGYIIPLQGPGLTTTESRQQPMALAVALTKGGEARGELFWDDGESLEVLERGAYTQVIFLARNNTIVNELVRVTSEGAGLQLQKVTVLGVATAPQQVLSNGVPVSNFTYSPDTKVLDICVSLLMGEQFLVSWC

SEQ ID NO: 11 - DNA, 3489 bp, synthetic construct
  misc_feature 1..3489: note = synthetic
  source 1..3489: mol_type = other DNA
atgcacagacctagacgtcgtggaactcgtccacctccactggcactgctcgctgctctcctcctggctgcacgtggtgctgatgcagaagtgaagctggtggagtctgggggaggcttagtgcagcctggagggtccctgaaactctcctgtgcaacctctggattcactttcagtgactattacatgtcttgggttcgccagactccagagaagaggctggagtgggtcgcatatattagtagtagtggtggtagcacctattattcagacactgtaaagggccaattcaccatctccagagacaatgccaagaacaccctgtacctgcaaatgagccgtctgaagtctgaggacacagccatgtattactgtgcaagacgagaagattacgacggaagacttacttactggggccaagggactctggtcaccatctctgcaggaggaagtggtggaggcgggtcaggaggtggcgggagcggcggtgacattgtgctgacacagtctcctgcttccttagctgtatctctggggcagagggccaccatctcctgcagggccagcaaaagtgtcagtacatctggttatagttatatgaactggtaccaacagaaaccaggacagccacccaaagtcctcatctatcttgcatccaaactagaatctggggtccctgccaggttcagtggcagtgggtcagggacagacttcaccctcaacatccatcctgtggaggaggaggatgctgcaacctattactgtcagcacagtagggagcttccgtacacgttcggaggggggaccaaactggaaataaaaggtggtggcggttcagcacaccccggccgtcccagagcagtgcccacacagtgcgacgtcccccccaacagccgcttcgattgcgcccctgacaaggccatcacccaggaacagtgcgaggcccgcggctgttgctacatccctgcaaagcaggggctgcagggagcccagatggggcagccctggtgcttcttcccacccagctaccccagctacaagctggagaacctgagctcctctgaaatgggctacacggccaccctgacccgtaccacccccaccttcttccccaaggacatcctgaccctgcggctggacgtgatgatggagactgagaaccgcctccacttcacgatcaaagatccagctaacaggcgctacgaggtgcccttggagaccccgcatgtccacagccgggcaccgtccccactctacagcgtggagttctccgaggagcccttcggggtgatcgtgcgccggcagctggacggccgcgtgctgctgaacacgacggtggcgcccctgttctttgcggaccagttccttcagctgtccacctcgctgccctcgcagtatatcacaggcctcgccgagcacctcagtcccctgatgctcagcaccagctggaccaggatcaccctgtggaaccgggaccttgcgcccacgcccggtgcgaacctctacgggtctcaccctttctacctggcgctggaggacggcgggtcggcacacggggtgttcctgctaaacagcaatgccatggatgtggtcctgcagccgagccctgcccttagctggaggtcgacaggtgggatcctggatgtctacatcttcctgggcccagagcccaagagcgtggtgcagcagtacctggacgttgtgggatacccgttcatgccgccatactggggcctgggcttccacctgtgccgctggggctactcctccaccgctatcacccgccaggtggtggagaacatgaccagggcccacttccccctggacgtccagtggaacgacctggactacatggactcccggagggacttcacgttcaacaaggatggcttccgggacttcccggccatggtgcaggagctgcaccagggcggccggcgctacatgatgatcgtggatcctgccatcagcagctcgggccctgccgggagctacaggccctacgacgagggtctgcggaggggggttttcatcaccaacgagaccggccagccgctgattgggaaggtatggcccgggtccactgccttccccgacttcaccaaccccacagccctggcctggtgggaggacatggtggctgagttccatgaccaggtgcccttcgacggcatgtggattgacatgaacgagccttccaacttcatcaggggctctgaggacggctgccccaacaatgagctggagaacccaccctacgtgcctggggtggttggggggaccctccaggcggccaccatctgtgcctccagccaccagtttctctccacacactacaacctgcacaacctctacggcctgaccgaagccatcgcctcccacagggcgctggtgaaggctcgggggacacgcccatttgtgatctcccgctcgacctttgctggccacggccgatacgccggccactggacgggggacgtgtggagctcctgggagcagctcgcctcctccgtgccagaaatcctgcagtttaacctgctgggggtgcctctggtcggggccgacgtctgcggcttcctgggcaacacctcagaggagctgtgtgtgcgctggacccagctgggggccttctaccccttcatgcggaaccacaacagcctgctcagtctgccccaggagccgtacagcttcagcgagccggcccagcaggccatgaggaaggccctcaccctgcgctacgcactcctcccccacctctacacactgttccaccaggcccacgtcgcgggggagaccgtggcccggcccctcttcctggagttccccaaggactctagcacctggactgtggaccaccagctcctgtggggggaggccctgctcatcaccccagtgctccaggccgggaaggccgaagtgactggctacttccccttgggcacatggtacgacctgcagacggtgccagtagaggcccttggcagcctcccacccccacctgcagctccccgtgagccagccatccacagcgaggggcagtgggtgacgctgccggcccccctggacaccatcaacgtccacctccgggctgggtacatcatccccctgcagggccctggcctcacaaccacagagtcccgccagcagcccatggccctggctgtggccctgaccaagggtggggaggcccgaggggagctgttctgggacgatggagagagcctggaagtgctggagcgaggggcctacacacaggtcatcttcctggccaggaataacacgatcgtgaatgagctggtacgtgtgaccagtgagggagctggcctgcagctgcagaaggtgactgtcctgggcgtggccacggcgccccagcaggtcctctccaacggtgtccctgtctccaacttcacctacagccccgacaccaaggtcctggacatctgtgtctcgctgttgatgggagagcagtttctcgtcagctggtgttag

SEQ ID NO: 12 - DNA, 2859 bp, Homo sapiens
  source 1..2859: mol_type = genomic DNA
atgggagtgaggcacccgccctgctcccaccggctcctggccgtctgcgccctcgtgtccttggcaaccgctgcactcctggggcacatcctactccatgatttcctgctggttccccgagagctgagtggctcctccccagtcctggaggagactcacccagctcaccagcagggagccagcagaccagggccccgggatgcccaggcacaccccggccgtcccagagcagtgcccacacagtgcgacgtcccccccaacagccgcttcgattgcgcccctgacaaggccatcacccaggaacagtgcgaggcccgcggctgttgctacatccctgcaaagcaggggctgcagggagcccagatggggcagccctggtgcttcttcccacccagctaccccagctacaagctggagaacctgagctcctctgaaatgggctacacggccaccctgacccgtaccacccccaccttcttccccaaggacatcctgaccctgcggctggacgtgatgatggagactgagaaccgcctccacttcacgatcaaagatccagctaacaggcgctacgaggtgcccttggagaccccgcatgtccacagccgggcaccgtccccactctacagcgtggagttctccgaggagcccttcggggtgatcgtgcgccggcagctggacggccgcgtgctgctgaacacgacggtggcgcccctgttctttgcggaccagttccttcagctgtccacctcgctgccctcgcagtatatcacaggcctcgccgagcacctcagtcccctgatgctcagcaccagctggaccaggatcaccctgtggaaccgggaccttgcgcccacgcccggtgcgaacctctacgggtctcaccctttctacctggcgctggaggacggcgggtcggcacacggggtgttcctgctaaacagcaatgccatggatgtggtcctgcagccgagccctgcccttagctggaggtcgacaggtgggatcctggatgtctacatcttcctgggcccagagcccaagagcgtggtgcagcagtacctggacgttgtgggatacccgttcatgccgccatactggggcctgggcttccacctgtgccgctggggctactcctccaccgctatcacccgccaggtggtggagaacatgaccagggcccacttccccctggacgtccagtggaacgacctggactacatggactcccggagggacttcacgttcaacaaggatggcttccgggacttcccggccatggtgcaggagctgcaccagggcggccggcgctacatgatgatcgtggatcctgccatcagcagctcgggccctgccgggagctacaggccctacgacgagggtctgcggaggggggttttcatcaccaacgagaccggccagccgctgattgggaaggtatggcccgggtccactgccttccccgacttcaccaaccccacagccctggcctggtgggaggacatggtggctgagttccatgaccaggtgcccttcgacggcatgtggattgacatgaacgagccttccaacttcatcaggggctctgaggacggctgccccaacaatgagctggagaacccaccctacgtgcctggggtggttggggggaccctccaggcggccaccatctgtgcctccagccaccagtttctctccacacactacaacctgcacaacctctacggcctgaccgaagccatcgcctcccacagggcgctggtgaaggctcgggggacacgcccatttgtgatctcccgctcgacctttgctggccacggccgatacgccggccactggacgggggacgtgtggagctcctgggagcagctcgcctcctccgtgccagaaatcctgcagtttaacctgctgggggtgcctctggtcggggccgacgtctgcggcttcctgggcaacacctcagaggagctgtgtgtgcgctggacccagctgggggccttctaccccttcatgcggaaccacaacagcctgctcagtctgccccaggagccgtacagcttcagcgagccggcccagcaggccatgaggaaggccctcaccctgcgctacgcactcctcccccacctctacacactgttccaccaggcccacgtcgcgggggagaccgtggcccggcccctcttcctggagttccccaaggactctagcacctggactgtggaccaccagctcctgtggggggaggccctgctcatcaccccagtgctccaggccgggaaggccgaagtgactggctacttccccttgggcacatggtacgacctgcagacggtgccagtagaggcccttggcagcctcccacccccacctgcagctccccgtgagccagccatccacagcgaggggcagtgggtgacgctgccggcccccctggacaccatcaacgtccacctccgggctgggtacatcatccccctgcagggccctggcctcacaaccacagagtcccgccagcagcccatggccctggctgtggccctgaccaagggtggggaggcccgaggggagctgttctgggacgatggagagagcctggaagtgctggagcgaggggcctacacacaggtcatcttcctggccaggaataacacgatcgtgaatgagctggtacgtgtgaccagtgagggagctggcctgcagctgcagaaggtgactgtcctgggcgtggccacggcgccccagcaggtcctctccaacggtgtccctgtctccaacttcacctacagccccgacaccaaggtcctggacatctgtgtctcgctgttgatgggagagcagtttctcgtcagctggtgttag

SEQ ID NO: 13 - protein, 398 aa, Homo sapiens
LDNGLARTPTMGWLHWERFMCNLDCQEEPDSCISEKLFMEMAELMVSEGWKDAGYEYLCIDDCWMAPQRDSEGRLQADPQRFPHGIRQLANYVHSKGLKLGIYADVGNKTCAGFPGSFGYYDIDAQTFADWGVDLLKFDGCYCDSLENLADGYKHMSLALNRTGRSIVYSCEWPLYMWPFQKPNYTEIRQYCNHWRNFADIDDSWKSIKSILDWTSFNQERIVDVAGPGGWNDPDMLVIGNFGLSWNQQVTQMALWAIMAAPLFMSNDLRHISPQAKALLQDKDVIAINQDPLGKQGYQLRQGDNFEVWERPLSGLAWAVAMINRQEIGGPRSYTIAVASLGKGVACNPACFITQLLPVKRKLGFYEWTSRLRSHINPTGTVLLQLENTMQMSLKDLL

SEQ ID NO: 14 - DNA, 7015 bp, synthetic construct
  misc_feature 1..7015
  source 1..7015: mol_type = other DNA
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcacgcgtggtaccgggggaggctgctggtgaatattaaccaaggtcaccccagttatcggaggagcaaacaggggctaagtccacagccgtggtaccgtctgtctgcacatttcgtagagcgagtgttccgatactctaatctccctaggcaaggttcatatttgtgtaggttacttattctccttttgttgactaagtcaataatcagaatcagcaggtttggagtcagcttggcagggatcagcagcctgggttggaaggagggggtataaaagccccttcaccaggagaagccgtcacacagatccacaagctcctgaaagcttatactcgagctctagattgggaacccgggtctctcgaattcgagatctccaccatgcacagacctagacgtcgtggaactcgtccacctccactggcactgctcgctgctctcctcctggctgcacgtggtgctgatgcagaagtacagctcgtcgaaagtggcggtggtcttgttcagcctggaaactccctcaccctctcatgtgtcgcaagtggatttactttctcaaattatggaatgcattggattaggcaagcaccaaaaaaaggacttgaatggatagctatgatatattacgactcctctaaaatgaattacgcagatactgtaaaaggcaggtttacaatttctcgggataactctaaaaacactctttaccttgaaatgaactctcttagatccgaagatacagctatgtactattgcgcagtgccaactagtcattacgtcgtcgatgtttggggacaaggcgtaagtgtaacagtcagctcaggcggcggcggaagcggagggggaggatcaggaggaggcggatccgacattcagatgacacaatccccagcatccctctcagcatcactcgaagaaatcgtaacaatcacctgtcaagcatctcaagatatcggcaactggctcgcatggtaccaacagaaacccggcaaatcccctcaacttcttatttatggcgcaacatccctcgcagacggcgttccctcaaggttctcaggctcacgatcaggcactcagttctcacttaaaatctccagagttcaagtcgaagatataggaatttattattgtctccaagcatataacacaccatggacattcggcggcggcaccaaactcgaacttaaaggaggtggtggaagtggaggtggtggaagtgcacaccccggccgtcccagagcagtgcccacacagtgcgacgtcccccccaacagccgcttcgattgcgcccctgacaaggccatcacccaggaacagtgcgaggcccgcggctgttgctacatccctgcaaagcaggggctgcagggagcccagatggggcagccctggtgcttcttcccacccagctaccccagctacaagctggagaacctgagctcctctgaaatgggctacacggccaccctgacccgtaccacccccaccttcttccccaaggacatcctgaccctgcggctggacgtgatgatggagactgagaaccgcctccacttcacgatcaaagatccagctaacaggcgctacgaggtgcccttggagaccccgcatgtccacagccgggcaccgtccccactctacagcgtggagttctccgaggagcccttcggggtgatcgtgcgccggcagctggacggccgcgtgctgctgaacacgacggtggcgcccctgttctttgcggaccagttccttcagctgtccacctcgctgccctcgcagtatatcacaggcctcgccgagcacctcagtcccctgatgctcagcaccagctggaccaggatcaccctgtggaaccgggaccttgcgcccacgcccggtgcgaacctctacgggtctcaccctttctacctggcgctggaggacggcgggtcggcacacggggtgttcctgctaaacagcaatgccatggatgtggtcctgcagccgagccctgcccttagctggaggtcgacaggtgggatcctggatgtctacatcttcctgggcccagagcccaagagcgtggtgcagcagtacctggacgttgtgggatacccgttcatgccgccatactggggcctgggcttccacctgtgccgctggggctactcctccaccgctatcacccgccaggtggtggagaacatgaccagggcccacttccccctggacgtccagtggaacgacctggactacatggactcccggagggacttcacgttcaacaaggatggcttccgggacttcccggccatggtgcaggagctgcaccagggcggccggcgctacatgatgatcgtggatcctgccatcagcagctcgggccctgccgggagctacaggccctacgacgagggtctgcggaggggggttttcatcaccaacgagaccggccagccgctgattgggaaggtatggcccgggtccactgccttccccgacttcaccaaccccacagccctggcctggtgggaggacatggtggctgagttccatgaccaggtgcccttcgacggcatgtggattgacatgaacgagccttccaacttcatcaggggctctgaggacggctgccccaacaatgagctggagaacccaccctacgtgcctggggtggttggggggaccctccaggcggccaccatctgtgcctccagccaccagtttctctccacacactacaacctgcacaacctctacggcctgaccgaagccatcgcctcccacagggcgctggtgaaggctcgggggacacgcccatttgtgatctcccgctcgacctttgctggccacggccgatacgccggccactggacgggggacgtgtggagctcctgggagcagctcgcctcctccgtgccagaaatcctgcagtttaacctgctgggggtgcctctggtcggggccgacgtctgcggcttcctgggcaacacctcagaggagctgtgtgtgcgctggacccagctgggggccttctaccccttcatgcggaaccacaacagcctgctcagtctgccccaggagccgtacagcttcagcgagccggcccagcaggccatgaggaaggccctcaccctgcgctacgcactcctcccccacctctacacactgttccaccaggcccacgtcgcgggggagaccgtggcccggcccctcttcctggagttccccaaggactctagcacctggactgtggaccaccagctcctgtggggggaggccctgctcatcaccccagtgctccaggccgggaaggccgaagtgactggctacttccccttgggcacatggtacgacctgcagacggtgccagtagaggcccttggcagcctcccacccccacctgcagctccccgtgagccagccatccacagcgaggggcagtgggtgacgctgccggcccccctggacaccatcaacgtccacctccgggctgggtacatcatccccctgcagggccctggcctcacaaccacagagtcccgccagcagcccatggccctggctgtggccctgaccaagggtggggaggcccgaggggagctgttctgggacgatggagagagcctggaagtgctggagcgaggggcctacacacaggtcatcttcctggccaggaataacacgatcgtgaatgagctggtacgtgtgaccagtgagggagctggcctgcagctgcagaaggtgactgtcctgggcgtggccacggcgccccagcaggtcctctccaacggtgtccctgtctccaacttcacctacagccccgacaccaaggtcctggacatctgtgtctcgctgttgatgggagagcagtttctcgtcagctggtgttaggcggccgctaatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtcgatgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 15 - protein, 118 aa, synthetic construct
EVQLVESGGGLVQPGNSLTLSCVASGFTFSNYGMHWIRQAPKKGLEWIAMIYYDSSKMNYADTVKGRFTISRDNSKNTLYLEMNSLRSEDTAMYYCAVPTSHYVVDVWGQGVSVTVSS

SEQ ID NO: 16 - protein, 10 aa, synthetic construct
GFTFSNYGMH

SEQ ID NO: 17 - protein, 17 aa, synthetic construct
MIYYDSSKMNYADTVKG

SEQ ID NO: 18 - protein, 9 aa, synthetic construct
PTSHYVVDV

SEQ ID NO: 19 - protein, 107 aa, synthetic construct
DIQMTQSPASLSASLEEIVTITCQASQDIGNWLAWYQQKPGKSPQLLIYGATSLADGVPSRFSGSRSGTQFSLKISRVQVEDIGIYYCLQAYNTPWTFGGGTKLELK

SEQ ID NO: 20 - protein, 11 aa, synthetic construct
QASQDIGNWLA

SEQ ID NO: 21 - protein, 7 aa, synthetic construct
GATSLAD

SEQ ID NO: 22 - protein, 9 aa, synthetic construct
LQAYNTPWT

SEQ ID NO: 23 - protein, 240 aa, synthetic construct
EVQLVESGGGLVQPGNSLTLSCVASGFTFSNYGMHWIRQAPKKGLEWIAMIYYDSSKMNYADTVKGRFTISRDNSKNTLYLEMNSLRSEDTAMYYCAVPTSHYVVDVWGQGVSVTVSSGGGGSGGGGSGGGGSDIQMTQSPASLSASLEEIVTITCQASQDIGNWLAWYQQKPGKSPQLLIYGATSLADGVPSRFSGSRSGTQFSLKISRVQVEDIGIYYCLQAYNTPWTFGGGTKLELK